(19) United States
(12) Patent Application Publication
(10) Pub. No.: US 2024/0116875 A1
(43) Pub. Date: Apr. 11, 2024

(54) RATIONAL DESIGN, OPTIMIZATION, AND BIOLOGICAL EVALUATION OF NOVEL MEK4 INHIBITORS AGAINST PANCREATIC ADENOCARCINOMA

(71) Applicant: NORTHWESTERN UNIVERSITY, Evanston, IL (US)

(72) Inventors: Karl A. Scheidt, Evanston, IL (US); Hidayatullah G. Munshi, Evanston, IL (US); Ada Jade Kwong, Evanston, IL (US); Thao Nguyen Doan Pham, Evanston, IL (US)

(21) Appl. No.: 18/466,599

(22) Filed: Sep. 13, 2023

Related U.S. Application Data
(60) Provisional application No. 63/406,099, filed on Sep. 13, 2022.

(51) Int. Cl. C07D 231/56; A61P 35/00; C07D 401/12; C07D 405/12

(52) U.S. Cl. CPC C07D 231/56; A61P 35/00; C07D 401/12; C07D 405/12

(57) ABSTRACT

Disclosed are indazole compounds and derivatives thereof for use as modulators of the activity of mitogen-activated protein kinase 4 (MEK4). The disclosed compounds include 3-arylindazoles and 3-amino-indazoles which may be formulated in pharmaceutical composition for treating cell proliferative diseases and disorders associated with MEK4 activity, including cancer.

### CROSS-REFERENCE TO RELATED APPLICATIONS

This application claims benefit of priority to U.S. Application Ser. No. 63/406,099, filed Sep. 13, 2022, the contents of which are incorporated by reference in its entirety.

### STATEMENT REGARDING FEDERALLY SPONSORED RESEARCH OR DEVELOPMENT

This invention was made with government support under grant numbers CA070085, CA250353, CA255291, and CA217907 and GM105538 awarded by the National Institutes of Health. The government has certain rights in the invention.

### BACKGROUND

MEK4 is an upstream kinase in MAPK signaling pathways that phosphorylates p38 MAPK and JNK in response to mitogenic and cellular stress queues. MEK4 is overexpressed and induces metastasis in advanced prostate cancer lesions. However, the value of MEK4 as an oncology target has not been pharmacologically validated because selective chemical probes targeting MEK4 have not been developed.

To date, in the literature there has been minimal advancement in MEK4 inhibitor development. For example, HWY336, a protoberberine derivative, inhibits both MEK4 and MEK7. HWY336 not only has poor selectivity and only moderate potency, but the pharmacological parameters are not ideal as it is a tetracyclic alkaloid, a compounds class known for promiscuity in biological effects. Trihydroxyisoflavones have also been shown to have effects against MEK4 but not in a selective manner. For example, 7,3′,4′-trihydroxyisoflavone (THIF, 2) also inhibits Cot activity.

As such, there remains a need to develop selective and pharmacologically robust MEK4 inhibitors for treatment of cell proliferative diseases and disorders such as cancer.

### SUMMARY

Disclosed are indazole compounds and derivatives thereof for use as inhibitors of mitogen-activated protein kinase 4 (MEK4). Diseases and disorders treated by the disclosed compounds, pharmaceutical compositions, and methods may include, but are not limited to, cell proliferative diseases and disorders such as cancer.

The disclosed indazole compounds include 3-aryl substituted indazoles compounds, which may be referred to as “3-arylindzoles” and derivatives thereof. The disclosed indazole compounds may also include 3-amino substituted indazoles compounds, which may be referred to as “3-amino-indzoles” and derivatives thereof. The disclosed indazole compounds may also be substituted by 1,3-benzodioxolyl at the 3-position. The disclosed compounds may be described as having a Formula I as follows:

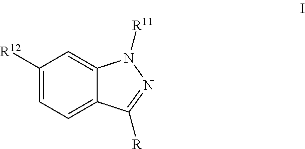

wherein
R is selected from the group consisting of —NR¹R², 1,3-benzodioxolyl, and aryl, wherein the aryl is optionally substituted with amino, carboxyl, —S(O)₂—NR³R⁴, —NR⁵—S(O)₂R⁶, or —NR⁷—C(O)R⁸;
R² is alkyl substituted with 1,3-benzodioxolyl or aryl, wherein the aryl is substituted with one or more hydroxyl, halo, or alkoxy;
R³ is selected from the group consisting of hydrogen, cycloalkyl, pyridyl, and aryl, wherein the aryl is optionally substituted with one or more substituents selected from the group consisting of halo, alkyl, haloalkyl, alkoxy, cyano, —S(O)₂-alkyl, —S(O)₂—NR⁹R¹⁰, —C(O)-alkyl, and —C(O)NR⁹R¹⁰;
R¹, R⁴, R⁵, R⁷, R⁹, and R¹⁰ are independently hydrogen or alkyl;
R⁶ is cycloalkyl;
R⁸ is selected from the group consisting of alkyl and alkenyl, wherein the alkenyl is optionally substituted with halo or amino;
R¹¹ is hydrogen, alkyl, or alkylaryl; and
R¹² is halo, alkyl, or haloalkyl.

Also contemplated herein are salts of the disclosed compounds including pharmaceutically acceptable salts of the disclosed compounds. Also contemplated are hydrates of the disclosed compounds including pharmaceutically acceptable hydrates of the disclosed compounds.

The disclosed compounds may have a Formula I(a):

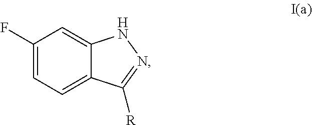

wherein R is the same group as defined in Formula (I).

The disclosed compounds may have a Formula I(a), wherein R is phenyl optionally substituted with amino, carboxyl, or —NHC(O)R⁸, and R⁸ is alkyl, vinyl, or propenyl, wherein the propenyl is substituted with halo or amino.

The disclosed compounds may have a Formula I(a), wherein R is —NHR², and R² is methyl substituted with 1,3-benzodioxolyl or phenyl, wherein the phenyl is substituted with one or more hydroxyl, halo, or alkoxy.

The disclosed compounds may have a Formula (a), wherein R is phenyl substituted with —NHS(O)₂R⁶ and R⁶ is C₃-C₅ cycloalkyl.

The disclosed compounds may have a Formula I(a), wherein R is phenyl substituted with —S(O)₂—NHR³, and R³ is selected from the group consisting of cycloalkyl, pyridyl, and phenyl, wherein the phenyl is optionally substituted with one or more substituents selected from the group consisting of halo, trifluoromethyl, alkoxy, cyano, —S(O)₂Me, —S(O)₂NH₂, acetyl, and —C(O)NH₂.

The disclosed compounds may be formulated as pharmaceutical compositions comprising the compounds or pharmaceutically acceptable salts or pharmaceutically acceptable hydrates thereof together with a pharmaceutically acceptable carrier, excipient, or diluent. The disclosed compounds and pharmaceutical compositions thereof may be utilized in treatment methods for a subject in need thereof. In particular, the disclosed compounds and pharmaceutical compositions thereof may be utilized to treat cell proliferative diseases or disorders such as cancer.

The disclosed compounds and pharmaceutical compositions may be utilized to treat a subject having a disease or disorder that is associated with MEK4 activity, such as a cell proliferative disease or disorder (e.g., cancer) that is associated with MEK4 activity in a subject in need thereof. For example, the disclosed compounds and pharmaceutical compositions may be administered to a subject in need thereof to treat the disease or disorder that is associated with MEK4 activity.

### BRIEF DESCRIPTION OF THE FIGURES

FIG. 1A shows a simplified MAPK signaling cascade.

FIG. 1B shows structures of MEK4 probes to date, distinguishing targeted MEK probe development campaigns.

FIG. 2A shows the proposed covalent inhibitor docked pose with Cys246 (inset: structure).

FIG. 2B illustrates the docked pose of 7 overlayed with 9c.

FIG. 2C shows SAR studies of covalent inhibitors and an extended linker between indazole and aryl ring.

FIG. 3A illustrates the docked pose of 10e, showing interactions with Met 181 and Ser 233, compared to the Glu 179 and Lys 187 that 7 interacts with.

FIG. 3B shows SAR studies of bioisosteres and sulfonamides.

FIG. 4A illustrates the docked pose of 13a.

FIG. 4B illustrates the docked pose of 14a.

FIG. 4C shows SAR studies of ‘reverse’ sulfonamides.

FIG. 4D illustrates the docked pose of 15o.

FIG. 4E shows a 2D view of 15o.

FIG. 5A shows selectivity against MEK7 for selected compounds.

FIG. 5B shows 10e and 1.5o selectivity against the MEK family at 1 μM and 10 μM.

FIGS. 6A-6M demonstrate that 15o and 10e inhibit JNK phosphorylation and exhibit anti-proliferative effects against pancreatic cancer cells. MEK1/2 inhibitors enhance the anti-proliferative effects of 15o and 10e in pancreatic cancer cells.

FIG. 6A shows that HEK293T cells were pretreated with anisomycin (3 μg/mL) for 45 minutes prior to the addition of 150 and 10e for 5 hours. The effects of 15o and 10e on JNK phosphorylation and total JNK expression levels were determined by western blotting was used as loading control. The blots are representative of at least three biological replicates.

FIGS. 6B and 6C show that human pancreatic cancer cells (CD18, MiaPaCa2) were pretreated with anisomycin (3 ng/mL) for 45 minutes prior to the addition of 150 and 10e for 5 hours. The effects of 15o and 10e on JNK phosphorylation and total JNK expression levels were determined by western blotting. HSP90 was used as loading control. The blots are representative of at least three biological replicates.

FIG. 6D demonstrates that HEK293T (˜30,000 cells) cells were treated with either DMSO or varying doses of 15o and 10e. After 72 hours, cells were trypsinized, counted, and the cell numbers were normalized against the DMSO treatment. Mean±SD. Graphs are representative of 3 biological replicates. One-way ANOVA; ns, non-significant **, p<0.01 ***, p<0.001.

FIGS. 6E and 6F show that human pancreatic cancer cells (˜30,000 cells) were treated with either DMSO or varying doses of 15o and 10e. After 72 hours, cells were trypsinized, counted, and the cell numbers were normalized against the DMSO treatment. Mean±SD. Graphs are representative of 3 biological replicates. One-way ANOVA; ns, non-significant ***, p<0.001 ****, p<0.0001.

FIG. 6G shows that HEK293T cells were pretreated with anisomycin (3 μg/mL) for 45 minutes prior to the addition of 15o (100 μM) and 10e (100 μM) for 5 hours. The effects of 150 and 10e on MEK7, phospho-p42/44 MAPK, total p42/44 MAPK, phospho-p38 MAPK, and total p38 MAPK expression levels were evaluated by western blotting. HSP90 was used as loading control. The blots are representative of at least three biological replicates.

FIGS. 6H and 6K show that human pancreatic cancer cells CD18 and MiaPaCa2 were pretreated with U0126 (1 μM or 5 μM) for 1 hour prior to the addition of MEK4 inhibitors (10 μM) for 5 hours. The effects of different treatments on phospho-p42/44 MAPK and total p42/44 MAPK expression levels were determined by western blotting. HSP90 was used as loading control. The blots are representative of at least three biological replicates.

FIGS. 6I and 6L show that human pancreatic cancer cells (˜30,000 cells) were treated with either DMSO, MEK1/2 inhibitor (U0126, 1 μM or 5 μM), MEK4 inhibitors (10 μM), or the combination of MEK1/2 and MEK4 inhibitors. After 72 hours, cells were trypsinized, counted, and the cell numbers were normalized against the DMSO treatment. Mean±SD. Graphs are representative of 3 biological replicates. Two-way ANOVA. *, p<0.05 **, p<0.01 ****, p<0.0001.

FIGS. 6J and 6M demonstrate Coefficient of Drug Interaction (CDI) calculations. Cell numbers from different treatments were normalized against the DMSO treatment, and CDI is calculated as follows: CDI=AB/(A×B), with AB being the ratio of the combination groups to control group and A or B being the ratio of the single agent group to the DMSO group. Mean±SEM from 3 biological replicates.

FIG. 7 lists the additional compounds made.

FIG. 8 shows the Eurofins Discovery ScanEDGE Kinase Panel for 15o.

FIG. 9 shows the Eurofins Discovery ScanELECT Kinase Panel with MEK3, 5, and 6. (NUCC-202952: 10e; NUCC-226297: 15o.)

FIG. 10A demonstrates the Log D studies conducted by BioDuro-Sundia.

FIG. 10B demonstrates the liver microsome stability studies conducted by BioDuro-Sundia.

FIG. 10C demonstrates the PAMPA studies conducted by BioDuro-Sundia.

FIG. 10D shows the Caco-2 studies of compounds 15o and 10e.

FIG. 10E shows the GYP inhibition studies of compounds 15o and 10 e.

### DETAILED DESCRIPTION

The present invention is described herein using several definitions, as set forth below and throughout the application.

### Definitions

The disclosed subject matter may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

A “subject in need thereof” as utilized herein refers to a subject in need of treatment for a disease or disorder associated with mitogen-activated protein kinase 4 (MEK4) activity. The term “subject” may be used interchangeably with the terms “individual” and “patient” and includes human and non-human mammalian subjects.

Diseases and disorders associated with MEK4 activity may include, but are not limited to, cell proliferative diseases and disorders such as cancer. Cancers may include, but are not limited to, adrenal cancer, anal cancer, bladder cancer, bone cancer, breast cancer, cervical cancer, Chronic Lymphocytic Leukemia (CLL), Chronic Myeloid Leukemia (CML), Chronic Myelomonocytic Leukemia (CMML), colorectal cancer, endometrial cancer, esophagus cancer, gallbladder cancer, carcinoid tumors, kidney cancer, leukemia, liver cancer, lung cancer, lymphoma, nasopharyngel cancer, non-small cell lung cancer, ovarian cancer, pancreatic cancer, penile cancer, pituitary tumors, prostate cancer, skin cancer, small cell lung cancer, small intestine cancer, stomach cancer, testicular cancer, thymus cancer, thyroid cancer, vaginal cancer, and vulvar cancer.

### Chemical Entities

New chemical entities and uses for chemical entities are disclosed herein. The chemical entities may be described using terminology known in the art and further discussed below.

As used herein, an asterisk “” or a plus sign “+” may be used to designate the point of attachment for any radical group or substituent group.

The term “alkyl” as contemplated herein includes a straight-chain or branched alkyl radical in all of its isomeric forms, such as a straight or branched group of 1-12, 1-10, or 1-6 carbon atoms, referred to herein as C₁-C₁₂ alkyl, C₁-C₁₀-alkyl, and C₆-alkyl, respectively.

The term “alkylene” refers to a diradical of straight-chain or branched alkyl group (i.e., a diradical of straight-chain or branched C₁-C₆ alkyl group). Exemplary alkylene groups include, but are not limited to —CH₂—, —CH₂CH₂—, —CH₂CH₂CH₂—, —CH(CH₃)CH₂—, —CH₂CH(CH₃)CH₂—, —CH(CH₂CH₃)CH₂—, and the like.

The term “halo” refers to a halogen atom or halogen radical (e.g., —F, —Cl, —Br, or —I).

The term “haloalkyl” refers to an alkyl group that is substituted with at least one halogen. For example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxy” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively.

The term “alkenyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido or carboxamido, amidino, amino, aryl, arylalkyl, azido, carbamate, carbonate, carboxy, carboxyamido, cyano, cycloalkyl, ester, ether, formyl, halo, haloalkyl, heteroaryl, heterocyclyl, hydroxyl, imino, ketone, nitro, phosphate, phosphonato, phosphinato, sulfate, sulfide, sulfonamide, sulfonyl or thiocarbonyl. In certain embodiments, the cycloalkyl group is not substituted, i.e., it is unsubstituted.

The term “cycloheteroalkyl” (or “heterocyclyl”) refers to a monovalent saturated cyclic, bicyclic, or bridged cyclic hydrocarbon group of 3-12, 3-8, 4-8, or 4-6 carbons in which at least one carbon of the cycloalkane is replaced with a heteroatom such as, for example, N, O, and/or S.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amino or carboxamido, carboxylic acid, —C(O)alkyl, —CO₂alkyl, carbonyl, carboxyl, carboxyamido, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The term “alkylaryl” refers to a radical containing an alkylene group, as defined herein, that is attached to an aryl group, as defined herein. One example of an alkylaryl group is benzyl.

The terms “heterocyclyl” and “heterocyclic group” are art-recognized and refer to saturated, partially unsaturated, or aromatic 3- to 1.0-membered ring structures, alternatively 3-to 7-membered rings, whose ring structures include one to four heteroatoms, such as nitrogen, oxygen, and sulfur. The number of ring atoms in the heterocyclyl group can be specified using x-y nomenclature where x and y are integers specifying the number of ring atoms. For example, a 3-7-membered heterocyclyl group refers to a saturated or partially unsaturated 3- to 7-membered ring structure containing one to four heteroatoms, such as nitrogen, oxygen, and sulfur. The designation “3-7-membered” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines (e.g., mono-substituted amines or di-substituted amines), wherein substituents may include, for example, alkyl, cycloalkyl, heterocyclyl, alkenyl, and aryl.

The terms “alkoxy” or “alkoxyl” are art-recognized and refer to an alkyl group, as defined above, haying an oxygen radical attached thereto. Representative alkoxy groups include methoxy, ethoxy, tert-butoxy and the like.

The term “carboxyl” as used herein refers to the radical —COON.

The term “hydroxyl” as used herein refers to the radical —OH.

The term “1,3-benzodioxolyl” as used herein refers to the radical

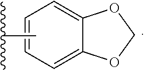

The term “pyridyl” as used herein refers to the radical

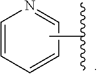

The term “vinyl” as used herein refers to the radical

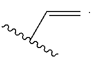 [SMILES: *C=C]

The term “propenyl” as used herein refers to the radical

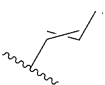

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” or “+” or “−” depending on the configuration of substituents around the stereogenic carbon atom and or the optical rotation observed. The present invention encompasses various stereo isomers of these compounds and mixtures thereof Stereoisomers include enantiomers and diastereomers. Mixtures of enantiomers or diastereomers may be designated (±)” in nomenclature, but the skilled artisan will recognize that a structure may denote a chiral center implicitly. It is understood that graphical depictions of chemical structures, e.g., generic chemical structures, encompass stereoisomeric forms of the specified compounds, unless indicated otherwise. Also contemplated herein are compositions comprising, consisting essentially of, or consisting of an enantiopure compound, which composition may comprise, consist essential of, or consist of at least about 50%, 60%, 70%, 80%, 90%, 95%, 96%, 97%, 98%, 99%, or 100% of a single enantiomer of a given compound (e.g., at least about 99% of an R enantiomer of a given compound).

The substituents on the aryl (e.g. phenyl) rings in the compounds of the disclosure may be at ortho-, meta-, or para-positions.

### Use of the Disclosed Compounds for Modulating Mitogen-Activated Protein Kinase 4 (MEK4) Activity

The disclosed compounds may exhibit one or more biological activities. In some embodiments, the disclosed compounds may modulate the activity of mitogen-activated protein kinase 4 (MEK4). For example, in some embodiments, the disclosed compounds inhibit the activity of MEK4 by at least 50%, 60%, 70%, 80%, 90%, 95%, 96%, 97%, 98%, or 99% at a concentration of less than about 100 μM, 50 μM, 10 μM, 1 μM, 0.1 μM, 0.05 μM, 0.01 μM, 0.005 μM, 0.001 μM, or less. The disclosed compounds may inhibit the growth of cells that express MEK4 (preferably by at least 50%, 60%, 70%, 80%, 90%, 95%, 96%, 97%, 98%, or 99% at a concentration of less than about 100 μM, 50 μM, 10 μM, 1 μM, 0.1 μM, 0.05 μM, 0.01 μM, 0.00.5 μM, 0.001 μM, or less). The disclosed compounds may not inhibit the growth of cells that do not express MEK4 (preferably by not more than 50%, 40%, 30%, 20%, 10%, 5%, 4%, 3%, 2% or less at a concentration of greater than about 0.001 μM, 0.005 μM, 0.01 μM, 0.5 μM, 0.1 μM, 1.0 μM, 10 μM, and 100 μM or higher). Concentration ranges also are contemplated herein, for example, a concentration range bounded by end-point concentrations selected from 0.001 μM, 0.005 μM, 0.01 μM, 0.5 μM, 0.1 μM, 1.0 μM, 10 μM, and 100 μM.

Cell proliferation and inhibition thereof by the presently disclosed compounds may be assessed by cell viability methods disclosed in the art including colorimetric assays that utilize dyes such as MTT, XTT, and MTS to assess cell viability. Preferably, the disclosed compounds have an IC₅₀ of less than about 10 μM, 5 μM, 1 μM, 0.5 μM, 0.01 μM, 0.005 μM, 0.001 μM or lower in the selected assay.

Pharmaceutical Compositions, Formulations, and Methods of Treatment for Diseases and Disorders Associated with Mitogen-Activated Protein Kinase 4 (MEK4) Activity

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage pit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

In some embodiments, the compounds disclosed herein may be formulated as pharmaceutical compositions that include: (a) a therapeutically effective amount of one or more compounds as disclosed herein; and (b) one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to about 1000 mg/kg body weight (preferably about 0.5 to about 500 mg/kg body weight, more preferably about 50 to about 100 mg/kg body weight). In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 μM, 0.005 μM, 0.01 μM, 0.5 μM, 0.1 μM, 1.0 μM, 10 μM, and 100 μM (e.g., 0.1 μM-1.0 μM).

The disclosed compounds and pharmaceutical compositions comprising the disclosed compounds may be administered in methods of treating a subject in need thereof For example, in the methods of treatment a subject in need thereof may include a subject having a cell proliferative disease, disorder, or condition such as cancer.

In some embodiments of the disclosed treatment methods, the subject may be administered a dose of a compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as end-points any of these disclosed doses (e.g., 2.5 mg-200 mg).

In some embodiments of the disclosed treatment methods, a minimal dose level of a compound tier achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. In some embodiments, a maximal dose level of a compound for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. Minimal and/or maximal dose levels of the compounds for achieving therapy in the disclosed methods of treatment may include dose levels falling within ranges having as end-points any of these disclosed dose levels (e.g., 500-2000 ng/kg body weight of the subject).

The compounds for use according to the methods of disclosed herein may be administered as a single compound or a combination of compounds. For example, a compound that modulates MEK4 activity may be administered as a single compound or in combination with another compound that modulates MEK4 activity or that has a different pharmacological activity.

As indicated above, pharmaceutically acceptable salts of the compounds are contemplated and also may be utilized in the disclosed methods. The term “pharmaceutically acceptable salt” as used herein, refers to salts of the compounds which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion fowling a part of any salt of a compound disclosed herein is may not be critical to the activity of the compound, so long as the salt as a whole is pharmacologically acceptable and as long as the counterion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of treating a disease or disorder associated MEK4 activity. As used herein, the terms “treating or to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with MEK4 activity.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein, Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### 3-Arylindazoles and 3-Amino-Indazoles as Selective Inhibitors of Mitogen-Activated Kinase 4 (MEK4)

The subject matter disclosed herein relates to indazole compounds and uses thereof. Particularly disclosed are 3-ayrlindazole compounds, 3-amino-indazole compounds, and indazole compounds substituted with 1,3-benzodioxolyl, optionally which may be substituted at one or more positions, and the use thereof for modulating the activity of kinases such as mitogen-activated kinase 4 (MEK4) in a subject in need thereof.

In some embodiments, the disclosed compounds may have Formula I:

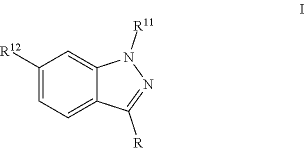

wherein
R is selected from the group consisting of —NR′R¹R², 1,3-benzodioxolyl, and aryl, wherein the aryl is optionally substituted with amino, carboxyl, —S(O)₂—NR³R⁴, —NR⁵—S(O)₂R⁶, or —NR⁷—C(O)R⁸;
R² is alkyl substituted with 1,3-benzodioxolyl or aryl, wherein the aryl substituted with one or more hydroxyl, halo, or alkoxy;
R³ is selected from the group consisting of hydrogen, cycloalkyl, pyridyl, and aryl wherein the aryl is optionally substituted with one or more substituents selected from the group consisting of halo, alkyl, haloalkyl, alkoxy, cyano, —S(O)₂-alkyl, —S(O)₂—NR⁹R¹⁰, —C(O)-alkyl, and —C(O)NR⁹R¹⁰;
R¹, R⁴, R⁵, R⁷, R⁹, and R¹⁰ are independently hydrogen or alkyl;
R⁶ is cycloalkyl;
R⁸ is selected from the group consisting of alkyl and alkenyl, wherein the alkenyl is optionally substituted with halo or amino;
R¹¹ is hydrogen, alkyl, or alkylaryl; and
R¹² is halo, alkyl, or haloalkyl.

In some embodiments, the compound has a Formula I(a):

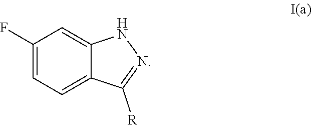

In some embodiments, R is phenyl optionally substituted with amino, carboxyl, or —NHC(O)R⁸, and R⁸ is alkyl, vinyl, or propenyl, wherein the propenyl is substituted with halo or amino in compounds of Formula I(a).

In such embodiments, the compound is

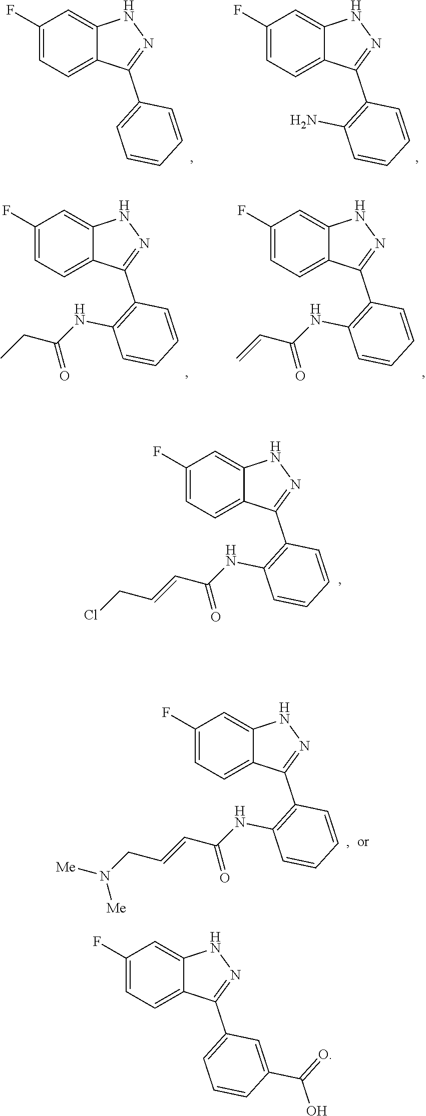

In some embodiments, R is —NHR², and R² is methyl substituted with 1,3-benzodioxolyl or phenyl, wherein the phenyl is substituted with one or more hydroxyl, halo, or alkoxy in compounds of Formula 1(a).

In such embodiments, the compound is

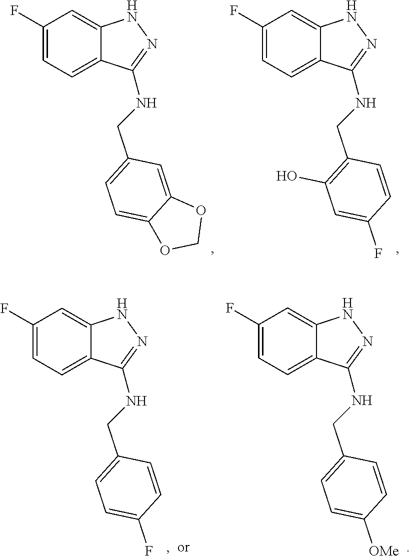 [SMILES: Fc1ccc2c(NCc3ccc4c(c3)OCO4)n[nH]c2c1 ; Oc1cc(F)ccc1CNc1n[nH]c2cc(F)ccc12 ; Fc1ccc(CNc2n[nH]c3cc(F)ccc23)cc1 ; COc1ccc(CNc2n[nH]c3cc(F)ccc23)cc1]

In some embodiments, R is phenyl substituted with —NHS(O)₂R⁶, and R⁶ is C₃-C₅ cycloalkyl in compounds of Formula 1(a).

In such embodiments, the compound is

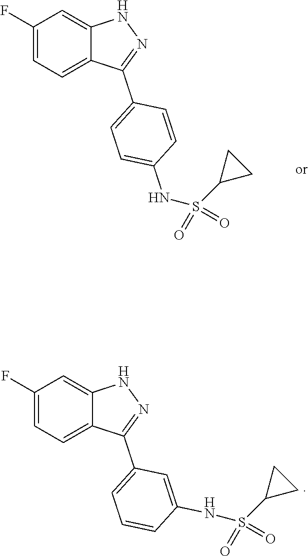 [SMILES: O=S(=O)(Nc1ccc(-c2n[nH]c3cc(F)ccc23)cc1)C1CC1 ; O=S(=O)(Nc1cccc(-c2n[nH]c3cc(F)ccc23)c1)C1CC1]

In some embodiments, R is phenyl substituted with —S(O)₂—NHR³, and R³ is selected from the group consisting of cycloalkyl, pyridyl, and phenyl, wherein the phenyl is optionally substituted with one or more substituents selected from the group consisting of halo, trifluoromethyl, alkoxy, alkyl, cyano, —S(O)₂Me, —S(O)₂NH₂, acetyl, and —C(O)NH₂ in compounds of Formula I.

In such embodiments, the compound is

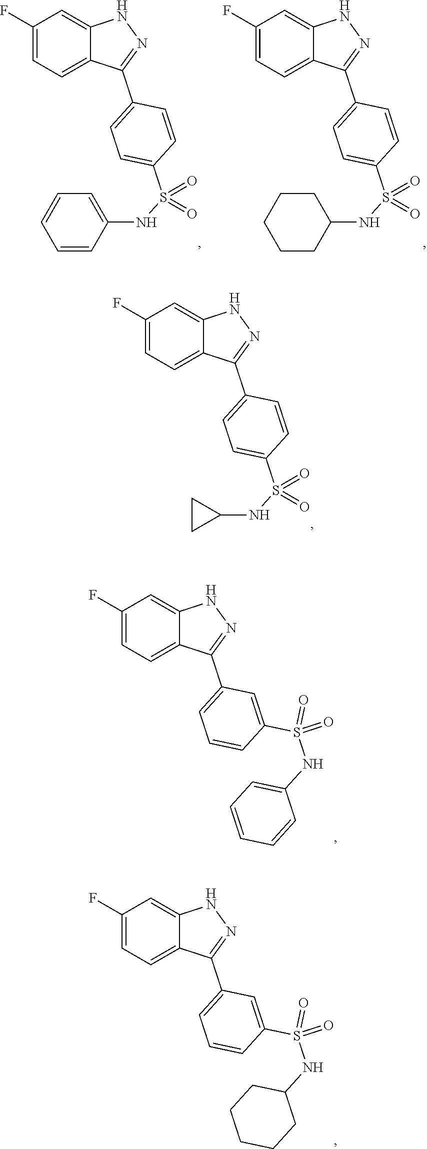 [SMILES: O=S(=O)(Nc1ccccc1)c1ccc(-c2n[nH]c3cc(F)ccc23)cc1 ; O=S(=O)(NC1CCCCC1)c1ccc(-c2n[nH]c3cc(F)ccc23)cc1 ; O=S(=O)(NC1CC1)c1ccc(-c2n[nH]c3cc(F)ccc23)cc1 ; O=S(=O)(Nc1ccccc1)c1cccc(-c2n[nH]c3cc(F)ccc23)c1 ; O=S(=O)(NC1CCCCC1)c1cccc(-c2n[nH]c3cc(F)ccc23)c1]

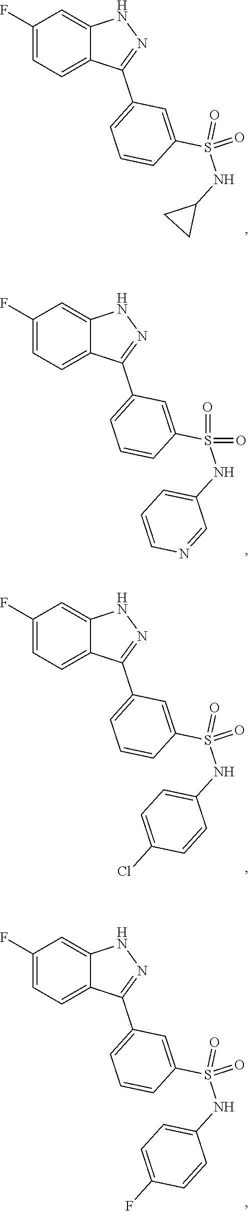 [SMILES: O=S(=O)(NC1CC1)c1cccc(-c2n[nH]c3cc(F)ccc23)c1 ; O=S(=O)(Nc1cccnc1)c1cccc(-c2n[nH]c3cc(F)ccc23)c1 ; O=S(=O)(Nc1ccc(Cl)cc1)c1cccc(-c2n[nH]c3cc(F)ccc23)c1 ; O=S(=O)(Nc1ccc(F)cc1)c1cccc(-c2n[nH]c3cc(F)ccc23)c1]

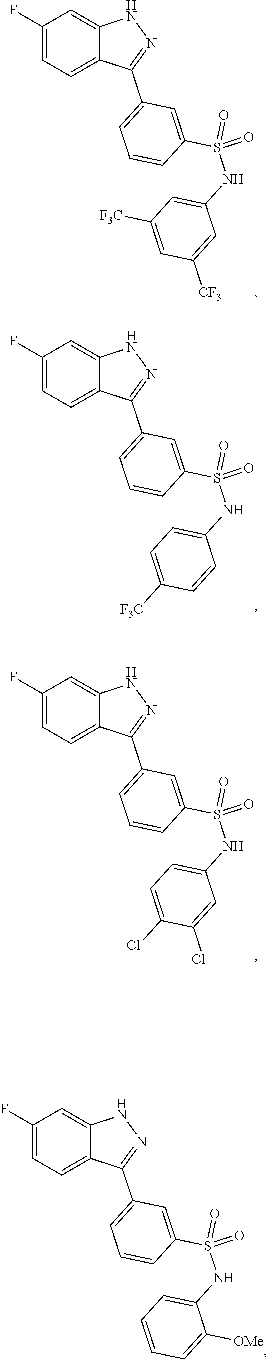 [SMILES: O=S(=O)(Nc1cc(C(F)(F)F)cc(C(F)(F)F)c1)c1cccc(-c2n[nH]c3cc(F)ccc23)c1 ; O=S(=O)(Nc1ccc(C(F)(F)F)cc1)c1cccc(-c2n[nH]c3cc(F)ccc23)c1 ; O=S(=O)(Nc1ccc(Cl)c(Cl)c1)c1cccc(-c2n[nH]c3cc(F)ccc23)c1 ; COc1ccccc1NS(=O)(=O)c1cccc(-c2n[nH]c3cc(F)ccc23)c1]

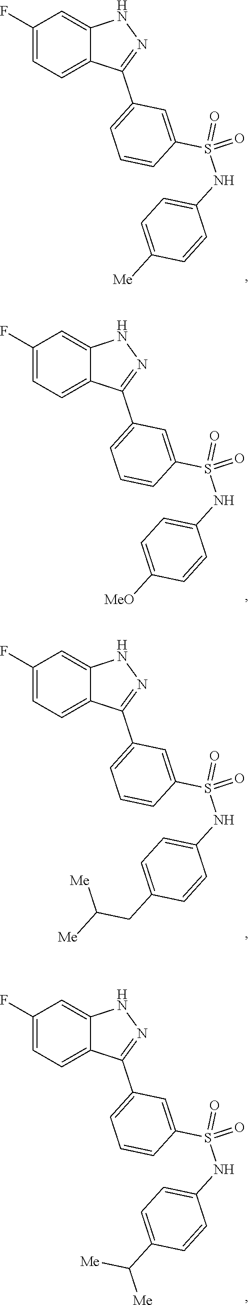 [SMILES: Cc1ccc(NS(=O)(=O)c2cccc(-c3n[nH]c4cc(F)ccc34)c2)cc1 ; COc1ccc(NS(=O)(=O)c2cccc(-c3n[nH]c4cc(F)ccc34)c2)cc1 ; CC(C)Cc1ccc(NS(=O)(=O)c2cccc(-c3n[nH]c4cc(F)ccc34)c2)cc1 ; CC(C)c1ccc(NS(=O)(=O)c2cccc(-c3n[nH]c4cc(F)ccc34)c2)cc1]

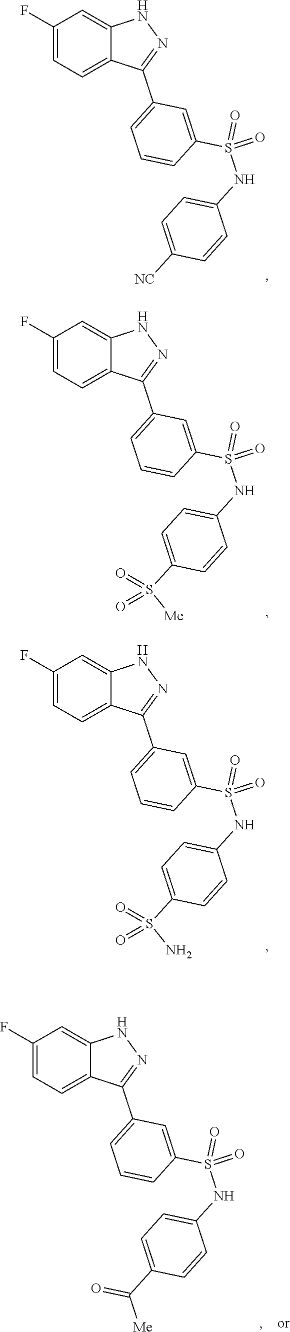 [SMILES: N#Cc1ccc(NS(=O)(=O)c2cccc(-c3n[nH]c4cc(F)ccc34)c2)cc1 ; CS(=O)(=O)c1ccc(NS(=O)(=O)c2cccc(-c3n[nH]c4cc(F)ccc34)c2)cc1 ; NS(=O)(=O)c1ccc(NS(=O)(=O)c2cccc(-c3n[nH]c4cc(F)ccc34)c2)cc1 ; CC(=O)c1ccc(NS(=O)(=O)c2cccc(-c3n[nH]c4cc(F)ccc34)c2)cc1]

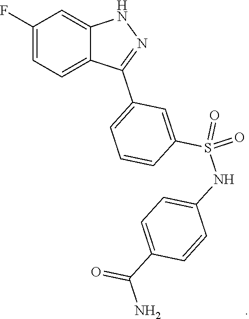 [SMILES: NC(=O)c1ccc(NS(=O)(=O)c2cccc(-c3n[nH]c4cc(F)ccc34)c2)cc1]

In some embodiments, the compound is

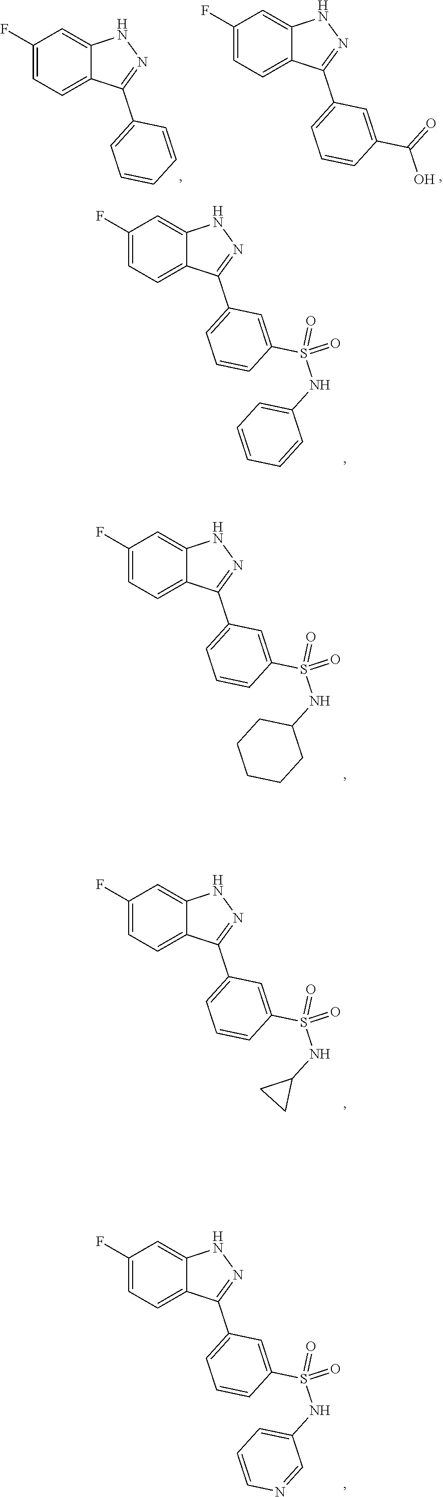

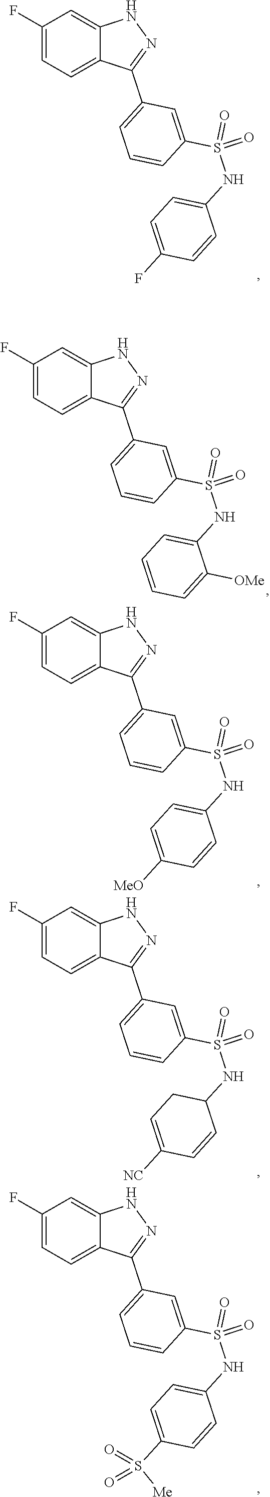

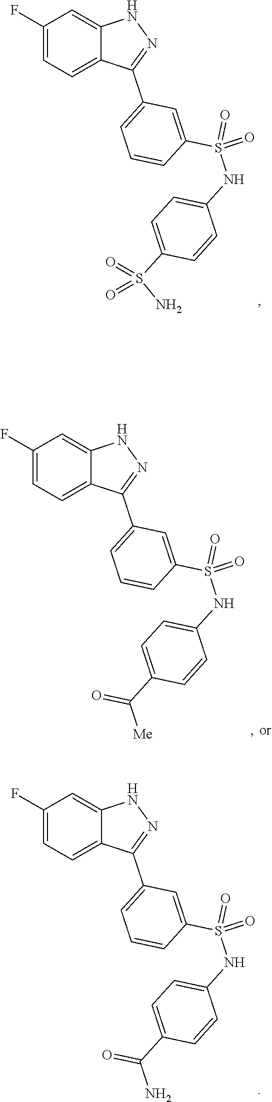 [SMILES: NS(=O)(=O)c1ccc(NS(=O)(=O)c2cccc(-c3n[nH]c4cc(F)ccc34)c2)cc1 ; CC(=O)c1ccc(NS(=O)(=O)c2cccc(-c3n[nH]c4cc(F)ccc34)c2)cc1 ; NC(=O)c1ccc(NS(=O)(=O)c2cccc(-c3n[nH]c4cc(F)ccc34)c2)cc1]

In such embodiments, the compound is

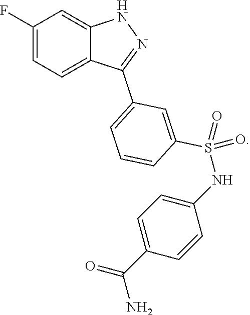 [SMILES: NC(=O)c1ccc(NS(=O)(=O)c2cccc(-c3n[nH]c4cc(F)ccc34)c2)cc1]

In some embodiments, the compound is

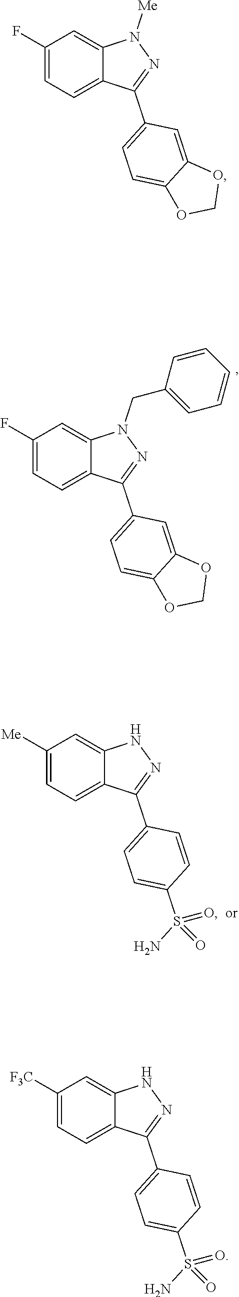

The disclosed compounds, salts thereof, and/or hydrates thereof may be formulated as pharmaceutical compositions comprising the compounds, salts thereof, and/or hydrates thereof, in a pharmaceutically acceptable carrier. The pharmaceutical compositions may be formulated for treating diseases or disorders associated with mitogen-activated protein kinase 4 (MEK4) activity in a subject in need thereof.

In some embodiments, the disclosed compounds and pharmaceutical compositions may be utilized for treating a subject having a cell proliferative disease, disorder, such as prostate, breast or pancreatic cancer associated with mitogen-activated protein kinase 4 (MEK4) activity, the method comprising administering to the subject the compounds and/or the pharmaceutical compositions. In these methods, the subject may be administered an amount of the compound sufficient to modulate MEK4 activity.

In such embodiments, the disease or disorder is a cell proliferative disease or disorder such as cancer. In some such embodiments, the cancer is selected from the group consisting of prostate cancer, breast cancer, pancreatic cancer, colon cancer, and combinations thereof. In some such embodiments, the cancer is pancreatic cancer.

### EXAMPLES

The following Examples are illustrative and should not be interpreted to limit the scope of the claimed subject matter.

### Example I

Growth, division, and development of healthy cells relies on efficient response to environmental survival cues. The conserved mitogen-activated protein kinase (MAPK) family of pathways interface extracellular stimuli to intracellular processes for this purpose. MEK4 inhibitors demonstrating significant reduction of phospho-JNK and anti-proliferative properties against pancreatic cancer cell lines are described herein. Furthermore, molecular inhibition of MEK4 pathway activates the MEK1/2 pathway, with the combination of MEK1/2 and MEK4 inhibitors demonstrating synergistic effects against pancreatic cancer cells. The inhibitors described herein provided insight into the crosstalk between MAPK pathways and tools for elucidating the roles of MEK4 in disease states, findings which will pave the way for better understanding of the MAPK pathways and development of additional probes.

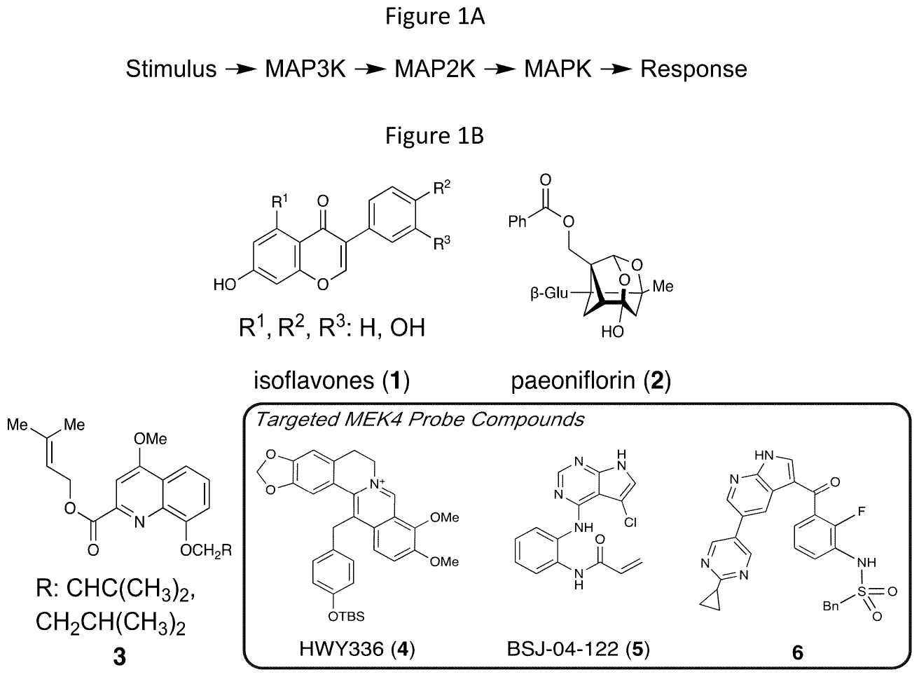

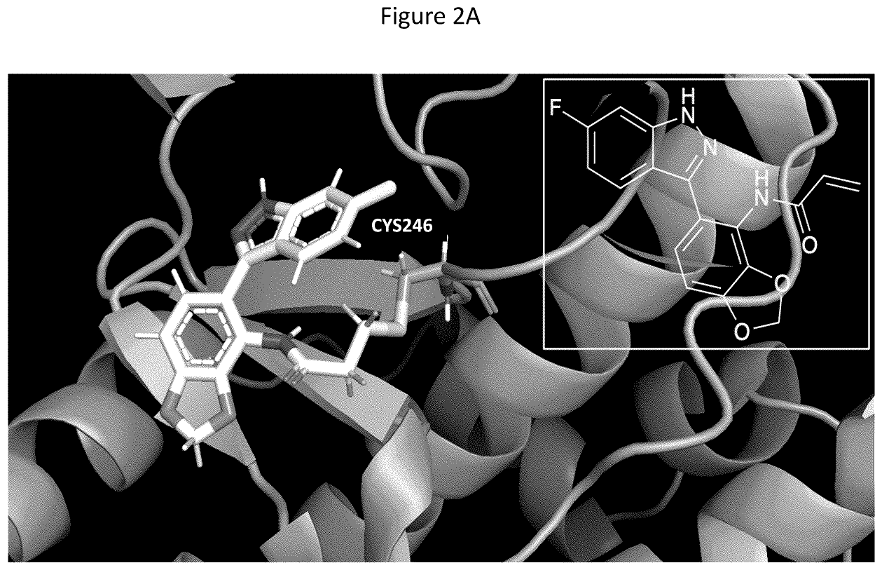

The mitogen-activated protein kinase (MAPK) signal cascades are ubiquitous across most organisms and responsible for essential cellular processes such as cell growth, division, and death.¹⁻³ Within the MAPK signaling pathways, the MAPK/ERK (MEK or MAP2K) family of kinases are intermediary proteins that directly activate the MAP kinases responsible for cellular response (FIG. 1A).⁴ Of the seven kinases in this family, MEK1 and MEK2 were the first to be discovered and thoroughly studied, ultimately giving rise to four FDA-approved MEK1/2 inhibitors for single-agent or combination therapies for various melanomas and neurofibromatosis type 1.⁵⁻⁷ In particular, MEK4 plays a role in both essential and detrimental capacities. There is evidence that in early stages of development, MEK4 is vital for proper maturation of the liver and the immune system.⁹ However, overexpression of MEK4 has been observed in cancers of the pancreas and prostate, which have some of the lowest survival rates among the various cancers.¹⁰ Additionally, MEK4 has been implicated in neurological conditions including stroke, Parkinson's, Huntington's, and Alzheimer's disease, as well as cardiac hypertrophy.¹¹

Currently, there are seven reported MEK4 inhibitors. (FIG. 1B) Three inhibitors (1-3) were discovered through a top-down approach, in which they were found to positively affect disease models, and their mechanism of action was later determined to be MEK4 inhibition.¹²⁻¹⁴ As these compounds are mostly derived from natural products, the use and further development of these compounds can be challenging. Additionally, the potency and selectivity profile of these compounds are usually not optimal, and many compounds are either not tested for selectivity or found to have significant off-target effects. HWY336 (4), developed by Song and coworkers in 2014, was the first report of a ‘targeted’ MEK4 inhibitor campaign.¹⁵ However, HWY336 displayed only modest potency and poor selectivity. Compound 5 (FIG. 1B) did not have great selectivity for MEK4 over MEK7. Compound 6 was not evaluated for activity or in cellular models. Given the gap in knowledge regarding the molecular mechanisms surrounding MEK4 and the small number of MEK4 probes available, there is a need for continued development of potent and selective MEK4 inhibitors.

The development of indazole-derived inhibitors are described herein, as an addition to the existing tools available for studying MEK4. SAR studies were performed to improve cellular permeability while maintaining potency. Additional studies provided preliminary evidence of the synergistic effects of co-dosing with MEK1/2 inhibitors, in addition to revealing crosstalk relationships across the two MAPK pathways.

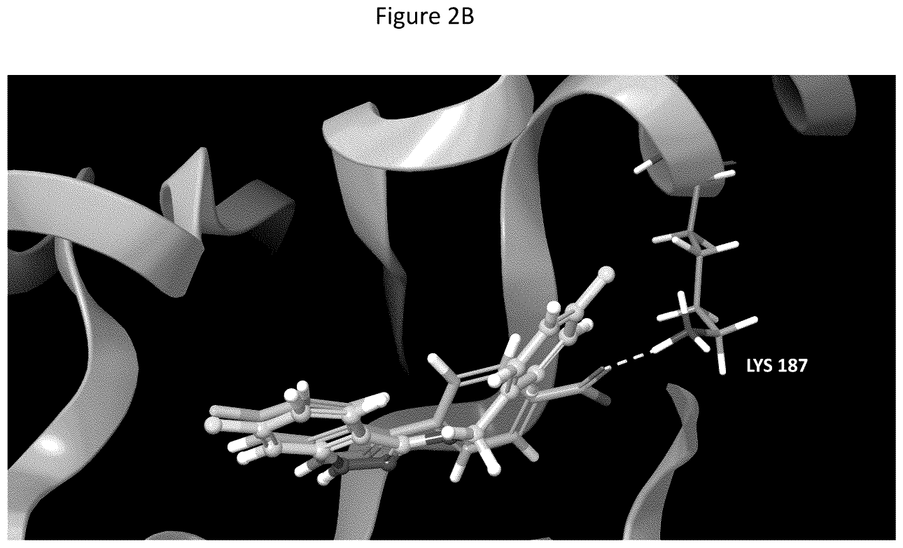

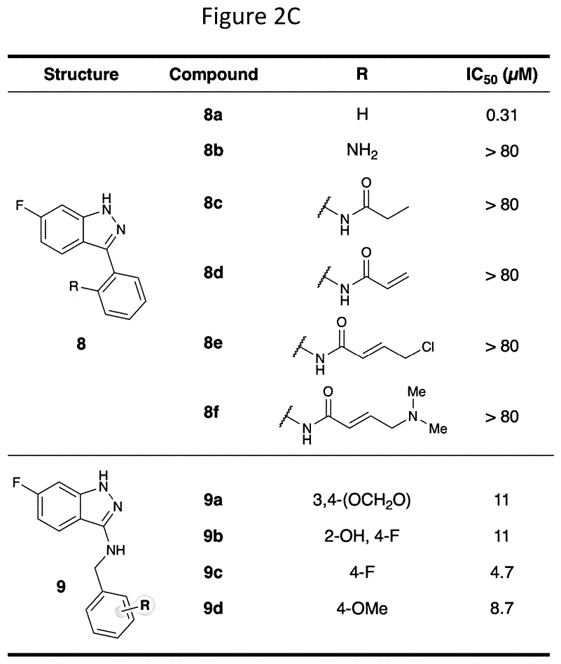

Covalent inhibitors were synthesized with guidance provided by computational modeling. Covalent binding can prevent dissociation from the protein and maintain potency even if the binding site is more shallow. Additionally, by targeting cysteines that are unique to the protein, selectivity can also be improved. The generated computational pose suggested that the analog would be able to react covalently with Cys246 (FIG. 2A).

All compounds were evaluated using the ADP-Glo assay, an assay with readouts that utilizes ATP to measure the functional inhibition of activity, following protocols in the supporting information.^{19, 24-25} Compound 8a had an IC₅₀ value of 0.31 μM. Through an amine in the or position on the phenyl ring, three variants of conjugate acceptors with varying reactivity were installed, as well as a non-covalent control. Compounds 8b-8f have IC₅₀ values of greater than 80 μM (FIG. 2C), indicating that nucleophilic addition was not occurring. The hydrophilic substituents al the ortho position may experience repulsion from hydrophobic residues in that area of the pocket, resulting in a decrease in potency.

Non-covalent inhibitors and compounds with an elongated linkage between the indazole and the aryl substituent were synthesized. This type of compound might be able to reach more residues or strengthen existing interactions with residue contacts (FIG. 2B). Various benzylamine derivatives were installed on the indazole and compounds 9a-9d, which have micromolar potency, were synthesized. Based on the computationally docked pose of 8d (FIG. 2B), the phenyl rings of these compounds were interacting negatively with Lys187. Observations of the model indicated that elongation of the linkage between the indazole and the aryl ring created compounds that might not fit in the binding site, and further designs would not improve potency. Additional substitutions at the indazole nitrogen furnished compounds S2-3 (FIG. 7). The model indicated that these substitutions could prevent the indazole nitrogen from interacting with residue Glu179 at the back of the binding pocket.

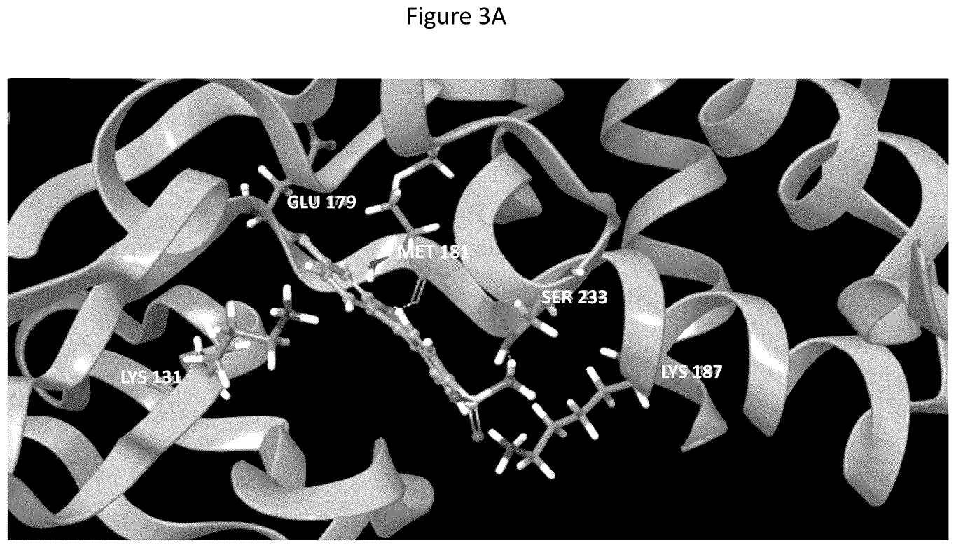

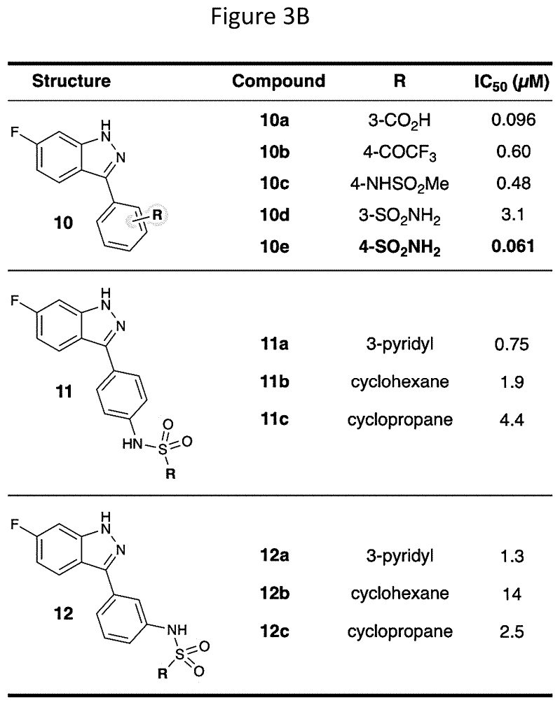

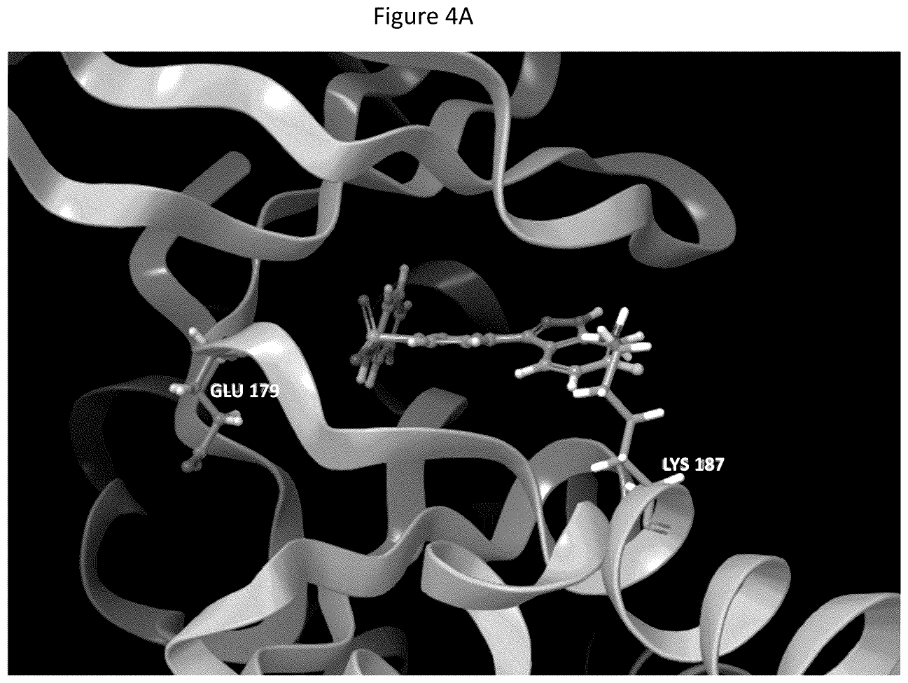

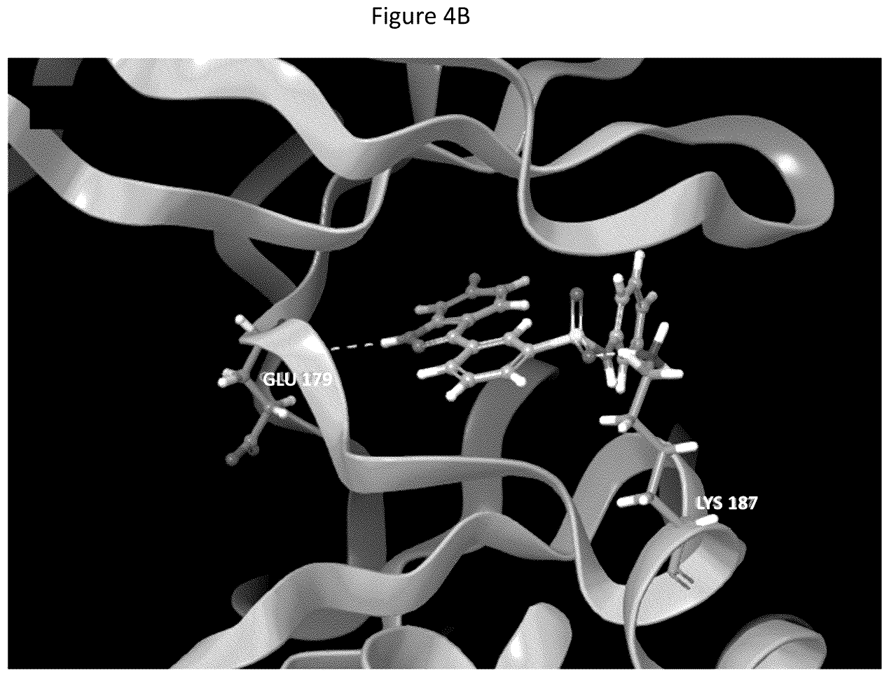

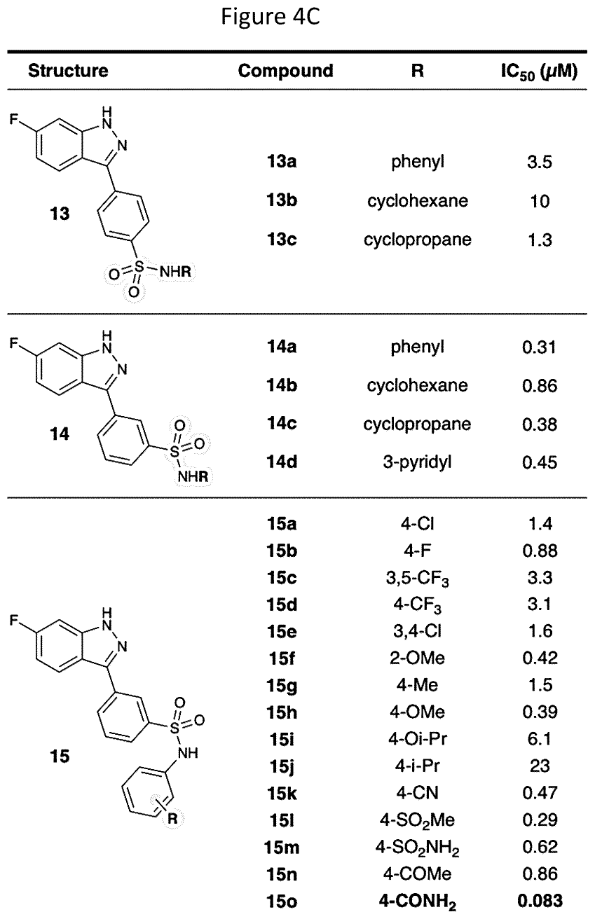

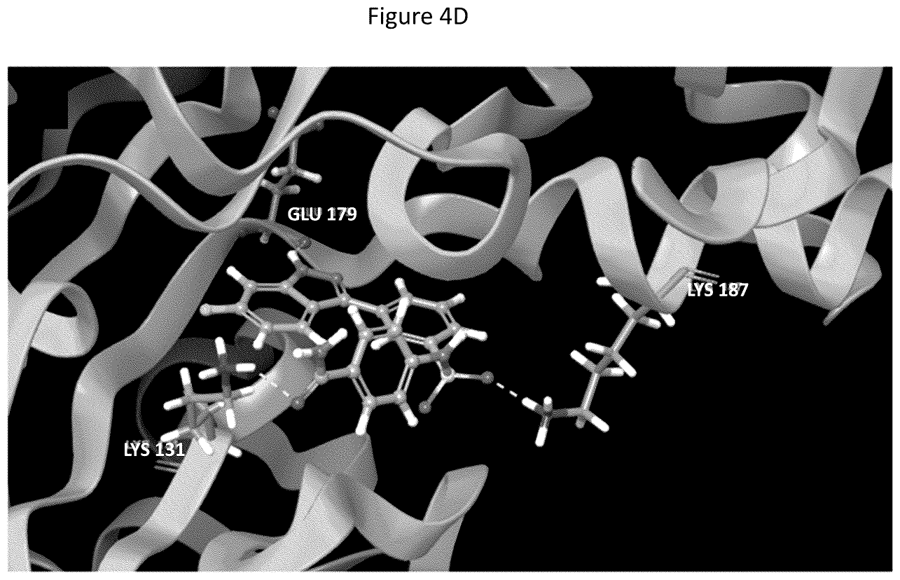

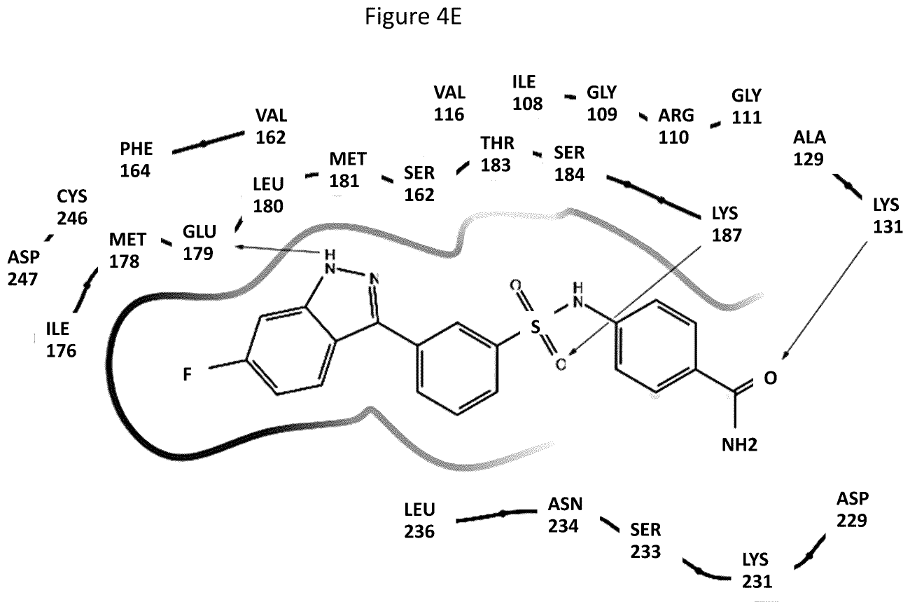

Restoring the original indazole-aryl linkage, other alterations were attempted. Compound 10 of FIG. 3B was synthesized. More lipophilic compounds with similar ratios of hydrogen bond donors and acceptors were synthesized to retain contact with Lys187 (compounds 10a-e, FIG. 3B). Most analogs of 10 had sub-micromolar IC₅₀ values, with sulfonamide 10e having an IC₅₀ value of 0.061 μM. The modeled pose of 10e showed the indazole still interacting with the back of the binding pocket, and the terminal primary sulfonamide interacting with a polar residue. However, instead of interacting with residues Glu179 and Lys187 like 7 does, 10e appears to interact with the backbone of Met181 and residue Ser233 in our model, creating a pose that sits shallower in the pocket (FIG. 3A). The potency of 10e can be attributed to the similarity in the computationally observed binding pose. Modification of the fluoro group on the indazole of 10e yield compounds S4-5 of FIG. 7. Further modifications were made by adding substituents to the sulfonamide of 10e. Analogs 11-12 were the first compounds synthesized with the sulfonamide handle. Notably, these compounds were first made with the nitrogen of the sulfonamide closer to the aryl ring, followed by the sulfonyl group. This synthetic route was designed based on the commercially available borylated anilines and wide variety of sulfonyl chlorides that would allow facile access to a diverse library of compounds. None of the analogs performed better than 10e (FIG. 3B). Alteration by installing the sulfonyl closer to the aryl ring was made to mimic the regiochemistry of compounds 13-14 (FIG. 4C).

The para-substituted analogs (13) were outperformed by the meta-substituted analogs (14). When analogs 13a and 14a were docked into our model, the para-substituted compound poses did not include the key indazole ring interaction with the back of the binding pocket or Glu179 (FIG. 4A). Instead, the computationally generated pose featured the aryl ring towards the back of the binding pocket. Additionally, the pose of 13a did not appear to have any other residue interactions with the binding pocket. In comparison, the binding pose of 14a showed the sulfonyl group interacting with Lys187, which might contribute to the higher potency (FIG. 4B). The major differences in interactions with the binding pocket might be the source of the ˜10-fold difference in potency. The binding pose of 14a led to the expansion of this scaffold into a series of analogs, Among the early compounds of this series (FIG. 4C, 14a-d,), the phenyl derivative performed the best and was the most synthetically tractable derivative to expand from. With the phenyl ring as the scaffold, the compound library was diversified to include functional groups such as halogens, alkyl groups, and heteroatom-containing functional groups at various positions on the ring (compounds 15a-o, FIG. 4C). The heteroatom-containing functional groups (compounds 15f, h, i, k-o) performed the best, and the para-primary amide, 15o was the most potent of the series with an IC₅₀ value of 0.083 μM. This might be attributed to the new interaction between the primary amide of 15o and Lys131, in addition to the previously identified interactions in our MEK4 model between Glu179 and the indazole and between Lys187 and the meta-sulfonamide (FIGS. 4D & 4E).

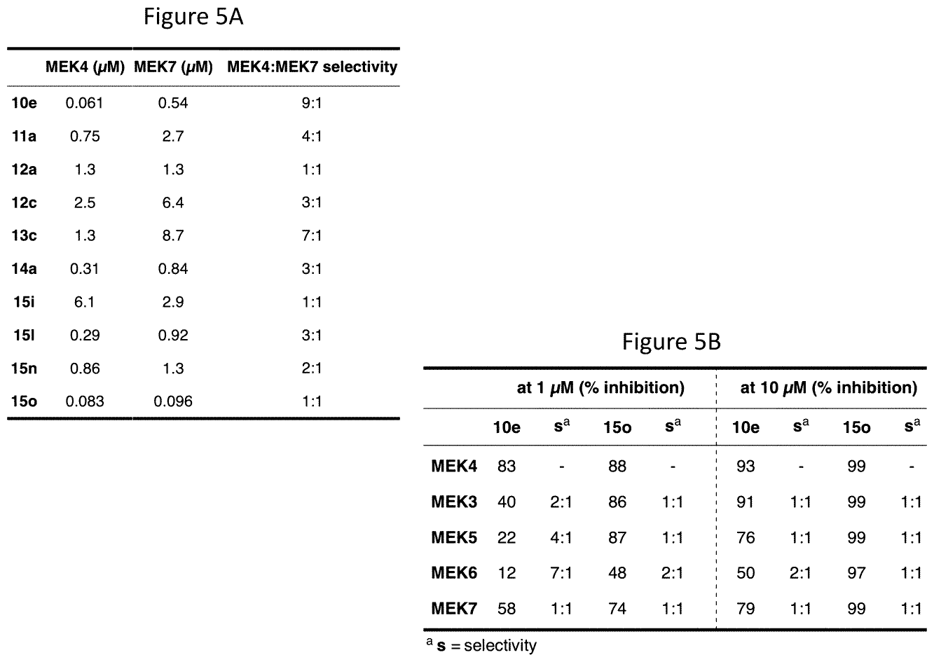

The compounds were then evaluated both in vitro and in a cellular system. When studying kinase inhibitors, there are a multitude of ways to evaluate selectivity across kinases. As a first point of evaluation, a focused library of 10 compounds, with analogs from each series of compounds, was tested against MEK7 in the ADP-Glo functional assay (FIG. 5A).^{19, 24-25} Ratios ranged from slim margins to almost 10-fold selectivity for MEK4. In general, the selectivity decreased as larger substituents were added to the aryl ring.

To compare our compounds to the rest of the MEK inhibitors discovered thus far, we turned to the DiscoverX platform (KINOMEscan®, Eurofins DiscoverX), a widely used assay for comparing selectivity across the kinome. The DiscoverX platform reports the thermodynamic K_{d} in order to facilitate direct comparison of inhibitor affinity across the kinome, independent of ATP concentration. To test for selectivity across the MEK family, we submitted 10e and 15o for testing using the scanELECT® DiscoverX platform. We found that with the addition of the extra phenyl ring in 15o compared to 10e, the overall selectivity against the MEK family decreased. Nevertheless, 15o maintained a slim margin of selectivity towards MEK4, and 10e showed larger margins of selectivity towards MEK4 (FIG. 5B). We then subjected 15o to a kinome-wide scan using the scanEDGE® DiscoverX platform. To ensure we observed all potential interactions with other kinases across the kinome and matched our cellular studies, we tested 15o against 97 kinases at a concentration of 10 μM. 15o hit 28 out of 97 kinases at 80% or higher inhibition, The main classes of kinases affected were tyrosine-like kinases (TK), tyrosine kinase-like kinases (TKL), serine threonine kinases (STE), cyclin-dependent kinases, mitogen-activated protein kinases, glycogen synthase kinases and CDK-like kinases (all encompassed within ‘CMGC’). Notably, it did not inhibit related kinases ERK1, BRAF or RAF, though it did inhibit downstream kinases JNK1/2/3.

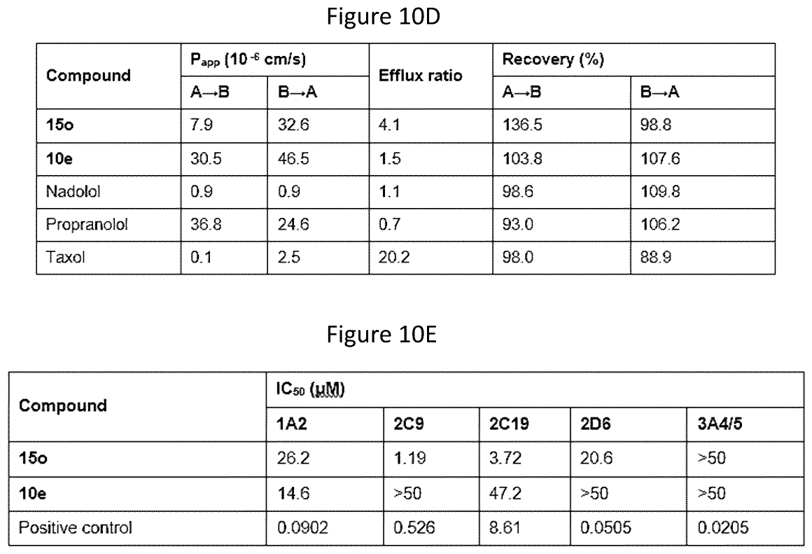

Additionally, we sought to evaluate some standard pharmacokinetic properties of the synthesized compounds, preceding their potential use as probe compounds in in vivo studies (FIGS. 10A-10E). At a pH of 7.4, the log D of 15o was determined to be 3.22, while the log D of 10e was determined to be 2.80 (FIG. 10A). Both compounds exhibited good stability in the microsome assay, with 15o having a half-life of greater than 185 minutes, and 10e having a half-life of 114 minutes (FIG. 10B). In the PAMPA assay, 15o exhibited low permeability, while 10e performed slightly better (FIG. 10C). The Caco-2 permeability assay revealed that while both compounds showed medium to high permeability, only 15o was likely to be a Pgp substrate (FIG. 10D). Lastly, 10e showed a better selectivity profile than 15o in relation to CYP inhibition (FIG. 10E). While both compounds did not inhibit CYP isoforms 3A4/5, 10e also avoided inhibiting both 2D6 and 2C9. 15o exhibited low inhibition of 2D6 while inhibiting 2C9 to a higher degree. Both compounds inhibited CYP isoforms 1A2 and 2C19 to varying degrees.

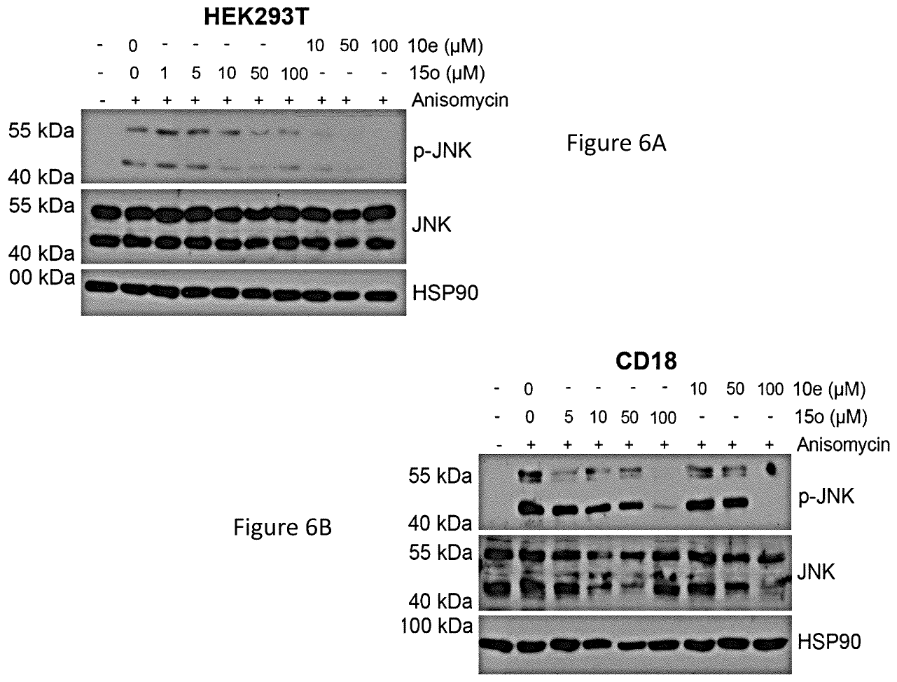

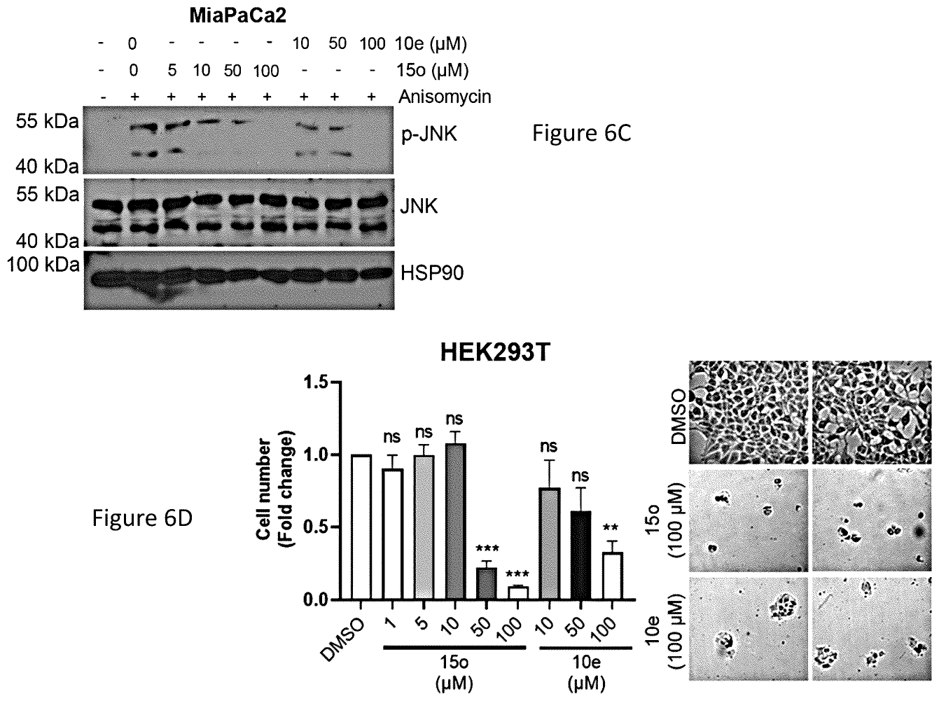

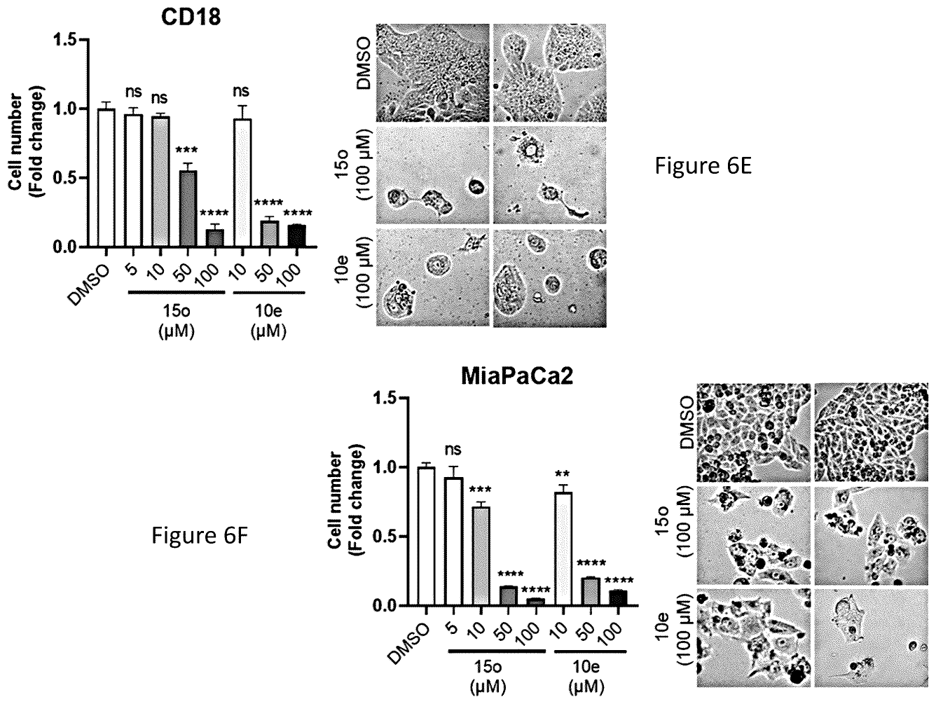

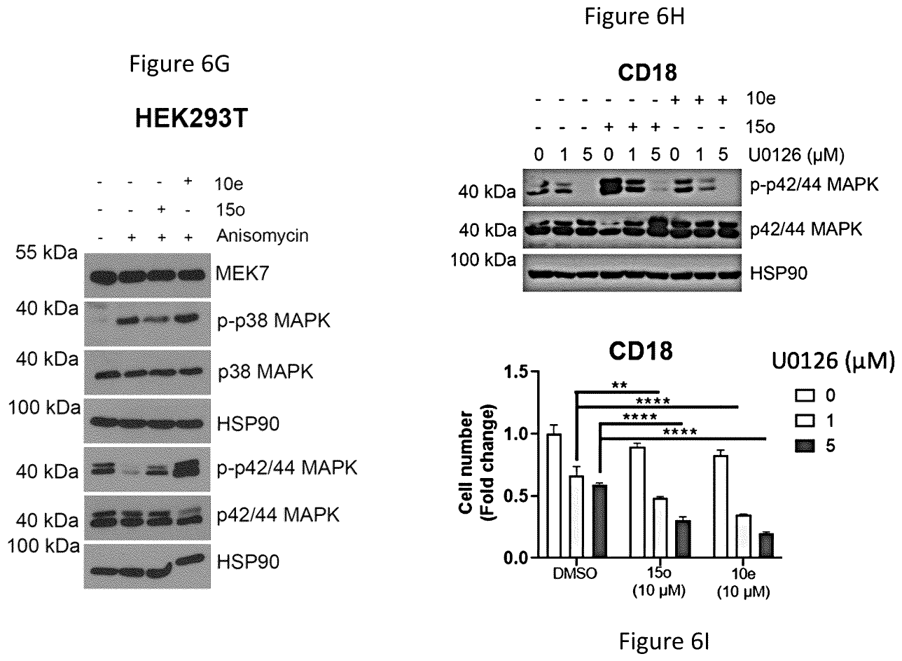

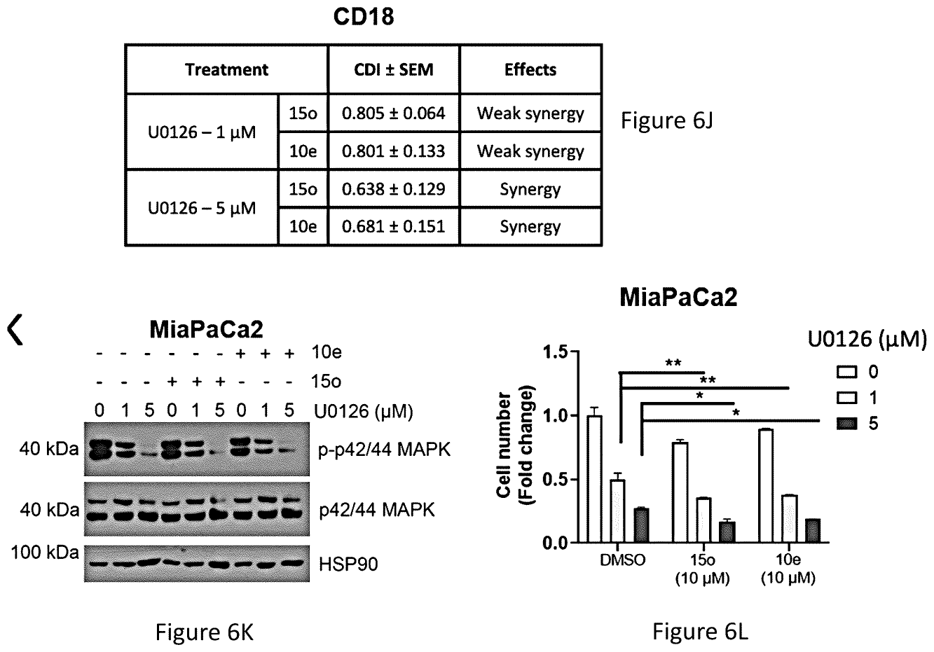

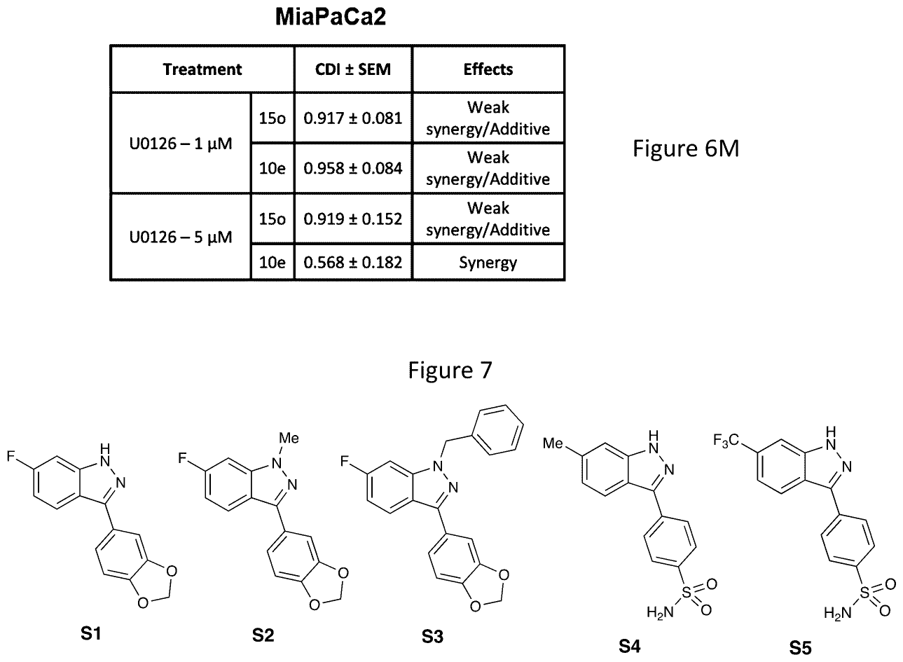

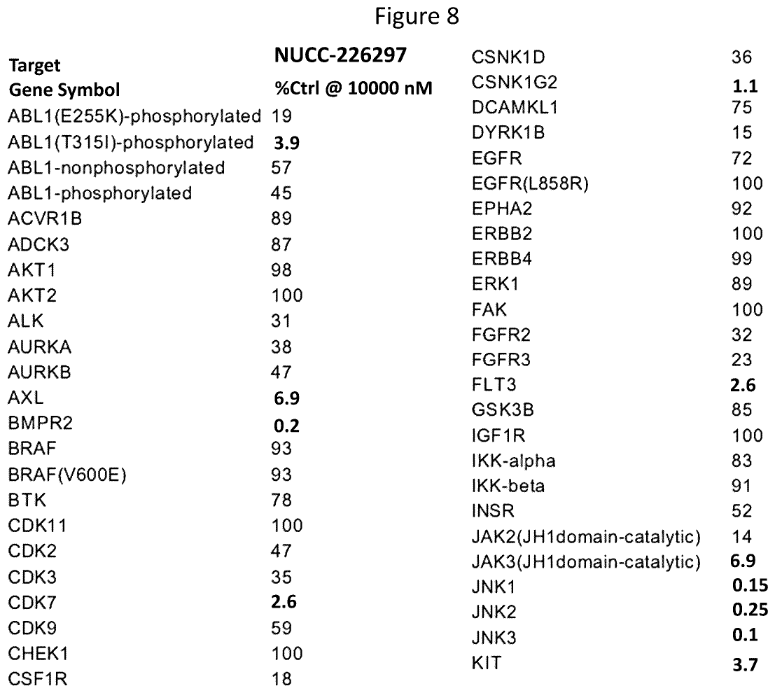

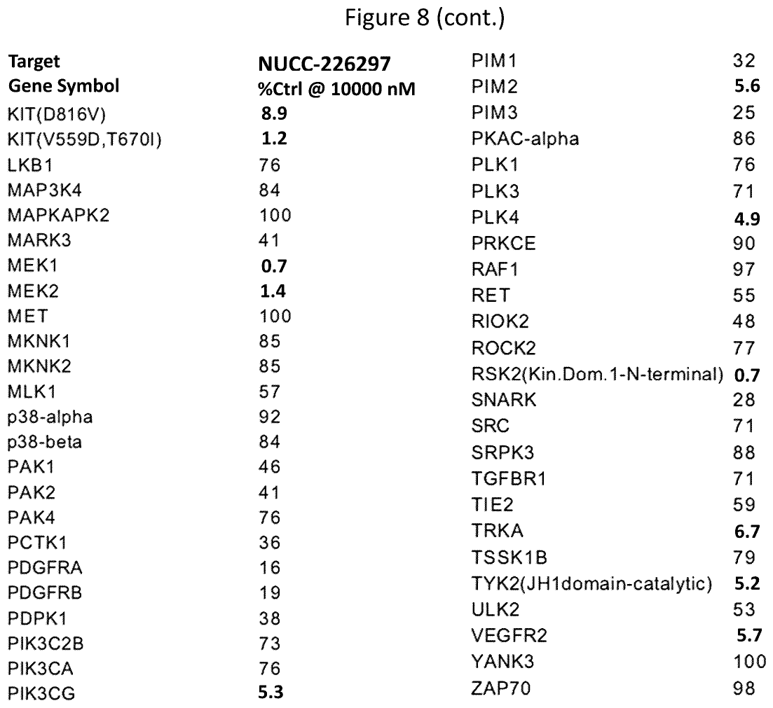

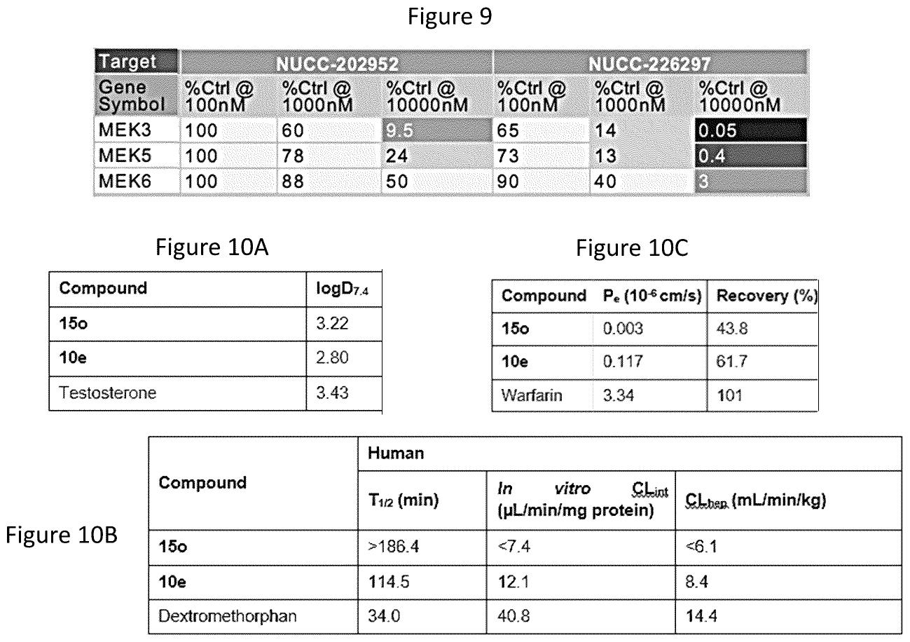

To evaluate the cellular efficacy of our two MEK4 inhibitors, we examined the effects of 15o and 10e on JNK phosphorylation. HEK293T cells were pre-treated with anisomycin, a potent JNK agonist²⁷⁻²⁸, before treatment with increasing doses of 15o and 10e. Treatment with 15o and 10e attenuated anisomycin-induced INK phosphorylation in a dose-dependent manner (FIG. 6A). Additionally, our compounds decreased. HEK293T cell proliferation in a dose-dependent manner (FIG. 6D). We also examined the effects of these inhibitors on JNK phosphorylation and proliferation of two pancreatic cancer cell lines (CD18 and MiaPaCa2). As with the HEK293T cells, our inhibitors attenuated anisomycin-induced JNK phosphorylation and decreased proliferation (FIGS. 6B, 6C, 6E, 6F).

We next evaluated the effects of our inhibitors on the phosphorylation activity and expression of MEK1/2, MEK3/6, and MEK7. HEK293T cells were pre-treated with anisomycin and then treated with either 15o or 10e. There was no effect on the expression of MEK7 (FIG. 6G). However, as shown previously 2728 , treatment with anisomycin did induce phosphorylation of the MEK3/6 effector p38 MAPK, which was not affected by either 15o or 10e (FIG. 6G). We also evaluated the effects on p42/44 MAPK (ERK1/2), which functions downstream of MEK1/2. Consistent with previously published reports²⁹⁻³¹, we observed that anisomycin decreases p42/44 MAPK phosphorylation in HEK293T cells (FIG. 6G). Notably, treatment with 15o and 10e reversed the effects of anisomycin on p42/44 MAPK phosphorylation. Though we could not discount other off target effects playing a role in decreased cellular proliferation, we found these results to be encouraging in that they revealed some degree of MEK family selectivity in a cellular system. Additionally, these results lay the groundwork for additional compound development, and we aim to develop tool probes that will be able to rule out off-target engagement in the future.

In additional experiments, we observed the effects of 15o and 10e on p42/44 MAPK phosphorylation CD18 and MiaPaCa2 cells. Treatment with 1.5o and 10e increased p42/44 MAPK phosphorylation in CD18 cells (FIG. 6H), which was suppressed by co-treating with the MEK1/2 inhibitor U0126. While 15o and 1.0e did not induce p42/44 MAPK phosphorylation in MiaPaCa2 cells, treatment with U0126 nonetheless decreased p42/44 MAPK phosphorylation (FIG. 6K). Finally, we evaluated the effects of combining 15o or 10e with U0126 on pancreatic cancer cell proliferation. The combination of U0126 and MEK4 inhibitors suppressed the proliferation of CD18 (FIGS. 6H, 6I) and MiaPaCa2 (FIGS. 6K, 6L) cell lines, with the combination more effective in CD18 cells (FIGS. 6J, 6M). Our findings underscore crosstalk between the MEK1/2 and the MEK4 signaling pathways. Notably, others have also shown this connection between these two MEK pathways: Bernards and coworkers showed that treatment with MEK1/2 inhibitors could activate the MEK4 signaling pathway, with co-targeting of these two pathways resulting in synergistic anti-tumor effects.³² Our results indicate that inhibiting the MEK4 pathway activates the MEK1/2 pathway, with the combination of the MEK1/2 and MEK4 inhibitors demonstrating anti-tumor effects in pancreatic cancer cells.

Cell-permeable, potent, and selective MEK4 inhibitors have been developed. Our lead optimization was guided by an iterative cycle of computational modeling, experimental synthesis, and in vitro evaluation of our designed compounds. Additionally, we evaluated the selectivity of our lead analogs across the MEK family as well as across the selected kinome, in addition to various pharmacokinetic properties. Future work will focus on improving the inhibitor selectivity for MEK4 over other kinases while retaining potency and cell permeability. Compounds 10e and 15o successfully inhibited the phosphorylation of INK and displayed antiproliferative properties against pancreatic cancer lines CD18 and MiaPaCa2. Finally, the promising data from the co-treatment with MEK1/2 inhibitors serves as a proof-of-concept for potential combination therapy using MEK1/2 and MEK4 inhibitors against pancreatic cancer, especially in tumors that exhibit activation of MEK1/2 following treatment with MEK4 inhibitors.

### Abbreviations

MAPK, mitogen-activated protein kinase; ERK, extracellular signal-regulated kinase; MEK/MAP2K, mitogen-activated protein kinase kinase; MAPK, mitogen-activated protein kinase; HEK293, human embryonic kidney 293 cells; Cys, cysteine; Lys, lysine; Glu, glutamate; Met, methionine; Ser, serine; ATP, adenosine triphosphate; ADP, adenosine diphosphate; IC₅₀, half-maximal inhibitory concentration; K_{d}, dissociation constant; TK, tyrosine kinases; TKL, tyrosine kinase-like kinases; STE, serine/threonine kinases; CDK, cyclic dependent kinase; CMGC, cyclin-dependent kinases, mitogen-activated protein kinases, glycogen synthase kinases and CDK-like kinases; BRAT, v-raf murine sarcoma viral oncogene homolog B1; RAF, Rapidly Accelerated Fibrosarcoma; JNK, c-Jun N-terminal kinase; Pgp, P-glycoprotein; CYP, cytochrome P450.

### REFERENCES

1. Zhang, W.; Liu, H. T., MAPK signal pathways in the regulation of cell proliferation in mammalian cells. Cell Res. 2002, 12, 9.
2. Raman, M.; Chen, W.; Cobb, M. H., Differential regulation and properties of MAPKs. Oncogene 2007, 26, 3100.
3. Chang, L.; Karin, M., Mammalian MAP kinase signalling cascades. Nature 2001, 410, 37.
4. Cargnello, M.; Roux, P. P., Activation and function of the MAPKs and their substrates, the MAPK-activated protein kinases. Microbiol. Mol. Biol. Rev. 2011, 75, 50.
5. Akinleye, A.; Furqan, M.; Mukhi, N.; Ravella, P.; Liu, D., MEK, and the inhibitors: from bench to bedside. J. Hematol. Oncol. 2013, 6, 27.
6. Cheng, Y.; Tian, H., Current Development Status of MEK Inhibitors. Molecules 2017, 22.
7. Wang, C.; Wang, H.; Zheng, C.; Liu, Z.; Gao, X.; Xu, F.; Niu, Y.; Zhang, I.; Xu, P., Research progress of MEK1/2 inhibitors and degraders in the treatment of cancer. Eur. J. Med. Chem. 2021, 218, 113386.
8. Kwong, A. J.; Scheidt, K. A., Non-‘classical’ MEKs: A review of MEK3-7 inhibitors. Bioorg. Med. Chem. Lett. 2020, 30, 127203.
9. Wang, X.; Destrument, A.; Tournier, C., Physiological roles of MKK4 and MKK7: insights from animal models, Biochem. Biophys. Acta Mol. Cell Res. 2007, 1773, 1349.
10. Dhillon, A. S.; Hagan, S.; Rath, O.; Kolch, W., MAP kinase signalling pathways in cancer. Oncogene 2007, 26, 3279.
11. Bozyczko-Coyne, D.; Saporito, M. S.: Hudkins, R. L., Targeting the JNK Pathway for Therapeutic Benefit in CNS Disease. Curr. Drug Targets CNS Neurol. Disord. 2002, 1, 31.
12. Chang, T.-S., Isolation, Bioactivity. and Production of ortho-Hydroxydaidzein and ortho-Hydroxygenistein. Int. J. Mol. Sci. 2014, 15.
13. Cong, C.; Kluwe, L.; Li, S.; Liu, X.; Liu, Y.; Liu, H.; Gui, W.; Liu, T.; Xu, L., Paeoniflorin inhibits tributyltin chloride-induced apoptosis in hypothalamic neurons via inhibition of MKK4-JNK signaling pathway. J. Ethnopharmacol. 2019, 237,
14. Ogura, M.; Kikuchi, H.; Shakespear, N.; Suzuki, T.; Yamaki, J.; Homma, M. K.; Oshima, Y.; Homma, Y., Prenylated quinolinecarboxylic acid derivative prevents neuronal cell death through inhibition of MKK4. Biochem. Pharmacol. 2019, 162, 109.
15. Kim, N.; Park, J.; Gadhe, C. G.; Cho, S. J.; Oh, Y.; Kim, D.; Song, K., A Protoherberine Derivative HWY336 Selectively Inhibits MKK4 and MKK7 in Mammalian Cells: The Importance of Activation Loop on Selectivity. PLOS ONE 2014, 9, 091037.
16. Deibler, K. K.; Schiltz, G. E.; Clutter, M, R.; Mishra, R, K.; Vagadia, P. P.; O'Connor, M.; George, M. D.; Gordon, R.; Fowler, G.; Bergan, R.; Scheidt, K. A., Synthesis and Biological Evaluation of 3-Arylindazoles as Selective MEK4 Inhibitors. ChemMedChem 2019, 14, 615.
17. Klövekorn, P.; Pfaffenrot, B.; Juchum, M.; Selig, R.; Albrecht, W.; Zender, L.; Laufer, S. A., From off-to on-target: New BRAF-inhibitor-template-derived compounds selectively targeting mitogen activated protein kinase kinase 4 (MKK4). Eur. J. Med Chem. 2021, 210, 112963.
18. Jiang, J.; Jiang, B.; He, Z.; Ficarro, S. B.; Che, J.; Marto, J. A.; Gao, Y.; Zhang, T.; Gray, N. S., Discovery of Covalent MKK4/7 Dual Inhibitor. Cell Chem. Biol. 2020, 27, 1553.
19. Deibler, K. K.; Mishra, R. K.; Clutter, M. R.; Antanasijevic, A.; Bergan, R.; Caffrey, M.; Scheidt, K. A., A Chemical Probe Strategy for Interrogating Inhibitor Selectivity Across the MEK Kinase Family. ACS Chem. Biol. 2017, 12, 1245.
20. Mishra, R. K.; Deibler, K. K.; Clutter, M. R.; Vagadia, P. P.; O'Connor, M.; Schiltz, G. E.; Bergan, R.; Scheidt, K. A., Modeling MEK4 Kinase Inhibitors through Perturbed Electrostatic Potential Charges, J. Chem. Inf. Model. 2019, 59, 4460.
21. Matsumoto, T.; Kinoshita, I.; Kirii, Y.; Tada, T.; Yamano, A., Crystal and solution structures disclose a putative transient state of mitogen-activated protein kinase kinase 4. Biochem. Biophys. Res. Common. 2012, 425, 195.
22. Matsumoto, T.; Kinoshita, T.; Kirii., Y.; Yokota, K,; Hamada, K.; Tada, T., Crystal structures of MKK4 kinase domain reveal that substrate peptide binds to an allosteric site and induces an auto-inhibition state. Biochem. Biophys. Res. Commun. 2010, 400, 369.
23. Sutanto, F.; Konstantinidou, M.; Dömling, A., Covalent inhibitors: a rational approach to drug discovery. RSC Med. Chem. 2020, 11, 876.
24. Zegzouti, H.; Zdanovskaia, M.; Hsiao, K; Goueli, S. A., ADP-Glo: A Bioluminescent and Homogeneous ADP Monitoring Assay for Kinases. Assay Drug Dev. Techn. 2009, 7, 560.
25. Tanega, C.; Shen, M.; Mott, B. T.; Thomas, C. J.; MacArthur, R.; Inglese, J.; Auld, D. S., Comparison of bioluminescent kinase assays using substrate depletion and product formation. Assay Drug Dev. Team. 2009, 7, 606.
26. Ballatore, C.; Huryn, D. M.; Smith Iii, A. B., Carboxylic Acid (Bio)Isosteres in Drug Design. ChemMedChem 2013, 8, 385.
27. Hazzalin, C. A.; Cano, E.; Cuenda, A.; Barratt, M. J.; Cohen, P.; Mahadevan, L. C., p38/RK is essential for stress-induced nuclear responses: JNK/SAPKs and c-Jun/ATF-2 phosphorylation are insufficient. Curr. Biol. 1996, 6, 1028.
28. Hazzalin, C. A.; Le Parise, R.; Cano, E.; Mahadevan, L. C., Anisomycin selectively desensitizes signalling components involved in stress kinase activation and fos and jun induction. Mol. Cell. Biol. 1998, 18, 1844.
29. Zhang, H.; Shi, X.; Hampong, M.; Blanis, L.; Pelech, S., Stress-induced Inhibition of ERK1 and ERK2 by Direct Interaction with p38 MAP Kinase. J. Biol. Chem. 2001, 276, 6905.
30. Croons, V.; Martinet, W.; Herman, A. G.; Timmermans, J.-P.; De Meyer, G. R. Y., The Protein Synthesis Inhibitor Anisomycin Induces Macrophage Apoptosis in Rabbit Atherosclerotic Plaques through p38 Mitogen-Activated Protein Kinase. J. Pharmacol. Exp. Ther. 2009, 329, 856.
31. Li, J.-y.; Huang, J.-y.; Li, M.; Zhang, H.; Xing, B.; Chen, G.; Wei, D.; Gu, P.-y.; Hu, W.-x., Anisomycin induces glioma cell death via down-regulation of PP2A catalytic subunit in vitro. Acta Pharmacol. Sin. 2012, 33, 935.
32. Xue, Z.; Vis, D. J.; Bruna, A.; Sustic, T.; van Wageningen, S.; Batra, A. S.; Rueda, O. M.; Bosdriesz, E.; Caldas, C.; Wessels, L. E. A.; Bernards, R., MAP3K1 and MAP2K4 mutations are associated with sensitivity to MEK inhibitors in multiple cancer models. Cell Res. 2018, 28, 719.

### EXPERIMENTAL SECTION

### General Information

All reactions were carried out in glassware with magnetic stirring. Purification of reaction products were carried out by flash chromatography on Biotage Isolera 4 systems with Ultra-grade silica cartridges. Analytical thin layer chromatography was performed on FM Reagent 0.25 mm silica gel 60-F plates. Visualization was accomplished with UV light. Monitoring of reactions via UPLC was done on a WATERS Acquity-H UPLC-MS with a single quad detector (SQD, ESI). ¹H NMR spectra were recorded on AVANCE III 500 MHz w/ direct cryoprobe (500 MHz) spectrometer and are reported in ppm using solvent as an internal standard (DMSO-d₆ at 2.50 ppm, MeOD at 3.31 ppm). Data are reported as (s=singlet; d=doublet, t=triplet, q=quartet, hept=heptet, m=multiplet, b=broad; coupling constant(s) in Hz; integration.) Proton-decoupled ¹³C NMR spectra were recorded on an AVANCE III 500 MHz w/ direct cryoprobe (126 MHz) spectrometer and are reported in ppm using solvent as an internal standard (DMSO-d6 at 39.5 ppm, MeOD at 49.0 ppm). Compounds were found to be>90% pure, assessed by ¹H NMR (AVANCE III 500 MHz w/ direct cryoprobe (500 MHz) spectrometer) and UPLC-MS (WATERS Acquity-H UPLC-MS with a SQD). High-resolution mass spectrometry (HRMS) was obtained using an Agilent 6201 LCMS-TOF (ESI), Agilent LCMS 6545 QTOF (ESI), or Bruker IMPACT II (ESI). All microwave-assisted reactions were carried out in a Biotage initiator.

### General Procedures

### ADP-Glo Kinase Activity Assay

Procedure for a 5 μL Kinase reaction using the ADP-Glo Kinase Assay Kit from Promega. Staurosporine was used as the positive control, and DMSO as the negative control. Activity of recombinant active full-length human MEK4 (Carna Biosciences) was measured using the ADP-Glo assay with recombinant full-length human p38α (MAPK14). Kinase reactions with MEK7 (Carna Biosciences) were done at the same concentration and amounts with recombinant full-length human p38α (MAPK14).

Using a Mantis, a mixture of MEK and p38α was dispensed into a 384 low volume white Proxiplate. For each well, 4 ng of MEK4 and 600 ng of p38α in Kinase buffer (CST) were used, and 1 μL of this mixture was dispensed per well.

Inhibitor was prepared in a DMSO solution. A dose response dilution was done in a 384-well Echo transfer plate. Using an Echo Access, 10 nL of each solution was dispensed into the same 384 low volume white Proxiplate as above. This mixture was allowed to incubate for 30 minutes at room temperature.

Using a Mantis, 1 μL, of an 8 μM ATP solution was added to each well, and the plate was allowed to incubate for another 1 hour at room temperature. Using the Mantis again, 4 μL of ADP Glo Reagent was dispensed into each well, and the plate was allowed to incubate for 45 min at room temperature. Finally, 9 μL of Kinase Detection Reagent was added manually to each well, and the plate was allowed to incubate for 45 minutes at room temperature. Luminescence was read on a Synergy Neo2 Plate Reader with filter cubes 3 and 114. Integration time was 0.20 seconds, gain set to 160, read height set at 6 mm. All experiments were done in triplicate on the plate, and at least in one separate technical duplicate. Each experiment's IC₅₀ values are reported as (IC₅₀±SEM) and can be found under each compound's characterization data in the tabulated data section.

### Docking Protocols

The refined MEK4 PDB was used for docking.¹ After preparing the protein and the ligand set, all small-molecule structures were docked into the active site of MEK4 using Schrodinger Maestro software. We generated a 10 Å×10 Å×10 Å cube in the ATP binding site of the MEK4 structure and used all the default parameters for docking.

### Cell Culture

Human pancreatic cancer cell lines (CD18/HPAF-II and MiaPaCa2) and HEK293T cells were obtained from American Type Culture Collection. All cell lines were cultured according to the manufacturer's protocol. All media contained 10% Fetal Bovine Serum (FBS) and antibiotics (100 U/mL Penicillin and 100 μg/mL Streptomycin).

### Chemicals for Cell Culture

### Immunoblotting

Whole cell extracts of cultured cells were prepared in radioimmunoprecipitation (RIPA) lysis buffer supplemented with phosphatase and protease inhibitors (Calbiochem). Protein concentration was determined by the bicinchoninic acid assay (BCA) (ThermoFisher) and separated by the SDS-PAGE technique. The following antibodies were obtained from Cell Signaling and used at the dilution recommended by the manufacturer: phospho-JNK (#4668), total JNK (#9252), MEK7 (#4172), phospho-ERK1/2 (phospho-p42/44 MAPK, #9101), phospho-p38 MAPK (#9211), and total p38 MAPK (#9212). Total ERK1/2 (p42/44 MAPK, sc-514302) and HSP90 (sc-7940) were obtained from Santa Cruz Biotechnology and used at the dilution recommended by the manufacturer. Secondary anti-mouse IgG (A4416) and anti-rabbit IgG (A6667) antibodies were purchased from Sigma-Aldrich and used a 1:4000 dilution. Images of blots were acquired on HyBlot ES Autoradiography Film (Thomas Scientific, Swedesboro, NJ, USA) following incubation with SuperSignal West Pico PLUS (ThermoFisher).

### Synthesis of Indazole Precursor S6

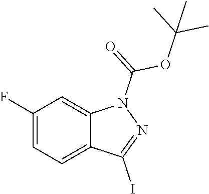 [SMILES: CC(C)(C)OC(=O)n1nc(I)c2ccc(F)cc21]

tert-butyl 6-fluoro-3-iodo-1H-indazole-1-carboxylate. (S6) To a solution of 6-fluoro-1H-indazole (10.00 g, 73.5 mmol) in DMF (147 mL) was added KOH (9.07g, 162 mmol), followed by I₂ (24.2 g, 95.5 mmol). The reaction was monitored by TLC or UPLC and allowed to run to completion. To this solution, di-tert-butyl dicarbonate (19.2 g, 88.2 mmol) was added, and the reaction was again monitored by TLC/UPLC and allowed to run to completion. Reaction was poured in an ice bath with saturated aqueous Na₂S₂O₃, adding more saturated aqueous Na₂S₂O₃ until all iodine color disappeared. The mixture was then transferred to a fritted funnel and washed with water and hexanes to afford S6 (22.2 g, 84%) as a pale-yellow solid. Analytical data for S6: ¹H NMR (500 MHz, DMSO-d₆) δ 7.75 (dd, J=9.6, 2.3 Hz, 1H), 7.59 (dd, J=8.8, 5.1 Hz, 1H), 7.33 (td, J=9.0. 2.3 Hz, 1H), 1.64 (s, 9H). ¹³C NMR (126 MHz, DMSO-d₆) δ 163.4 (d, J_{C-F}=246.7 Hz), 147.4, 139.3 (d, J_{C-F}=13.7 Hz), 126.6. 124.0 (d, J_{C-F}=11.3 Hz), 113.6 (d, J_{C-F}=25.8 Hz), 100.6 (d, J_{C-F}=28.9 Hz), 85.5, 27.6. HRMS (electrospray): Exact mass calculated for C₁₂H₁₂FIN₂O₂Na⁺ [M+Na], 384.9825. Found 384.9812.

### General Procedure (1) for Suzuki Couplings:

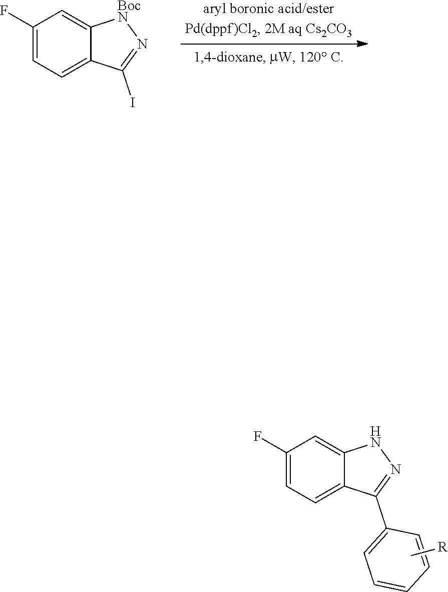

To a flame-dried microwave vial, S6 (1.2 equiv.), PdCl₂(dppf) (0.05 equiv.), and aryl boronic acid ester (1 equiv.) was added. The vial was crimped and purged with nitrogen. To this vial was added degassed 1,4-dioxane (0.40 M) and 2M aqueous Na₂CO₃ (0.40 M). This mixture was microwaved at 120° C. for 1 hour. The solution was diluted with water and extracted with EtOAc (3×10 mL). The organic layer was separated, dried over Na₂SO₄, and then concentrated in vacuo. The residue was purified by flash column chromatography (0-80% EtOAc/Hex over 20 column volumes) to afford the Suzuki coupling product.

### General Procedure (2) for Borylations:

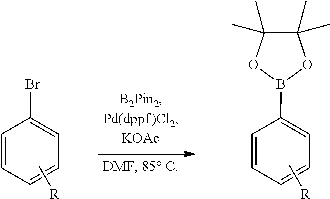

To a flame-dried round bottom was added the aryl bromide (1 equiv.), 4,4,4,4′,5,5,5′,5′-octamethyl-2,2′-bi(1,3,2-dioxaborolane) (1.2 equiv.), Pd(dppf)Cl₂ (applicable for both para- and meta-substituted compounds) or Pd(PPh₃)Cl₂ (better yielding for para-substituted) (0.05 equiv.), and KOAc (2.0 equiv). Degassed, anhydrous DMF (0.2 M) was added. The reaction was heated at 85° C. and monitored by UPLC. After running overnight, the reaction was diluted in water and extracted with EtOAc (3×30 mL). The organic layer was heavily washed with brine, dried over Na₂SO₄ and concentrated in vacuo. The residue was purified by flash column chromatography (SiO₂, EtOAc/Hex) to afford product.

### General Procedure (3) for Sulfonylations:

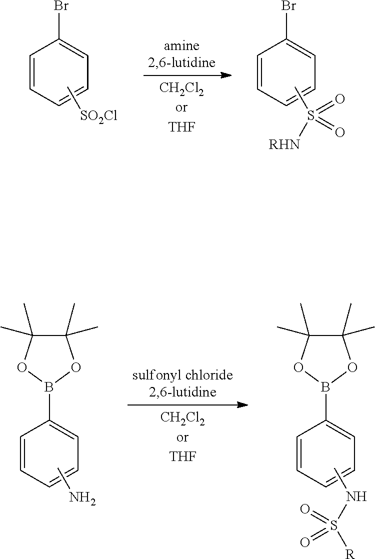

To a flame-dried round bottom was added the amine (1 equiv.), anhydrous CH₂Cl₂ or THF (0.25 M), and 2,6-lutidine (2.0 equiv.). Lutidine prevented formation of side products, compared to triethylamine and pyridine. Some products were successfully isolated or carried forward after employing pyridine or triethylamine in THF al the same equivalence. Reactions were dropped to 0° C. and sulfonyl chloride (1.1 equiv.) added dropwise (dissolved in a small amount of CH₂Cl₂ if solid). The reaction was monitored by UPLC. After running overnight, the reaction was diluted in water and extracted with CH₂Cl₂ (EtOAc if reaction was in THF). The organic layer was dried over Na₂SO₄ or dried via Biotage phase separator and concentrated in vacuo. The residue was purified by , flash column Chromatography (SiO₂, EtOAc/Hex) to afford product. Reactions yielded product in 50-85% yield.

### Procedure A: General Synthesis of Compounds 8b-f

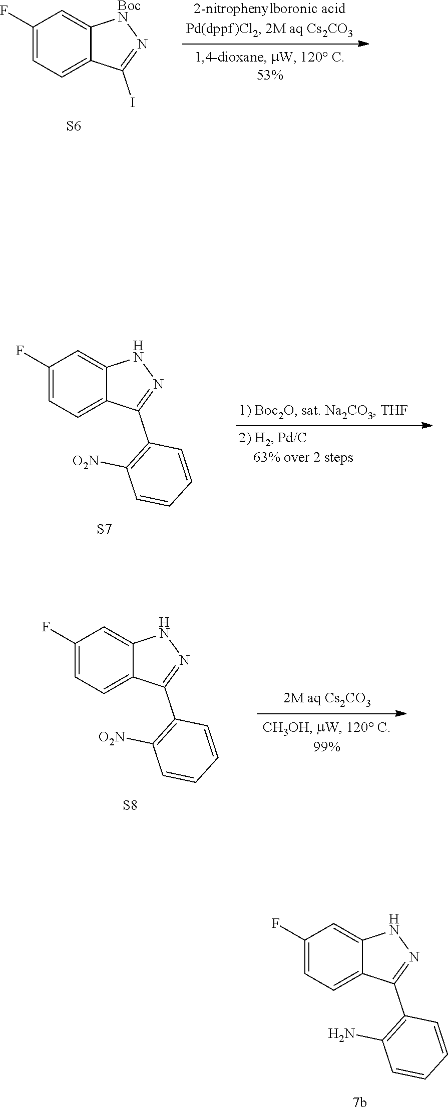

### Synthesis of S7. Prepared According to General Procedure 1.

Analytical data: ¹H NMR (500 MHz, Methanol-d₄) δ 8.03 (d, J=8.1 Hz, 1H), 7.82 (dd, J=7.3, 1.6 Hz, 2H), 7.70 (ddd, J=8.5, 6.6, 2.3 Hz, 1H), 7.59 (dd, J=5.0 Hz, 1H), 7.28 (dd, J=9.3, 2.1 Hz, 1H), 7.02 (td, J=9.1, 2.2 Hz, 1H).

Synthesis of Cert-butyl 3-(2-aminophenyl)-6-fluoro-1H-indazole-1-carboxylate S8. To a solution of 6-fluoro-3-(2-nitrophenyl)-1H-indazole S7 (1.68 g, 6.53 mmol) in THF (32.7 mL), di-tert-butyl decarbonate (2.85 g, 13.1 mmol) and saturated aqueous Na₂CO₃ (32.7 mL) were added. The reaction was stirred vigorously overnight. The solution was diluted with brine and extracted with EtOAc (3×25 mL). The organic layer was separated, dried over Na₂SO₄, then concentrated in vacuo. Tert-butyl 6-fluoro-3-(2-nitrophenyl)-1H-indazole-1-carboxylate residue was taken directly to the next step. Boc protection was done to make purification of the following product easier.

Analytical data of intermediate: 1 H NMR (500 MHz, Methanol-d₄) δ 8.04 (dd, J=8.9. 5.1 Hz, 1H), 7.92 (dd, J=9.6, 2.3 Hz, 1H), 7.72 (dd, J=7.8, 1.6 Hz, 1H), 7.28-7.20 (m, 2H), 6.95 (dd, J=8.2, 1.1 Hz, 1H), 6.85 (td, J=7.5, 1.2 Hz, 1H), 1.76 (s, 9H).

To a solution of text-butyl 6-fluoro-3-(2-nitrophenyl)-1H-indazole-1-carboxylate residue in degassed methanol (9.4 mL), H₂/C (3.79 mg, 1.88 mmol) was added, and the solution purged with argon. The solution was then purged with H₂. An additional input of H₂ was left in the headspace for a constant 1 atm pressure of H₂. The reaction was stirred overnight (product should start crashing out soon after hydrogenation starts). The solution was filtered through celite, rinsing with EtOAc (Product is not very soluble in methanol). This was concentrated in vacuo. The residual solid was purified by flash column chromatography (SiO₂, 0-20% EtOAc/Hex) to afford tort-butyl 3-(2-aminophenyl)-6-fluoro-1H-indazole-1-carboxylate (1.35g, 63% over 2 steps) as a white solid.

Analytical data of S8: ¹H NMR (500 MHz, DMSO-d₆) δ 8.09 (dd, J=8.9, 5.2 Hz, 1H), 7.85 (dd, J=9.8, 2.4 Hz, 1H), 7.69 (dd, J=7.8, 1.6 Hz, 1H), 7.33 (td, J=9.0, 2.4 Hz, 1H), 7.20 (ddd, J=8.5, 7.1, 1.6 Hz, 1H), 6.88 (dd, J=8.2, 1.2 Hz, 1H), 6.73 (td, J=7.4, 1.2 Hz, 1H), 6.17 (s, 2H). 1.67 (s, 9H), ¹³C NMR (126 MHz, DMSO-d₆) 162.6 (d, J_{C-F}=245.8 Hz), 148.5, 148.2, 147.0, 140.2 (d, J_{C-F}=13.4 Hz), 130.2, 129.6, 124.3 (d, J_{C-F}11.0 Hz), 121.2, 115.9 (d, J_{C-F}=20.0 Hz), 113.1, 112.9, 112.8, 100.7 (d, J_{C-F}=28.3 Hz), 85.0, 27.6.

Synthesis of 7b. To a flame-dried microwave vial, tart-butyl 3-(2-aminophenyl)-6-fluoro-1H-indazole-1-carboxylate (200 mg, 0.61 mmol), methanol (6.1 mL) and 2M aqueous Na₂CO₃ (2.0 mL) were added. The vial was crimped, and this mixture was microwaved at 120° C. for 15 minutes. The solution was extracted with EtOAc (3×10 mL). The organic layer was separated, dried over Na₂SO₄, then concentrated in vacuo to afford 7b (138 mg, 99%) as a white solid.

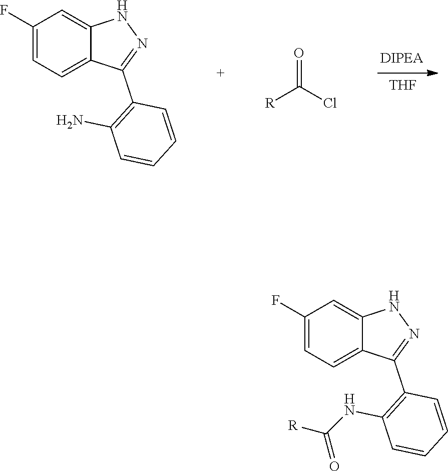

In a flame-dried vial, a solution of 7b (1 equiv.) in THF (0.20 M) was cooled to 0° C. DIPEA (1.2 equiv) was added dropwise. After stirring for a few minutes, acyl chloride (1.1 equiv.) was added dropwise. The reaction was allowed to reach room temperature and monitored by TLC (if reaction is run for too long, dialkylated product will be isolated). The reaction was concentrated in vacuo, and the residue was purified by flash column chromatography. Reaction did not work with protected indazole.

### Tabulated Data of Compounds 8a-f

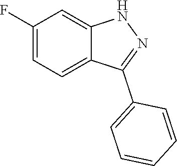 [SMILES: Fc1ccc2c(-c3ccccc3)n[nH]c2c1]

6-fluoro-3-phenyl-1H-indazole (8a). Prepared according to General Procedure 1 from indazole S6 (300 mg, 0.828 mmol), residue purified by flash column chromatography (SiO₂, 0-10% EtOAC/Hex) to afford 6-fluoro-3-phenyl-1H-indazole (134 mg, 76%) as an off-white solid. Analytical data: ¹H NMR (500 MHz, DMSO-d₆) δ 13.29 (s, 1H), 8.08 (dd, J=8.9, 5.1 Hz, 1H), 7.99-7.94 (m, 2H), 7.52 (dd, J=8.4, 6.9 Hz, 2H), 7.44-7.39 (m, 1H), 7.37 (dd, J=9.5, 2.3 Hz, 1H), 7.08 (td, J=9.2, 2.3 Hz, 1H). ¹³C NMR (126 MHz, DMSO-d₆) δ 161.2 (d, J_{C-F}=241.9 Hz), 143.5, 141.7 (d, J_{C-F}=12.6 Hz), 133.2, 128.9, 127.9, 126.8, 122.6 (d, J_{C-F}=10.9 Hz), 117.3, 110.6 (d, J_{C-F}=25.8 Hz), 95.9 (d, J_{C-F}=25.8 Hz). HRMS (ESI): Exact mass calculated for C₁₃H₁₀FN₂⁺ [M+H], 213.0828. Found 213.0826. IC₅₀: 0.31 μM [0.22±0.11, n=3; 0.40±0.18, n=3]

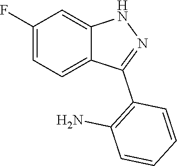 [SMILES: Nc1ccccc1-c1n[nH]c2cc(F)ccc12]

2-(6-fluoro-1H-indazol-3-yl)aniline (8b). Prepared according to General Procedure A. See details above. Analytical data: ¹H NMR (500 MHz, DMSO-d₆) δ 13.24 (s, 1H), 7.99 (dd, J=9.0, 5.2 Hz, 1H), 7.66 (dd, J=7.7, 1.6 Hz, 1H), 7.37 (dd, J=9.5, 2.3 Hz, 1H), 7.11 (ddd, J=8.5. 7.2, 1.6 Hz, 1H), 7.06 (td, J=9.1, 2.3 Hz, 1H), 6.85 (dd, J=8.1, 1.2 Hz, 1H), 6.71 (td, J=7.4, 1.2 Hz, 1H), 6.05 (s, 2H). ¹³C NMR (126 MHz, DMSO-d₆) δ 161.3 (d, J_{C-F}=242.0 Hz), 146.2, 143.8, 141.0 (d, J=12.9 Hz), 128.9, 128.5, 123.2 (d, J_{C-F}=11.1 Hz), 118.0, 115.7 (d, J_{C-F}=19.5 Hz), 115.4, 110.3, 110.1, 95.7 (d, J_{C-F}=25.8 Hz). HRMS (ESI): Exact mass calculated for C₁₃H₁₀FN₃⁺ [M+H], 228.0937. Found 228.0931. IC₅₀>80 μM

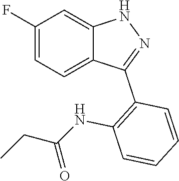 [SMILES: CCC(=O)Nc1ccccc1-c1n[nH]c2cc(F)ccc12]

N-(2-(6-fluoro-1H-indazol-3-yl)phenyl)propionamide (8c). Prepared according to General Procedure A from Sb to afford product (50 mg, 80%) as a white solid. Analytical data: ¹H NMR (500 MHz, DMSO-d₆) δ 13.54 (s, 1H), 10.34 (s, 1H), 8.24 (d, J=8.2 Hz, 1H), 7.86 (dd, J=9.0, 5.1 Hz, 1H), 7.80 (dd, 7.7, 1.6 Hz, 1H), 7.45 -7.38 (m, 2H), 7.27 (td, J=7.5, 1.3 Hz, 1H), 7.09 (td, J=9.2, 2.3 Hz, 1H), 2.28 (q, J=7.5 Hz, 2H), 1.06 (t, J=7.6 Hz, 3H). ¹³C NMR (126 MHz, DMSO-d₆) 171.75, 161.48 (d, J_{C-F}=242.5 Hz), 142.59, 141.20 (d, J_{C-F}=13.0 Hz), 136.04, 129.48, 128.42, 123.85, 122.79 (d, J_{C-F}=11.1 Hz), 122.45, 122.39, 118.03, 110.76 (d, J_{C-F}=26.1 Hz), 95.97 (d, J_{C-F}=25.9 Hz), 30.05, 9.49. HRMS (electrospray): Exact mass calculated for C₁₆H₁₅FN₃O⁺ [M+H], 284.1199. Found 284.1194. IC₅₀: >80 μM

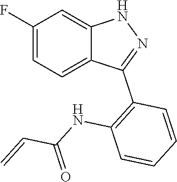 [SMILES: C=CC(=O)Nc1ccccc1-c1n[nH]c2cc(F)ccc12]

N-(2-(6-fluoro-1H-indazol-3-yl)phenyl)acrylamide (8d). Prepared according to General Procedure A with the corresponding acid chloride from 8b to afford 8d (50 mg, 58%) as a white solid. Analytical data: ¹H NMR (500 MHz, DMSO-d₆) δ 13.54 (bs, 1H), 10.52 (s, 1H), 8.27 (d, J=8.2 Hz, 1H), 7.88-7.79 (m, 2H), 7.48-7.40 (m, 2H), 7.32 (td, J=7.5, 1.3 Hz, 1H), 7.08 (td, J=9.2, 2.3 Hz, 1H), 6.32 (dd, J=17.1, 10.1 Hz, 1H), 6.23 (dd, J=17.0, 1.9 Hz, 1H), 5.75 (dd, J=10.0, 1.8 Hz, 1H). ¹³C NMR (126 MHz, DMSO-d₆) δ 163.3, 161.5 (d, J_{C-F}=242.7 Hz), 142.4, 141.2 (d, J_{C-F}=13.0 Hz), 135.7, 132.3, 129.7, 128.5, 126.7, 124.4, 123.1, 122.8, 122.7 (d, J_{C-F}=11.1 Hz), 118.1, 110.8 (d, J_{C-F}=26.2 Hz), 96.0 (d, J_{C-F}=25.9 Hz). HRMS (ESI): Exact mass calculated for C₁₆H₁₃FN₃O⁺ [M+H], 282.1042. Found 282.1037. IC₅₀: >80 μM

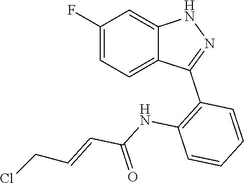 [SMILES: O=C(/C=C/CCl)Nc1ccccc1-c1n[nH]c2cc(F)ccc12]

N-(2-(6-fluoro-1H-indazol-3-yl)phenyl)propionamide (8e). Prepared according to General Procedure A with the corresponding acid chloride from 8b to afford product (86 mg, 46%) as a pale yellow solid. Acid chloride was made as follows: A flame dried round-bottom flask containing phenyl hypochloroselenoite (445 mg, 2.323 mmol) was charged with acetonitrile (134 mL). The reaction mixture was stirred for 5 minutes at room temperature to give a homogenous red solution. But-3-enoic acid (2.00 g, 134 mL, 23.2 mmol) was added by pipette (solution turned pale yellow at this point). N-chlorosuccinimide (NCS, 3.41 g, 25.6 mmol) in acetonitrile (134 mL) was then added by addition funnel. Reaction was then concentrated in sumo, and the resulting residue was resuspended in diethyl ether. The ether layer was washed with water (2×150 mL) and brine, dried over sodium sulfate, filtered, and concentrated. The acid was obtained (2.4 g, 86%). (E)-4-chlorobut-2-enoic acid (50 mg, 0.41 mmol) was azeotropically washed in toluene (3×). This was dissolved in THF, put under nitrogen, and cooled to 0° C. Oxalyl chloride (44 uL, 0.50 mmol) was carefully added, followed by DMF (75 uL, 0.96 mmol). The solution was stirred under nitrogen and the ice bath was pulled down. The reaction was run for about 30 minutes. This was concentrated in vacuo and put under high vacuum. The residue was used the same day without further purification or isolation. Analytical data: ¹H NMR (500 MHz, DMSO-d₆) δ 13.51 (s, 1H), 10.41 (s, 1H), 8.21 (d, J=8.2 Hz, 1H), 7.85-7.74 (m, 2H), 7.45 (ddd, J=8.4, 7.4, 1.6 Hz, 1H), 7.41 (dd, J=9.4, 2.3 Hz, 1H), 7.31 (td, J=7.5, 1.3 Hz, 1H), 7.06 (td, J=9.2, 2.3 Hz 1H), 6.82 (dt, J=15.0, 6.1 Hz, 1H), 6.30 (dt, J=15.0, 1.6 Hz, 1H), 4.40 (dd, J=6.1, 1.5 Hz, 2H). ¹³C NMR (126 MHz, DMSO-d₆) δ 162.7, 161.4 (d, J_{C-F}=242.4 Hz), 142.4, 141.2 (d, J_{C-F}=13.1 Hz), 138.5, 135.6, 129.8, 128.5, 127.2, 124.6, 123.5, 123.1, 122.6 (d, J_{C-F}=11.0 Hz), 118.1, 110.7 (d, J=26.1 Hz), 96.0 (d, J_{C-F}=25.8 Hz), 43.3. HRMS (ESI): Exact mass calculated for C₁₇H₁₄ClFN₃O⁺ [M+H], 330.0809. Found 330.0799, IC₅₀: >80 μM

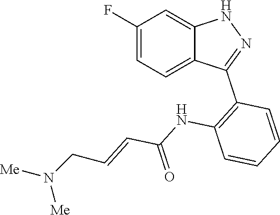 [SMILES: CN(C)C/C=C/C(=O)Nc1ccccc1-c1n[nH]c2cc(F)ccc12]

N-(2-(6-(fluoro-1H-indazol-3-yl)phenyl)propionamide (8f). Prepared according to General Procedure A, using 8e as the starting material. To a flame-dried vial was added a solution of 8f (50 mg, 0.15 mmol) in THF (0.51 mL). Dimethylamine (80 uL, 1.20 mmol) was then added dropwise. The reaction was heated to 50° C. and monitored by UPLC. The reaction was concentrated in vacuo and placed under high vacuum overnight. The residue was purified by flash column chromatography (SiO₂, 0-5% MeOH/DCM) to afford 11 (35 mg, 68%) as a white solid. Analytical data: ¹H NMR (500 MHz, DMSO-d₆) δ 13.54 (s, 1H), 10.42 (s, 1H), 8.28 (d, J=8.2 Hz, 1H), 7.86-7.79 (m, 2H), 7.47-7.39 (m, 2H), 7.30 (td, J=7.5, 1.3 Hz, 1H), 7.07 (td, J=9.1, 2.3 Hz, 1H), 6.71 (dt, J=15.5, 6.0 Hz, 1H), 6.13 (dt, J=15.5, 1.6 Hz, 1H), 3.02 (dd, J=5.9, 1.6 Hz, 2H), 2.15 (s, 6H). ¹³C NMR (126 MHz, DMSO-₆) 163.2, 161.5 (d, J_{C-F}=242.6 Hz), 142.5, 141.4, 141.2 (d, J_{C-F}=13.1 Hz), 135.9, 129.6, 128.4, 126.2, 124.2, 123.0, 122.8, 122.7, 118.1, 110.7 (d, J_{C-F}=26.0 Hz), 96.0 (d, J_{C-F}=25.5 Hz), 59.7, 45.1. HRMS (ESI): Exact mass calculated for C₁₉H₂₀FN₄O⁺ [M+H], 339.1621. Found 339.1618. IC₅₀: >80 μM

### Procedure B: General Synthesis of Compounds 9a-d

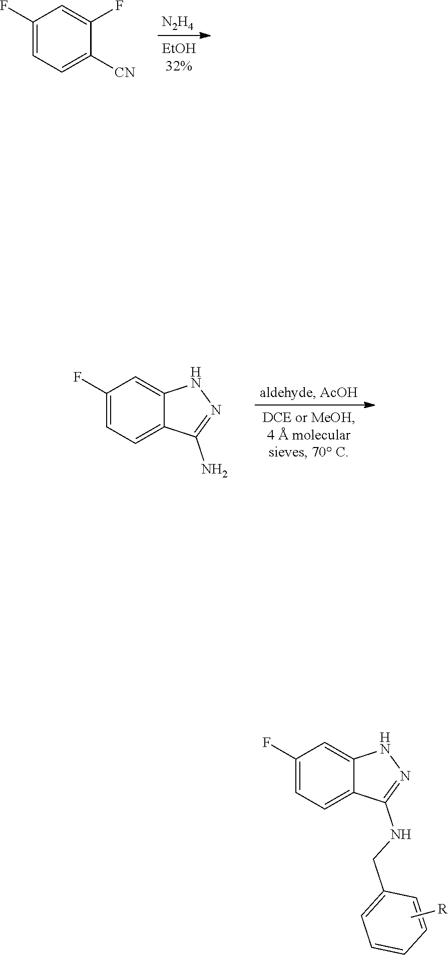

To a round bottom flask was added 2,4-fluorobenzonitrile (4.00 g, 28.8 mmol) and ethanol (140 mL). Hydrazine monohydrate (15.4 mL, 316 mmol) was then added slowly. The reaction was heated to 75° C. (behind a blast shield) overnight. The reaction was concentrated in vacuo, taken up in water and extracted with EtOAc (3×30 mL). The organic layer was washed with brine, dried over Na₂SO₄ and concentrated in vacuo to yield a solid crude product. The solid was purified by flash column chromatography (SiO₂, 30% then 40% EtOAc/Hex stepwise) to afford 6-fluoro-1H-indazol-3-amine (1.38 g, 32%) as an off-white solid. Spectra matches previously published data.² Analytical data for 6-fluoro-1H-indazol-3-amine: ¹H NMR (500 MHz, DMSO-d₆) δ 11.43 (s, 1H), 7.69 (dd, J=8.8, 5.4 Hz, 1H), 6.96 (dd, J=10.1, 2.2 Hz, 1H), 6.75 (td, J=9.1, 2.2 Hz, 1H), 5.40 (s, 2H). ¹³C NMR (126 MHz, DMSO-d₆) δ 161.8 (d, J==240.0 Hz), 149.2, 141.5 (d, J=13.0 Hz), 122.0 (d, J=11.5 Hz), 111.3, 106.6 (d, J=26.0 Hz), 94.7 (d, J=25.9 Hz).

To a flame-dried vial was added 6-fluoro-1H-indazol-3-amine (1 equiv.) and 4-fluoro-2-hydroxybenzaldehyde (1.1 equiv.) dissolved in dichloroethane (DCE) or methanol (0.1 M) with 4 Å molecular sieves, To this was added glacial acetic acid (3 equiv.). The reaction was heated to 70° C. and allowed to stir for an hour. Sodium triacetoxyborohydride (7 equiv.) was added, and the mixture was allowed to stir overnight at 50° C. Upon completion, the reaction was filtered, washed with saturated NaHCO₃, the aqueous layer backextracted with EtOAc (3×10 mL) and the organic layer concentrated in vacuo. The residue was purified by flash column chromatography to afford product.

### Tabulated Data of Compounds 9a-d

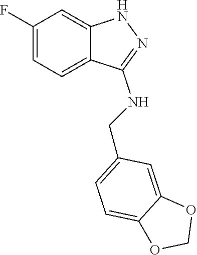 [SMILES: Fc1ccc2c(NCc3ccc4c(c3)OCO4)n[nH]c2c1]

N-(benzo[d][1,3]dioxol-5-ylmethyl)-6-fluoro-1H-indazol-3-amine (9a). General Procedure B was used with the corresponding aldehyde to afford 9a (33 mg, 35%) as a white solid. Analytical data: ¹H NMR (500 MHz, DMSO-₆) δ 11.47 (s, 1H), 7.76 (dd, J=8.8, 5.4 Hz, 1H), 6.98 (dd, J=10.1, 2.0 Hz, 2H), 6.89-6.82 (m, 2H), 6.78-6.73 (m, 1H), 6.53 (t, J=6.1 Hz, 1H), 5.96 (s, 2H), 4.35 (d, J6.1 Hz, 2H), ¹³C NMR (126 MHz, DMSO d₆) δ 161.9 (d, J_{C-F}=240.2 Hz), 149.7, 147.1, 145.8, 141.9 (d, J_{C-F}=12.9 Hz), 134.7, 121.8 (d, J_{C-F}=11.5 Hz), 120.6, 110.9, 108.1, 107.8, 106.5 (d, J_{C-F}=25.9 Hz), 100.7, 94.7 (d, J_{C-F}=25.9 Hz), 46.4. HRMS (ESI): Exact mass calculated for C₁₅H₁₃FN₃O₂⁺ [M+H], 286.0992. Found 286.0983. IC₅₀: 11 μM [9.4±1.5, n=3; 12±2.6, n=3]

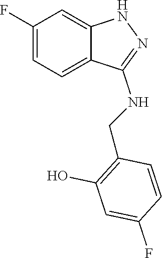 [SMILES: Oc1cc(F)ccc1CNc1n[nH]c2cc(F)ccc12]

5-fluoro-2-(((6-fluoro-1H-indazol-3-yl)amino)methyl)phenol (9b). General Procedure B was used with the corresponding aldehyde to afford 9b (22 mg, 24%) as a pale yellow solid. Analytical data: ¹H NMR (500 MHz, DMSO-d₆) δ 11.53 (s, 1H), 10.28 (s, 1H), 7.78 (dd, J=8.8, 5.4 Hz, 1H), 7.26 (dd, J=8.4, 7.1 Hz, 1H), 6.98 (dd, J=10.1, 2.2 Hz, 1H), 6.76 (td, J=9.1, 2.2 Hz, 1H), 6.66-6.49 (m, 3H), 4.34 (d, J=5.9 Hz, 2H). ¹³C NMR (126 MHz, DMSO-d₆) δ 162.0 (d, J_{C-F}=240.0 Hz), 161.6 (d, J_{C-F}=241.8 Hz), 156.4, 149.9, 142.0 (d, J_{C-F}=13.0 Hz), 130.1 (d, J_{C-F}=10.2 Hz), 123.0 (d, J_{C-F}=2.7 Hz), 122.0 (d, J_{C-F}=11.5 Hz), 110.7, 106.7 (d, J_{C-F}=25.9 Hz), 105.1 (d, J_{C-F}=20.9 Hz), 102.4 (d, J_{C-F}=23.4 Hz), 94.8 (d, J_{C-F}=26.0 Hz), 41.2. HRMS (ESI): Exact mass calculated for C₁₄H₁₂F2N₃O⁺ [M+H], 276.0948, Found 276.0948. IC₅₀: 11 μM [10±2.7, n=3; 11±2.8, n=3]

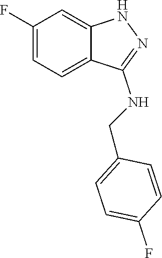 [SMILES: Fc1ccc(CNc2n[nH]c3cc(F)ccc23)cc1]

5-fluoro-N-(3-fluorobenzyl)-1H-indazol-3-amine (9c). General Procedure B was used with the corresponding aldehyde to afford 9c (30 mg, 35%) as a brown solid.

Analytical data: NMR (500 MHz, Methanol-d₄) δ 7.68 (dd, J=8.8, 5.2 Hz, 1H), 7.45-7.36 (m, 2H), 7.05-6,98 (m, 2H), 6.92 (dd, J=9.8, 2.2 Hz, 1H), 6.73 (td, J=9.1, 2.2 Hz, 1H), 4.49 (s, 2H). ¹³C NMR (126 MHz, Methanol-d₄) δ 164.6 (d, J_{C-F}=246.8 Hz), 163.4 (d, J_{C-F}=243.2 Hz), 151.8, 144.1 (d, J_{C-F}=13.0 Hz), 137.4 (d, J_{C-F}=3.1 Hz), 130.5 (d, J_{C-F}=8.0 Hz), 122.7 (d, J_{C-F}=11.4 Hz), 115.9 (d, J_{C-F}=21.5 Hz). 112.1, 108.6 (d, J_{C-F}=26.3 Hz), 95.9 (d, J_{C-F}=26.5 Hz), 47.7. HRMS (ESI): Exact mass calculated for C₁₄H₁₂F₂N₃⁺ [M+H], 260.0999. found 260.1003. IC₅₀: 4.7 μM [3.7±0.58, n=3; 5.6±2.3, n=3]

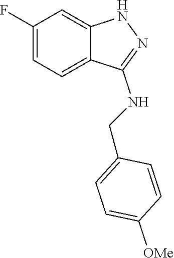 [SMILES: COc1ccc(CNc2n[nH]c3cc(F)ccc23)cc1]

5-fluoro-N-(3-methoxybenzyl)-1H-indazol-3-amine (9d). General Procedure B was used with the corresponding aldehyde to afford 9d (61 mg, 41%) as a pale yellow solid. Analytical data: ¹H NMR (500 MHz, Methanol-d₄) δ 7.67 (dd, J=8.8, 5.2 Hz, 1H), 7.40-7.26 (m, 2H), 6.92 (dd, J=9.8, 2.2 Hz, 1H), 6.88-6.82 (m, 2H), 6.72 (td, J=9.1, 2.2 Hz, 1H), 4.44 (s, 2H), 3.75 (s, 3H). ¹³C NMR (126 MHz, Methanol-d₄) 163.2 (d, J_{C-F}=242.6 Hz), 162.2, 158.9. 150.6, 142.7 (d, J_{C-F}=13.0 Hz). 131.9, 128.6, 121.4 (d, J_{C-F}=11.4 Hz), 113.4, 110.8, 107.2 (d, J_{C-F}=26.3 Hz), 94.45 (d, J_{C-F}=26.5 Hz), 54.3, 46.7. HRMS (ESI): Exact mass calculated for C₁₅H₁₅FN₃O⁺ [M+H], 272.1199. Found 272.1198. IC₅₀: 8.7 μM [7.4±1.2, n=3; 9.9±2.8, n=3]

### Procedure C: General Synthesis of Compounds 10a-e

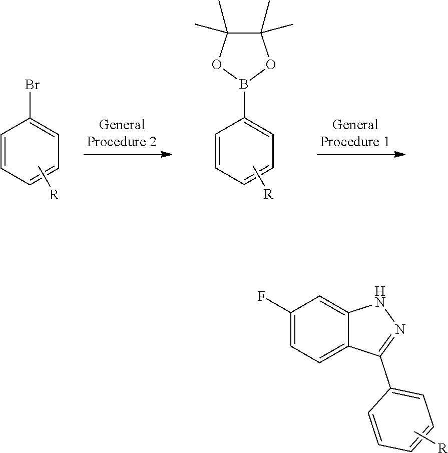

Compounds 10a-f were made through synthesis through a sequence with General Procedure 2 followed by General Procedure 1. unless otherwise noted. Some compounds can be washed with dichloromethane to remove further impurity.

### Tabulated Data of Compounds 10a-e

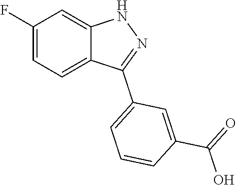 [SMILES: O=C(O)c1cccc(-c2n[nH]c3cc(F)ccc23)c1]

3-(6-fluoro-1H-indazol-3-yl)benzoic acid (10a). General Procedure 1 was used with S6 (1.00 g, 1 equiv.) and 3-methoxycarbonylphenylboronic acid (596 mg, 1.2 equiv.) to afford the precursor to 10a, which was dissolved in a 1:1 mixture of THF:Methanol (0.2 M each) and 8 equiv of NaOH (1.0 M) and stirred for 16 hours. After acidify, filtering, and washing with water, 10a was obtained in 53% yield (377 mg) over 2 steps as an ashy white solid. Analytical data: ¹H NMR (500 MHz, DMSO-d₆) δ 13.42 (s, 1H), 13.14 (s, 1H), 8.53 (t, J=1.8 Hz, 1H), 8.23 (dt, J=7.9, 1.5 H, 1H), 8.08 (dd, J=8.9, 5.1 Hz, 1H), 7.98 (dt, J=7.7, 1.5 Hz, 1H), 7.66 (t, J=7.7 Hz, 1H), 7.40 (dd, J_{C-F}=9.4, 2.3 Hz, 1H), 7.13 (td, J=9.1, 2,3 Hz, 1H). ¹³C NMR (126 MHz, DMSO-d₆) δ 167.2, 161.3 (d, J_{C-F}=242.2 Hz), 142.6, 141.9 (d, J_{C-F}=12.9 Hz), 133.5, 131.5, 130.9, 129.4, 128.6, 127.3, 122.3 (d, J_{C-F}=11.1 Hz), 117.15, 111.0 (d, J_{C-F}=26.1 Hz), 96.2 (d, J_{C-F}=25.8 Hz). HRMS (ESI): Exact mass calculated for C₁₄H₁₀FN₂O₂⁺ [M+H], 257.0726, found 257.0726. IC₅₀: 0.096 μM, [0.082±0.026, n=3; 0.11±0.010, n=3]

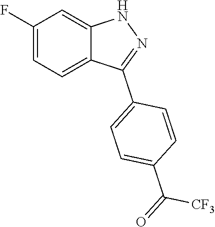 [SMILES: O=C(c1ccc(-c2n[nH]c3cc(F)ccc23)cc1)C(F)(F)F]

2,2,2-trifluoro-1-(4-(6-fluoro-1H-indazol-3-yl)phenyl)ethan-1-one (10b). General Procedure C was used to afford 10b (67 mg, 39%) as a yellow solid. Analytical data: ¹H NMR (500 MHz, Methanol-d₄) δ 8.07 (dd, J=9.0, 5.0 Hz, 1H), 8.04-7.99 (m, 2H), 7.79 (d, J=8.2 Hz, 2H), 7.27 (dd, J=9.2, 2.2 Hz, 1H), 7.07 (td, J=9.1, 2.2 Hz, 1H), ¹³C NMR (126 MHz, Methanol-d₄) δ 163.8 (d, J_{C-F}=243.9 Hz), 145.7, 143.6 (d, J_{C-F}=12.6 Hz), 136.0, 135.6, 129.9, 128.1, 124.5 (q, J_{C-F}=287.0 Hz), 123.8 (d, J_{C-F}=11.0 Hz), 118.9, 112.2 (d, J_{C-F}=26.4 Hz), 97.9 (q, J_{C-F}=31.1 Hz), 96.7 (d, J_{C-F}=26.5 Hz). HRMS (ESI): Exact mass calculated for C₁₅H₉F₄N₂O⁺ [M+H], 309.0651. Found 309.0645. IC₅₀: 0.60 μM [0.32±0.12, n=3; 0.88±0.071, n=3]

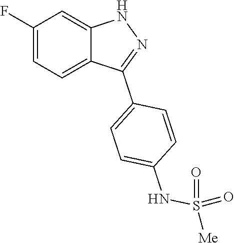 [SMILES: CS(=O)(=O)Nc1ccc(-c2n[nH]c3cc(F)ccc23)cc1]

N-(4-(6-fluoro-1H-indazol-3-yl)phenyl)methanesulfonamide (10c). General Procedure 3 was used on the corresponding boronic acid with MsCl, followed by General Procedure 1 with S6 to afford 10c (394 mg, 94%) as an off-white solid. Analytical data: ¹H NMR (500 MHz, DMSO-d₆) δ 13.24 (s, 1H), 9.90 (s, 1H), 8.08 (dd, J=8.9, 5.1 Hz, 1H), 7.96-7.92 (m, 2H), 7.35 (dd, J=9.1, 2.6 Hz, 3H), 7.07 (td, J=9.1, 2.3 Hz, 1H), 3.05 (s, 3H). ¹³C NMR (126 MHz, DMSO-₆) δ 161.2 (d, J=241.9 Hz), 143.1, 141.8 (d, J=12.9 Hz), 138.2, 128.7, 127.7, 122.6 (d, J=11.0 Hz), 119.8, 117.2, 110.5 (d, J=25.9 Hz), 95.9 (d, J=25.6 Hz). HRMS (ESI): Exact mass calculated for C₁₄H₁₂FN₃O₂S⁺ [M+H] 306.071.2. Found 306.0705, IC₅₀: 0.48 μM [0.33±0.26, n=3; 0.62±0.25, n=3]

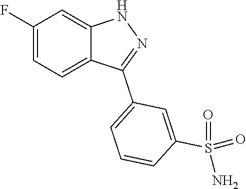 [SMILES: NS(=O)(=O)c1cccc(-c2n[nH]c3cc(F)ccc23)c1]

3-(6-fluoro-1H-indazol-3-yl)benzenesulfonanaide (10d). General Procedure C was used to afford 10d (108 mg, 54%) as an off-white solid. Analytical data: ¹H NMR (500 MHz, DMSO-d₆) δ 13.50 (bs, 1H), 8.42 (t, 1.8 Hz, 1H), 8.21 (dt, J=7.7, 1.3 Hz, 1H), 8.14 (dd, =9.0, 5.1 Hz, 1H), 7.85 (ddd, J=7.8, 1.9, 1.1 Hz, 1H), 7.72 (t, J=7.8 Hz, 1H), 7.48 (s, 2H), 7.42 (dd, J=9.4, 2.2 Hz, 1H), 7.16 (td, J=9.2, 2.3 Hz, 1H). ¹³C NMR (126 MHz, DMSO-₆) δ 161.3 (d, J_{C-F}=242.4 Hz), 144.8, 142.2, 141.9 (d, J_{C-F}=12.8 Hz), 133.9, 129.7, 124.8, 123.6, 122.4 (d, J_{C-F}=11.1 Hz), 117.1, 111.1 (d, J_{C-F}=26.1 Hz), 96.2 (d, J_{C-F}=25.7 Hz), HRMS (ESI): Exact mass calculated for C₁₃H₉FN₃O₂SNa⁺ [M+Na], 314.0376. Found 314.0374. IC₅₀: 3.1 μM [2.5±0.99, n=3; 3.6±0.34, n=3]

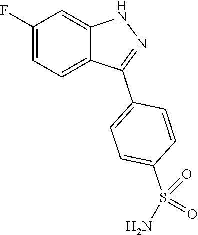 [SMILES: NS(=O)(=O)c1ccc(-c2n[nH]c3cc(F)ccc23)cc1]

4-(6-fluoro-1H-indazol-3-yl)benzenesulfonamide (10e). General Procedure C listed above was used to afford 10f (72 mg, 45%) as an off-white solid. Analytical data: ¹H NMR (500 MHz, DMSO-₆) δ 13.51 (s, 1H), 8.25-8.10 (m, 3H), 7.98-7.93 (m, 2H), 7.42 (d, J=9.2 Hz, 3H), 7.14 (td, J=9.1, 2.3 Hz, 1H). ¹³C NMR (126 MHz, DMSO-₆) δ 161.3 (d, J_{C-F}=242.5 Hz), 143.1, 142.2, 141.9 (d, J_{C-F}=12.9 Hz), 136.4, 127.0, 126.4, 122.6, 122.5, 117.3, 111.1 (d, J_{C-F}=26.1 Hz), 96.2 (d, J=25.9 Hz). HRMS (EST): Exact mass calculated for C₁₃H₁₁FN₃O₂S⁺ [M+H], 292.0556. Found 292.0555. IC₅₀: 0.061 μM [0.057±0.011, n=3; 0.065±0.011, n=3] MEK7 IC₅₀: 0.54 μM [0.52±0.67, n=3; 0.56±0.22, n=3]

### Procedure D: General Synthesis of Compounds 11-12

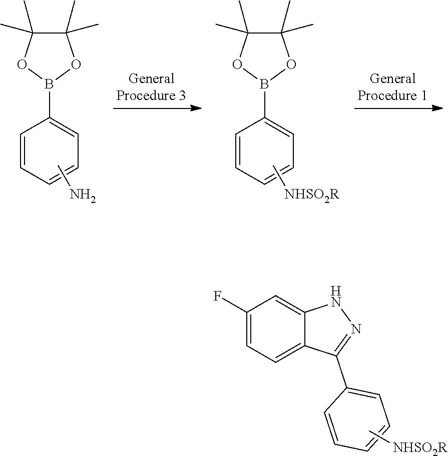

Compounds 11-12 were made through synthesis through the corresponding sulfonyl chloride and borylated aniline with General Procedure 3, followed by General Procedure 1, unless otherwise noted. Some compounds can he washed with dichloromethane to remove further impurity.

### Tabulated Data of Compounds 11a-d

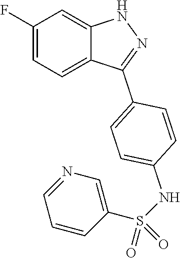 [SMILES: O=S(=O)(Nc1ccc(-c2n[nH]c3cc(F)ccc23)cc1)c1cccnc1]

N-(4-(6-fluoro-1H-indazol-3-yl)phenyl)pyridine-3-sulfonamide (11a). General Procedure D was used to afford 11a (128 mg, 55%) as a white solid. Analytical data: ¹H NMR (500 MHz, DMSO-d₆) δ 1124 (s, 1H), 10.66 (s, 1H), 8.93 (dd, J=2.3, 0.8 Hz, 1H), 8.79 (dd, J=4.8, 1.6 Hz, 1H), 8.16 (ddd, J=8.1, 2.4, 1.6 Hz, 1H), 8.03 (dd, J=9.0, 5.1 Hz, 1H), 7.88 (d, J=8.6 Hz, 2H), 7.62 (ddd, J=8.1, 4.8, 0.9 Hz, 1H), 7.33 (dd, J=9.5, 2.3 Hz, 1H), 7.26 (d, J=8.7 Hz, 2H), 7.04 (td, J=9.2, 2.3 Hz, 1H). ¹³C NMR (126 MHz, DMSO-d₆) δ 161.2 (d, J_{C-F}=242.0 Hz), 153.6, 147.1, 142.8, 141.8 (d, J_{C-F}=12.7 Hz), 136.8, 135.9, 134.7, 129.6, 127.7, 124.4, 122.6 (d, J_{C-F}=11.1 Hz), 120.8, 117.1, 110.5 (d, J_{C-F}=25.9 Hz), 95.9 (d, J_{C-F}=25.7 Hz). HRMS (ESI): Exact mass calculated for C₁₈H₁₄FN₄O₂S⁺ [M+H], 369.0821. Found 369.0825. IC₅₀: 0.75 μM [0.49±0.39, n=3; 1.0±0.23, n=3]

MEK7 IC₅₀: 2.7 μM [1.9±0.41, n=3; 3.5±1.5, n=3 μM]

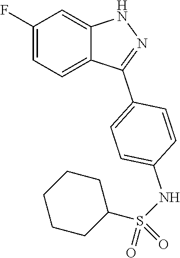 [SMILES: O=S(=O)(Nc1ccc(-c2n[nH]c3cc(F)ccc23)cc1)C1CCCCC1]

N-(4-(6-fluoro-1H-indazol-3-yl)phenyl)cyclohexanesulfonamide (11b). General Procedure D was used to afford 11b (172 mg, 79%) as an off-white solid. Analytical data: ¹H NMR (500 MHz, DMSO-d₆) δ 13.24 (s, 1H), 9.94 (s, 1H), 8.08 (dd, J=9.0, 5.1 Hz, 1H), 7.96-7.86 (m, 2H), 7.41-7.31 (m, 3H), 7.06 (td, J=9.2, 2.3 Hz, 1H), 3.04 (tt, J=11.9, 3.4 Hz, 1H), 2.11-2.00 (m, 2H), 1.76 (dt, J=12.9, 3.3 Hz, 2H), 1.58 (d, J=12.4 Hz, 1H), 1.44 (qd, J=12.5, 3.6 Hz, 2H), 1.28-1.16 (m, 2), 1.11 (qt, J=14.4, 3.8 Hz, 1H). ¹³C NMR (126 MHz, DMSO-d₆) δ 161.3 (d, J_{C-F}=242.1 Hz), 143.1, 141.8 (d, J_{C-F}=12.4 Hz), 138.5, 128.38, 127.7, 122.7 (d, J_{C-F}=11.0 Hz), 119.3, 117.2, 110.5 (d, J_{C-F}=25.8 Hz), 95.9 (d, J_{C-F}=25.8 Hz), 59.0, 26.0, 24.8, 24.4. HRMS (ESI): Mass calculated for C₁₉H₂₀ FN₃O₂SNa⁺ [M+Na], 396.1158. Found 396.1158. IC₅₀: 1.9 μM [1.8±2.1, n=3; 1.9±0.61, n=3]

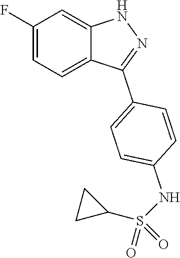 [SMILES: O=S(=O)(Nc1ccc(-c2n[nH]c3cc(F)ccc23)cc1)C1CC1]

N-(4-(6-fluoro-1H-indazol-3-yl)phenyl)cyclopropanesulfonamide (11c). General Procedure D was used to afford 11e (157 mg, 79%) as a pale yellow solid. Analytical data: ¹H NMR (500 MHz, DMSO-d₆) δ 13.23 (s, 1H), 9.88 (s, 1H), 8.08 (dd, J=9.0, 5.1 Hz, 1H), 7.96-7.91 (m, 2H), 7.40-7.37 (m, 2H), 7.35 (dd, J=9.5, 2.2 Hz, 1H), 7.07 (td, J=9.2, 2.3 Hz, 1H), 2.68 (tt, J=7.4, 5.2 Hz, 1H), 0.97 (tq, J=8.1, 2.7 Hz, 4H). ¹³H NMR (126 MHz, DMSO-d₆) δ 161.2 (d, J_{C-F}=241.8 Hz), 143.1, 141.8 (d, J_{C-F}=12.4 Hz), 138.2, 128.8, 127.6, 120.3, 117.2, 110.4 (d, J_{C-F}=26.0 Hz), 95.9 (d, J_{C-F}=25.7 Hz), 29.6, 5.0. HRMS (ESI): C₁₆H₁₅F₃O₂S⁺ [M+H], 322.0869. Found 322.0864, IC₅₀: 4.41 μM [4.3±2.2, n=3; 4.5±0.99, n=3]

### Tabulated Data of Compounds 12a-c

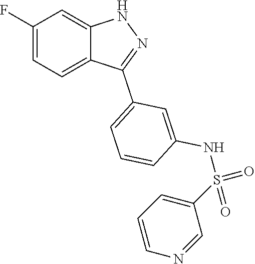 [SMILES: O=S(=O)(Nc1cccc(-c2n[nH]c3cc(F)ccc23)c1)c1cccnc1]

N-(3-(6-fluoro-1H-indazol-3-yl)phenyl)pyridine-3-sulfonamide (12a). General Procedure D was used to afford 12a (92 mg, 45%) as an off-white solid. Analytical data: ¹H NMR (500 MHz, DMSO-d₆) δ 13.33 (s, 1H), 10.65 (s, 1H), 8.93 (dd, J=2.4, 0.8 Hz, 1H), 8.78 (dd, J=4.8, 1.6 Hz, 1H), 8.16 (ddd, J=8.1, 2.4, 1.6 Hz, 1H), 7.91 (dd, J=9.0, 5.1 Hz, 1H), 7.73 (t, J=2.0 Hz, 1H), 7.68 (dt, J=7.8, 1.3 Hz, 1H), 7.61 (ddd, J=8.1, 4.8, 0.8 Hz, 1H), 7.41 (t, J=7.9 Hz, 1H), 7.37 (dd, J=9.5, 2.3 Hz, 1H), 7.18 (ddd, J=8.1, 2.3, 1.0 Hz, 1H), 7.11 (td, J=9.2, 2.3 Hz, 1H). ¹³C NMR (126 MHz, DMSO-d₆) δ 161.2 (d, J_{C-F}=242.3 Hz), 153.6, 147.1, 142.6, 141.8 (d, J_{C-F}=12.9 Hz), 137.6, 135.7, 134.7, 134.2, 130.0, 124.5, 122.9, 122.1 (d, J_{C-F}=11.1 Hz), 119.3, 118.7, 117.1, 110.8 (d, J_{C-F}=26.1 Hz), 96.1 (d, J_{C-F}=25.8 Hz). HRMS (ESI): Exact mass calculated for C₁₈H₁₄FN₄O₂S⁺ [M+H], 369.0821. Found 369.0822, IC₅₀: 1.3 μM [1.2±0.23, n=3; 1.3±0.39, n=3]

MEK7 IC₅₀: 1.3 μM [0.94±0.26, n=3; 1.6±0.61, n=3]

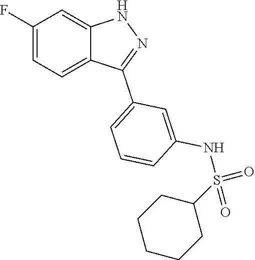 [SMILES: O=S(=O)(Nc1cccc(-c2n[nH]c3cc(F)ccc23)c1)C1CCCCC1]

N-(3-(6-fluoro-1H-indazol-3-yl)phenyl)cyclohexanesulfonamide (12b). General Procedure D was used to afford 12b (211 mg, 91%) as a yellow solid. Analytical data: ¹H NMR (500 MHz, DMSO-d₆) δ13.33 (s, 1H), 9.90 (s, 1H), 8.04 (dd, J=9.0, 5.1 Hz, 1H), 7.89 (t, J=2.0 Hz, 1H), 7.67 (dt, J=7.8. 1.2 Hz, 1H), 7.45 (t, J=7.9 Hz, 1H), 7.38 (dd, J=9.5, 2.3 Hz, 1H), 7.28 (ddd, J=8.1, 2.2, 1.0 Hz, 1H), 7.12 (td, J=9.1, 2.3 Hz, 1H), 3.03 (tt, J=11.9, 3.4 Hz, 1H), 2.11-2.00 (m, 2H), 1.75 (dt, J=13.0, 3.4 Hz, 2H), 1.56 (dt, J=12.2, 3.3 Hz, 1H), 1.44 (qd, J=12.4, 3.6 Hz, 2H), 1.20 (qt, J=12.5, 3.3 Hz, 2H), 1.11 (tt, J=12.7, 3.1 Hz, 1H). ¹³C NMR (126 MHz, DMSO-d₆) δ 161.3 (d, J_{C-F}=242.2 Hz), 143.0, 141.9 (d, J_{C-F}=12.8 Hz), 139.2, 134.2, 130.0, 122.3 (d. J_{C-F}=11.2 Hz), 121.7, 118.4, 117.3, 117.2, 110.8 (d, J_{C-F}=26.1 Hz). 96.1 (d, J_{C-F}=25.8 Hz), 73.5, 59.2, 26.0, 25.0, 24.8, 24.3. HRMS (ESI): Exact mass calculated for C₁₉H₂₁FN₃O₂S⁺ [M+H], 374.1338, Found 374.1329. IC₅₀: 14 μM [13±2.7, n=3; 15±4.4, n=3]

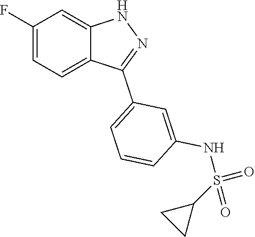 [SMILES: O=S(=O)(Nc1cccc(-c2n[nH]c3cc(F)ccc23)c1)C1CC1]

N-(3-(6-fluoro-1H-indazol-3-yl)phenyl)cyclopropanesulfonamide (12c). General Procedure D was used to afford 12c (203 mg, 67%) as a pale yellow solid. Analytical data: ¹H NMR (500 MHz, DMSO-d₆) δ 13.33 (s, 9.86 (s, 1H), 8.06 (dd, J=9.0, 5.1 Hz, 1H), 7.90 (t, J=1.9 Hz, 1H), 7.71 (dt, J=7.8, 1.3 Hz, 1H), 7.47 (t, J=7.9 Hz, 1H), 7.39 (dd, J=9.5, 2.2 Hz, 1H), 7.30 (ddd, J=8.1, 2.3, 1.0 Hz, 1H), 7.12 (td, J=9.1, 2.3 Hz, 1H), 2.67 (tt, J=7.7, 5.1 Hz, 1H), 1.03-0.88 (m, 4H). ¹³C NMR (126 MHz, DMSO-d₆) δ 161.7 (d, J_{C-F}=242.1 Hz), 143.5, 142.3 (d, J_{C-F}=12.9 Hz), 139.4, 134.6, 130.3, 122.8 (d, J_{C-F}=11.1 Hz), 118.8, 117.7, 111.2 (d, J_{C-F}=25.9 Hz), 96.6 (d, J_{C-F}=25.7 Hz), 30.1, 5.5. HRMS (ESI): Exact mass calculated for C₁₆H₁₄FN₃O₂S⁺ [M+Na], 354.0689. Found 354.0685, IC₅₀: 2.5 μM [2.1±0.51, n=3; 2.8±0.58, n=3]

MEK7 IC₅₀: 6.4 μM [5.6±2.2, n=3; 7.1±1.6, n=3]

### Procedure E: General Synthesis of Compounds 13-15

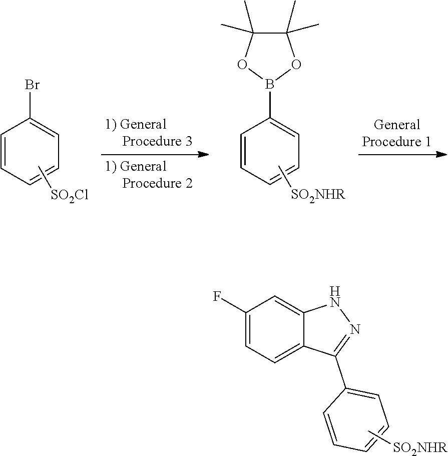

Compounds 13-15 were made through synthesis through the corresponding sulfonyl chloride and amine with General Procedure 3. followed by General Procedure 2 to produce the borylated product. The boronic ester was taken into General Procedure 1, to afford product, unless otherwise noted. Some compounds can be washed with dichloromethane to remove further impurity.

### Tabulated Data of Compounds 13a-c

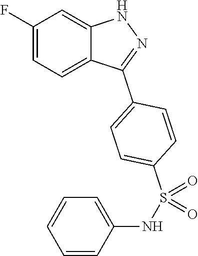 [SMILES: O=S(=O)(Nc1ccccc1)c1ccc(-c2n[nH]c3cc(F)ccc23)cc1]

4-(6-fluoro-1H-indazol-3-yl)-N-phenylbenzenesulfonamide (13a). General Procedure E was used to afford 13a (195 mg, 54%) as a pale yellow solid. Analytical data: ¹H NMR (500 MHz, DMSO-d₆) δ 13.54 (s, 1H), 10.35 (s, 1H), 8.18-8.10 (m, 3H), 7.91-7.85 (m, 2H), 7.40 (dd, J=9.4, 2.3 Hz, 1H), 7.28-7.21 (m, 2H), 7.16-7.08 (m, 3H), 7.03 (tt, J=7.3, 1.2 Hz, 1H). ¹³C NMR (126 MHz, DMSO-d₆) 161.2 (d, J_{C-F}=242.5 Hz), 142.0 (d, J_{C-F}=12.7 Hz), 141.7, 138.4, 137.7, 137.4, 129.2, 127.4, 127.1, 124.1, 122.6 (d, J_{C-F}=10.9 Hz), 120.1, 117.2, 111.2 (d, J_{C-F}=26.1 Hz), 96.2 (d, J_{C-F}=25.8 Hz). HRMS (ESI): Exact mass calculated for C₁₉H₁₄FN₃O₂SNa⁺ [M+Na], 390.0689. Found 390.0688. IC₅₀: 3.5 μM [2.4±1.0, n=3; 4.6±4.6, n=3]

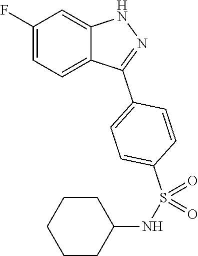 [SMILES: O=S(=O)(NC1CCCCC1)c1ccc(-c2n[nH]c3cc(F)ccc23)cc1]

N-cyclohexyl-4-(6-fluoro-1H-indazol-3-yl)benzenesulfonamide (13b). General Procedure E was used to afford 13b (121 mg, 81%) as an off-white solid. Analytical data: ¹H NMR (500 MHz, DMSO-d₆) δ 13.53 (s, 1H), 8.23-8.11 (m, 3H), 7.98-7.87 (m, 2H), 7.72 (d, J=7.3 Hz, 1H), 7.42 (dd, j=9.5, 2.3 Hz, 1H), 7.14 (t, J=9.4, 8.5 Hz, 1H), 3.03-2.93 (m, 1H), 1.65-1.54 (m, 4H), 1.44 (d, J=12.6 Hz, 1H), 1.19-1.08 (m, 4H), 1.07-1.00 (m, 1H). ¹³C NMR (126 Hz, DMSO-d₆) δ 161.8 (d, J_{C-F}=242.5 Hz), 142.5, 142.4 (d, J_{C-F}=12.8 Hz), 141.8, 137.1, 127.6, 127.5, 123.1 (d, J_{C-F}=11.0 Hz), 117.7. 111.6 (d, J_{C-F}=26.0 Hz), 96.7 (d, J_{C-F}=25.9 Hz). 52.6, 33.7, 25.3. 24.8. HRMS (ESI): Exact mass calculated for C₁₉H₂₀FN₃O₂SNa⁺ [M+Na], 396.1158. Found 396.1159. IC₅₀: 10 μM [9.1±3.5, n=3; 11±2.8, n=3]

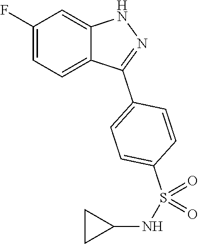 [SMILES: O=S(=O)(NC1CC1)c1ccc(-c2n[nH]c3cc(F)ccc23)cc1]

N-cyclopropyl-4-(6-fluoro-1H-indazol-3-yl)benzenesulfonamide (13c). General Procedure E was used to afford 13c (73 mg, 28%) as an off-white solid. Analytical data: ¹ H NMR (500 MHz, DMSO-d₆) δ 13.55 (bs, 1H), 8.26-8.20 (m, 2H), 8.19 (dd, J=9.0, 5.1 Hz, 1H), 8.04-7.97 (m, 1H), 7.96-7.91 (m, 2H), 7.42 (dd, J=9.4, 2.2 Hz, 1H), 7.14 (td, J=9.2, 2.3 Hz, 1H), 2.16 (tt, J=6.9, 3.6 Hz, 1H), 0.54-0.47 (m, 2H), 0.46-0.40 (m, 2H). ¹³C NMR (126 MHz, DMSO-d₆) δ 161.3 (d, J_{C-F}=242.5 Hz), 142.0, 141.9 (d, J_{C-F}=13.4 Hz), 139.2, 137.0, 127.5, 127.1, 122.6 (d, J_{C-F}=11.0 Hz), 117.3, 111.2 (d, J_{C-F}=25.9 Hz), 96.2 (d, J_{C-F}=25.9 Hz), 24.1, 5.2. HRMS (electrospray): Exact mass calculated for C₁₆H₁₄FN₃O₂SNa⁺ [M+Na], 354.0689. Found 354.0679. IC₅₀: 1.4 μM [1.3±0.41, n=3; 1.5±0.98, n=3]

MEK7 IC₅₀: 8.7 μM [8.1±2.3, n=3. 9.3±5.3, n=3 μM]

Tabulated Data of Compounds 14a-d

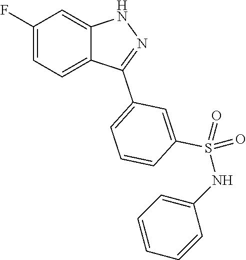 [SMILES: O=S(=O)(Nc1ccccc1)c1cccc(-c2n[nH]c3cc(F)ccc23)c1]

3-(6-fluoro-1H-indazol-3-yl)-N-phenylbenzenesulfonamide (14a). General Procedure E was used to afford 14a (151 mg, 54%) as a white solid. Analytical data: ¹H NMR (500 MHz, DMSO-d₆) δ 13.50 (s, 1H), 10.39 (s, 1H) 8.28 (t, J=1.8 Hz, 1H), 8.19 (dt, J=7.8, 1.4 Hz, 1H), 7.88 (dd, J=9.0, 5.1 Hz, 1H), 7.78 (ddd, J=7.9, 1.9, 1.1 Hz, 1H), 7.69 (t, J=7.8 Hz, 1H), 7.41 (dd, J=9.4, 2.2 Hz, 1H), 7.28-7.22 (m, 2H), 7.17-7.10 (m, 3H), 7.05 (tt, J=7.3, 1.2 Hz, 1H). ¹³C NMR (126 MHz, DMSO-d₆) δ 161.3 (d, J_{C-F}=242.6 Hz), 141.9 (d, J_{C-F}=12.7 Hz), 141.8, 140.2, 137.7, 134.1, 130.8, 130.1, 129.3, 125.9, 124.3, 124.3, 122.1 (d, J_{C-F}=11.1 Hz), 120.4, 117.0, 111.2 (d, J_{C-F}=26.1 Hz). 96.3 (d, J_{C-F}=25.7 Hz). HRMS (ESI): Exact mass calculated for C₁₉H₁₅FN₃O₂S⁺ [M+H], 368.0869. Found 368.0857. IC₅₀: 0.31 μM [0.30±0.058, n=3; 0.31±0.051 μM]

MEK7 IC₅₀: 0.84 μM [0.40±0.11, n3; 0.61±0.13, n=3: 1.5±0.30, n=3]

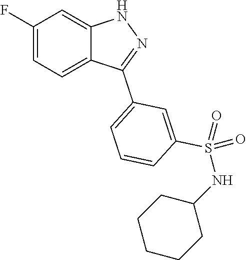 [SMILES: O=S(=O)(NC1CCCCC1)c1cccc(-c2n[nH]c3cc(F)ccc23)c1]

N-cyclohexyl-3-(6-fluoro-1H-indazol-3-yl)benzenesulfonamide (14b). General Procedure E was used to afford 14b (350 mg, 85%) as a pale yellow solid. Analytical data: ¹H NMR (500 MHz, DMSO-d₆) δ 13.51 (s, 1H), 8.40 (t, J=1.8 Hz, 1H), 8.23 (dt, J=7.8, 1.4 Hz, 1H), 8.13 (dd, J=9.0, 5.1 Hz, 1H), 7.87-7.78 (m, 2H), 7.73 (t, J=7.8 Hz, 1H), 7.42 (dd, J=9.4, 2.3 Hz, 7.16 (td, J=9.2. 2.3 Hz, 1H), 3.05-2.92 (m, 1H), 1.58 (h, J=5.1 Hz, 4H), 1.43 (d, J=12.4 Hz, 1H), 1.13 (td, J=9.9, 8.6, 5.5 Hz, 4H), 1.04 (t, J=10.9 Hz, 1H). ¹³C NMR (126 MHz, DMSO-d₆) δ 161.3 (d, J_{C-F}=242.5 Hz), 143.0, 142.0 (d, J_{C-F}=12.8 Hz), 134.1, 130.1, 130.0, 126.5, 124.0, 122.3 (d, J_{C-F}=1 Hz), 117.1, 111.1 (d, J_{C-F}=26.1 Hz), 96.3 (d, J_{C-F}=25.9 Hz), 52.2, 33.2, 24.8, 24.3. HRMS (ESI): Exact mass calculated for C₁₉H₂₁FN₃O₂S⁺ [M+H], 374.1338. Found 374.1342. IC₅₀: 0.86 μM [0.84±0.20, n3; 0.87±0.30, n=3]

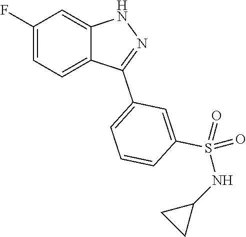 [SMILES: O=S(=O)(NC1CC1)c1cccc(-c2n[nH]c3cc(F)ccc23)c1]

N-cyclopropyl-3-(6-fluoro-1H-indazol-3-yl)benzenesulfonamide (14c). General Procedure E was used to afford 14c (158 mg. 49%) as a pale yellow solid. Analytical data: ¹H NMR (500 MHz, DMSO-d₆) δ 13.51 (s, 1H), 8.40 (s, 1H), 8.27 (d, J=7.9, 1.3 Hz, 1H), 8.12 (dd, J=9.0, 5.1 Hz, 1H), 8.06 (d, J=2.7 Hz, 1H), 7.87-7.82 (m, 1H), 7.77 (t, J=7.8 Hz, 1H), 7.43 (dd, J=9.5, 2.3 Hz, 1H), 7.16 (td, J=9.1, 2.2 Hz, 1H), 2.16 (tq, J=6.8, 3.3 Hz, 1H), 0.50 (dt, J=6.8, 3.3 Hz, 2H), 0.42 (q, J=3.7 Hz, 2H). ¹³C NMR (126 MHz, DMSO-d₆) δ 161.3 (d, J=242.5 Hz), 141.9, 141.5 (d, J_{C-F}=128.6 Hz), 134.1, 130.4, 130.0, 126.1, 124.5, 122.3 (d, J_{C-F}=11.0 Hz), 117.08, 111.2 (d, J_{C-F}=26.1 Hz), 96.3 (d, J_{C-F}=25.9 Hz), 24.2, 5.1. HRMS (ESI): Exact mass calculated for C₁₆H₁₅FN₃O₂S⁺ [M+H], 332.0869. Found 332.0863. IC₅₀: 0.38 μM [0.32±0.31, n=3; 0.44±0.065, n=3]

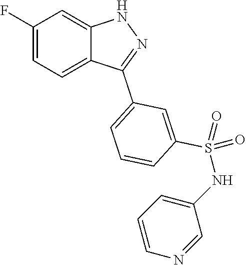 [SMILES: O=S(=O)(Nc1cccnc1)c1cccc(-c2n[nH]c3cc(F)ccc23)c1]

3-(6-fluoro-1-yl)-N-(pyridin-3-yl)benzenesulfonamide (14d). General Procedure E was used to afford 14d (196 mg, 38%) as a white solid. Analytical data: ¹H NMR (500 MHz, DMSO-d₆) δ 13.52 (s, 1H), 10.68 (bs, 1H), 8.35-8.30 (m, 2H), 8.27 (dd, J=4.7, 1.5 Hz, 1H), 8.24 (dt, J=7.8, 1.4 Hz, 1H), 7.96 (dd, J=9.0, 5.0 Hz, 1H), 7.78 (dt, J=8.0, 1.4 Hz, 1H), 7.71 (t, J=7.8 Hz, 1H), 7.56 (ddd, J=8.3, 2.7, 1.5 Hz, 1H), 7.42 (dd, J=9.4, 2.3 Hz, 1H), 7.31 (dd, J=8.3, 4.7 Hz, 1H) 7.14 (td, J=9.1, 2.3 Hz, 1H). ¹³C NMR (126 MHz, DMSO-d₆) δ 161.3 (d, J_{C-F}=242.6 Hz), 145.6, 142.0, 142.0, 141.9, 141.6, 139.8, 134.4, 134.3, 131.0, 130.3, 127.7, 125.9, 124.2 (d, J_{C-F}=14.0 Hz), 122.1 (d, J_{C-F}=11.1 Hz), 117.0, 111.2 (d, J_{C-F}=26.1 Hz), 96.3 (d, J_{C-F}=26.0 Hz), HRMS (ESI): Exact mass calculated for C₁₈H₁₃FN₄O₂S⁺ [M+H], 369.0821. Found 369.0821. IC₅₀: 0.45 μM [0.43±0.089, n=3; 0.46±0.10, n=3]

Tabulated Data of Compounds 15a-o

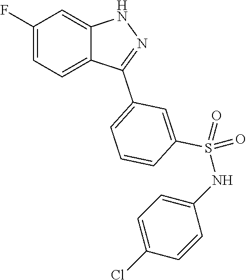 [SMILES: O=S(=O)(Nc1ccc(Cl)cc1)c1cccc(-c2n[nH]c3cc(F)ccc23)c1]

N-(4-chlorophenyl)-3-(6-fluoro-1H-indazol-3-yl)benzenesulfonamide (15a). General Procedure E was used to afford 15a (390 mg, 64%) as an off-white solid. Analytical data: ¹H NMR (500 MHz, DMSO-d₆) δ 13.52 (s, 1H), 10.55 (s, 1H), 8.29 (t, J=1.8 Hz, 1H), 8.22 (dt, J=7.8, 1.4 Hz, 1H), 7.91 (dd, J=9.0, 5.1 Hz, 1H), 7.77 (ddd, J=7.9, 1.9, 1.2 Hz, 1H), 7.70 (t, J=7.8 Hz, 1H), 7.42 (dd, J=9.4, 2.2 Hz, 1H), 7.35-7.29 (m, 2H), 7.19-7.09 (m, 3H). ¹³C NMR (126 MHz, DMSO-d₆) δ 161.3 (d, J_{C-F}=242.5 Hz), 141.9 (d, J_{C-F}=12.8 Hz), 141.7, 139.9, 136.7, 134.2, 130.9, 130.2, 129.3, 128.4, 125.9, 124.2, 122.0 (d, J_{C-F}=11.1 Hz), 117.0, 111.2 (d, J_{C-F}=26.1 Hz), 96.3 (d, J_{C-F}=25.8 Hz). HRMS (ESI): Calculated for C₁₉H₁₃ClFN₃O₂S⁺ 402.0474. Found 402.0481. IC₅₀: 1.4 μM [1.0±0.58, n=3, 1.8±0.79, n=3]

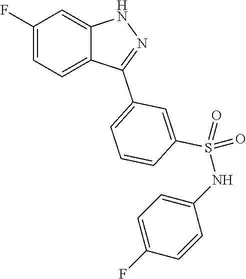 [SMILES: O=S(=O)(Nc1ccc(F)cc1)c1cccc(-c2n[nH]c3cc(F)ccc23)c1]

3-(6-fluoro-1H-indazol-3-yl)-N-(4-fluorophenyl)benzenesulfonamide (15b). General Procedure E was used to afford 15b (160 mg, 38%) as an off-white solid. Analytical data: ¹H NMR (500 MHz, DMSO-d₆) δ 13.51 (s, 1H), 10.35 (s, 1H), 8.26 (t, J=1.8 Hz, 1H), 8.21 (dt, J=7.5, 1.5 Hz, 1H), 7.92 (dd, J=9.0, 5.1 Hz, 1H), 7.75-7.66 (m, 2H), 7.41 (dd, J_{C-F}=9.5, 2.3 Hz, 1H), 7.16-7.07 (m, 5H). ¹³C NMR (126 MHz, DMSO-d₆) δ 161.2 (d, J=261.0 Hz), 159.3 (d, J_{C-F}=259.8 Hz), 141.9 (d, J_{C-F}=12.9 Hz), 141.7, 139.8, 134.1, 133.8, 133.8, 130.8, 130.1, 125.9, 124.3, 123.1 (d, J_{C-F}=8.4 Hz), 122.1 (d, J_{C-F}=11.0 Hz), 117.0, 116.0 (d, J_{C-F}=22.6 Hz), 111.12 (d, J_{C-F}=26.1 Hz), 96.31 (d, J_{C-F}=25.9 Hz). HRMS (ESI): Calculated for C₁₉H₁₄F₂N₃O₂S⁺ [M+H], 386.0775, Found 386.0768. IC₅₀: 0.88 μM [0.85±0.078, n=3; 0.91±0.11, n=3]

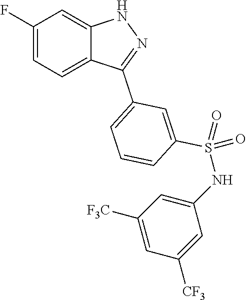 [SMILES: O=S(=O)(Nc1cc(C(F)(F)F)cc(C(F)(F)F)c1)c1cccc(-c2n[nH]c3cc(F)ccc23)c1]

N-(3,5-bis(trifluoromethyl)phenyl)-3-(6-fluoro-1H-indazol-3-yl)benzenesulfonamide (15c). General Procedure E was used to afford 15c (68 mg. 54%) as a white solid. Analytical data: ¹H NMR (500 MHz, DMSO-d₆) δ 13.55 (s, 1H), 11.32 (s, 1H), 8.36 (t, J=1.8 Hz, 1H), 8.27 (dt, J=7.8, 1.4 Hz, 1H), 7.95 (dd, J=9.0, 5.0 Hz, 1H), 7.84 (ddd, J=7.9, 1.9, 1.1 Hz, 1H), 7.80-7.70 (m, 4H), 7.42 (dd, J=9.4, 2.2 Hz, 1H), 7.12 (td, J=9.1, 2.3 Hz, 1H). ¹³C NMR (126 MHz, DMSO-d₆) δ 161.3 (d, J_{C-F}=242.8 Hz), 141.9 (d, J=12.9 Hz), 141.4, 140.1, 139.4, 134.5, 131.4. 131.3 (q, J_{C-F}=33.0 Hz), 130.51, 125.80, 124.1, 122.8 (q, J_{C-F}=272.9 Hz), 119.1 (d, J_{C-F}=3.2 Hz), 117.1 (tt, J_{C-F}=5.8, 3.1 Hz), 117.0, 111.2 (d, J_{C-F}=26.1 Hz), 96.4 (d, J_{C-F}=25.9 Hz). HRMS (ESI): Exact mass calculated for C₂₁H₁₃F₇N₃O₂S⁺ [M+H], 504.0616. Found 504.0619. IC₅₀: 3.3 μM [1.3±0.55, n=3; 5.2±1.3, n=3]

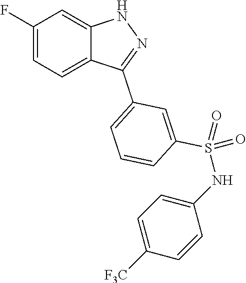 [SMILES: O=S(=O)(Nc1ccc(C(F)(F)F)cc1)c1cccc(-c2n[nH]c3cc(F)ccc23)c1]

3(6-fluoro-1H-indazol-3-(trifluoromethyl)phenyl)benzenesulfonamide (1.5d) General Procedure E was used to afford 15d (435 ma, 72%) as a yellow solid. Analytical data: ¹H NMR (500 MHz, DMSO-d₆) δ 13.53 (s, 1H), 10.99 (s, 1H), 8.37 (t, J=1.8 Hz, 1H), 8.24 (dt, J=7.9, 1.4 Hz, 1H), 7.96 (dd, J=9.0, 5.1 Hz, 1H), 7.85 (ddd, J=7.9, 2.0, 1.1 Hz, 1H), 7.72 (t, J=7.8 Hz, 1H), 7.63 (d, J=8.6 Hz, 2H), 7.42 (dd, J=9.4, 2.2 Hz, 1H), 7.35 (d, J=8.5 Hz, 2H), 7.13 (td, J=9.2, 2.3 Hz, 1H). ¹³C NMR (126 MHz, DMSO-d₆) δ 161.8 (d, J_{C-F}=242.61 Hz), 142.4 (d, J_{C-F}=12.9 Hz), 142.1, 140.5, 134.8, 131.6, 130.8, 127.2, 127.2 (q, J_{C-F}=3.8 Hz), 127.1, 126.3, 124.65 (d, J_{C-F}=271.4 Hz), 124.62, 122.5 (d, J_{C-F}=11.1 Hz), 119.5, 117.5, 111.6 (d, J_{C-F}=26.1 Hz), 96.8 (d, J_{C-F}=25.7 Hz). HRMS (ESI): Mass calculated for C₂₀H₁₃F₄N₃O₂SNa⁺ [M+Na], 458.0563. Found 458.0565. IC₅₀: 3.1 μM [2.3±0.34, n=3; 3.9±0.89, n=3]

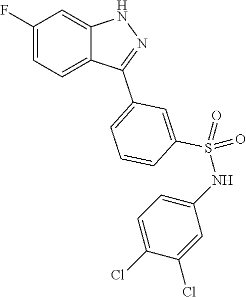 [SMILES: O=S(=O)(Nc1ccc(Cl)c(Cl)c1)c1cccc(-c2n[nH]c3cc(F)ccc23)c1]

N-(3,4-dichlorophenyl)-3-(6-fluoro-1H-indazol-3-yl)benzenesulfonamide (15e), General Procedure E was used to afford 15e (240 mg, 40%) as a white solid. Analytical data: ¹H NMR (500 MHz, DMSO-d₆) δ 13.54 (s, 1H), 10.82 (s, 1H), 8.32 (t, J=1.8 Hz, 1H), 8.25 (dt, J=7.7, 1.4 Hz, 1H), 7.94 (dd, J=9.0, 5.0 Hz, 1H), 7.81 (dt, J=7.9, 1.4 Hz, 1H), 7.73 (t, J=7.8 Hz, 1H), 7.53 (d, J=8.8 Hz, 1H), 7.42 (dd, J=9.4, 2.3 Hz, 1H), 7.35 (d, J=2.5 Hz, 1H), 7.18-7.11 (m, 2H). ¹³C NMR (126 MHz, DMSO-d₆) δ 161.3 (d, J_{C-F}=242.6 Hz), 141.9 (d, J_{C-F}=12.8 Hz), 141.6, 139.6, 137.9, 134.3, 131.5, 131.4, 131.2, 130.4, 126.3, 125.9, 124.2, 122.0 (d, J_{C-F}=11.1 Hz), 121.1, 119.9, 117.0, 111.2 (d, J_{C-F}=26.1 Hz), 96.4 (d, J_{C-F}=25.9 Hz). HRMS (ESI): Calculated for C₁₉H₁₂Cl₂FN₃O₂S⁺ [M+H], 436.0084, Found 436.0095. IC₅₀: 1.6 μM [1.4±0.99, n=3; 1.8±0.44, n=3]

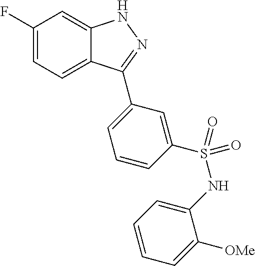 [SMILES: COc1ccccc1NS(=O)(=O)c1cccc(-c2n[nH]c3cc(F)ccc23)c1]

3-(6-fluoro-1H-indazol-3-yl)-N-(2-methoxyphenyl)benzenesulfonamide (15f). General Procedure E was used to afford 15f (540 mg, 70%) as a white solid. Analytical data: ¹H NMR (500 MHz, DMSO-d₆) δ 13.53 (s, 1H), 10.98 (s, 1H), 8,37 (t, J=1.8 Hz, 1H), 8.23 (dt, J=7.9, 1.4 Hz, 1H), 7.96 (dd, J=9.0, 5.1 Hz, 1H), 7.86 (ddt, J=9.3, 6.4, 1.9 Hz, 3H), 7.72 (t, J=7.9 Hz, 1H), 7.41 (dd, J=9.4, 2.3 Hz, 1H), 7.30-7.25 (m, 2H), 7.12 (td, J=9.1, 2.3 Hz, 1H), 2.45 (s, 3H). ¹³C NMR (126 MHz, DMSO-d₆) δ 196.5, 161.3 (d, J_{C-F}=242.5 Hz), 142.3, 141.9 (d, J_{C-F}=12.9 Hz), 141.6, 140.1, 134.3, 132.1, 131.1, 130.3, 129.9, 125.9, 124.2, 122.1 (d, J_{C-F}=11.1 Hz), 118.2, 117.0, 111.2 (d, J_{C-F}=26.1 Hz), 96.3 (d, J_{C-F}=25.8 Hz), 26.4. HRMS (ESI): Calculated for C₂₀H₁₆FN₃O₃S⁺ [M+H]; 398.0969. Found 398.0977. IC₅₀: 0.42 μM [0.28±0.13, n=3; 0.56±0.44, n=3]

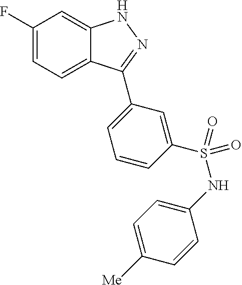 [SMILES: Cc1ccc(NS(=O)(=O)c2cccc(-c3n[nH]c4cc(F)ccc34)c2)cc1]

3-(6-fluoro-1H-indazol-3-yl)-N-(p-tolyl)benzenesulfonamide (15g). General Procedure E was used to afford 15g (367 mg, 70%) as a white solid. Analytical data: ¹ H NMR (500 MHz, DMSO-d₆) δ 8.27 (t, J=1.8 Hz, 1H), 8.19 (d, J=7.7 Hz, 1H), 7.90 (dd, J=9.0, 5.0 Hz, 1H), 7.77-7.72 (m, 1H), 7.68 (t, J=7.8 Hz, 1H), 7.41 (dd, J=9.4, 2.3 Hz, 1H), 7.12 (td, J=9.1. 2.3 Hz, 1H), 7.08-6.99 (m, 4H), 2.17 (s, 3H). ¹³C NMR (126 MHz, DMSO-d₆) δ 161.3 (d, J_{C-F}=242.6 Hz), 141.9 (d, J_{C-F}=12.8 Hz), 141.8, 140.2, 135.0, 134.0, 133.7, 130.7, 130.0, 129.7, 125.9, 124.3, 122.1 (d, J_{C-F}=11.1 Hz), 120.9, 117.0, 111.1 (d, J_{C-F}=26.1 Hz), 96.3 (d, J_{C-F}=25.8 Hz), 20.3. HRMS (ESI): Calculated for C₂₀H₁₇FN₃O₂S⁺ [M+H], 382.1026, found 382.1028. IC₅₀: 1.5 μM [1.0±0.10, n3; 1.9±0.82, n3]

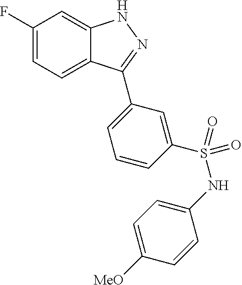 [SMILES: COc1ccc(NS(=O)(=O)c2cccc(-c3n[nH]c4cc(F)ccc34)c2)cc1]

3-(6-fluoro-1H-indazol-3-yl)-N-(4-methoxyphenyl)benzenesulfonamide (15h). General Procedure F was used to afford 15h (285 mg, 61%) as a white solid. Analytical data: ¹H NMR (500 MHz, DMSO-d₆) δ 13.50 (s, 1H), 10.03 (s, 1H), 8.24-8.22 (m, 1H), 8.19 (dt, J=7.1, 1.7 Hz, 1H), 7.87 (dd, J=9.0, 5.1 Hz, 1H), 7.74-7.63 (m, 2H), 7.41 (dd, J=9.4, 2.3 Hz, 1H), 7.11 (td, J=9.1, 2.3 Hz, 1H), 7.06-6.99 (m, 2H), 6.87-6.79 (m, 2H), 3.65 (s, 3H). ¹³C NMR (126 MHz, DMSO₆) δ 161.3 (d, J_{C-F}=242.6 Hz), 160.3, 156.7, 142.0, 141.9 (d, J_{C-F}=12.9 Hz), 140.1, 134.0, 130.0 (d, J_{C-F}=10.6 Hz), 130.0, 125.9, 124.4, 123.7, 122.1 (d, J_{C-F}=11.0 Hz), 117.0, 114.4, 111.0 (d, J_{C-F}=26.0 Hz), 96.3 (d, J_{C-F}=25.8 Hz), 55.1. HRMS (ESI): Exact mass calculated for C₂₀H₁₇FN₃O₃S⁺ [M+H], 398.0969. Found 398.0973. IC₅₀: 0.39 μM [0.24±0.14, n=3, 0.53±0.14, n=3]

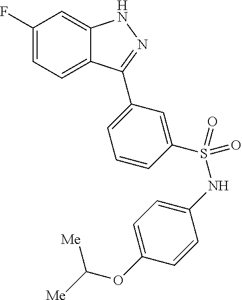 [SMILES: CC(C)Oc1ccc(NS(=O)(=O)c2cccc(-c3n[nH]c4cc(F)ccc34)c2)cc1]

3-(6-fluoro-1H-indazol-3-yl)-N-(4-isopropoxyphenyl)benzenesulfonamide (15i). General Procedure F was used to afford 15i (123 mg, 60%) as an ashy solid. Analytical data: ¹H NMR (500 MHz, DMSO-d₆) 13.42 (s, 1H), 9.93 (s, 1H), 8.24-8.06 (m, 2H), 7.79 (dd, J=9.0, 5.0 Hz, 1H), 7.67-7.58 (m, 2H), 7.34 (dd, J=9.4, 2.3 Hz, 1H), 7.05 (td, J=9.3, 2.3 Hz, 1H), 6.93 (d, J=7.8 Hz, 2H), 6.73 (d, J=8.8 Hz, 2H), 4.39 (hept, J=6.0 Hz, 1H), 1.09 (d, J=6.0 Hz, 6H). ¹³C NMR (126 MHz, DMSO₆) δ 161.8 (d, J_{C-F}=242.7 Hz), 155.4, 142.4 (d, J_{C-F}=13.1 Hz), 142.3, 134.4, 131.1, 130.4, 130.3, 126.4, 124.9, 124.2, 122.6 (d, J_{C-F}=11.0 Hz), 117.5, 116.6, 111.6 (d, J_{C-F}=26.1 Hz), 96.7 (d, J_{C-F}=25.8 Hz), 69.8, 39.5, 22.2. HRMS (ESI): Exact mass calculated for C₂₂H₂₁FN₃O₃S⁺ [M+H], 426.1287. Found 426.1288. IC₅₀: 6.1 μM [6.0±0.85, n=3; 6.1±5.0, n=3]

MEK7 IC₅₀: 2.9 μM [1.8±0.61, n=3; 2.2±0.82, n=3; 4.8±1.3, n=3]

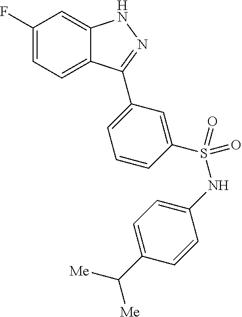 [SMILES: CC(C)c1ccc(NS(=O)(=O)c2cccc(-c3n[nH]c4cc(F)ccc34)c2)cc1]

3-(6-fluoro-1H-indazol-3-yl-(4-isopropylphenyl)benzenesulfonamide (15j). General Procedure E was used to afford 15j (141 mg, 58%) as an off-white solid. Analytical data: ¹H NMR (500 MHz, DMSO-d₆) δ 13.50 (s, 1H), 10.27 (s, 1H), 8.29 (t, J=1.8 Hz, 1H), 8.20 (dt, J=7.8, 1.4 Hz, 1H), 7.95 (dd, J=9.0, 5.0 Hz, 1H), 7.77 (ddd, J=7.9, 1.9, 1.1 Hz, 1H), 7.68 (t, J=7.8 Hz, 1H), 7.41 (dd, J=9.4, 2.2 Hz, 1H), 7.16-7.07 (m, 3H), 7.07-7.02 (m, 2H), 2.75 (hept, J=7.0 Hz, 1H), 1.08 (d, J=6.9 Hz, 6H). ¹³C NMR (126 MHz, DMSO-d₆) δ 161.3 (d, J_{C-F}=242.6 Hz), 144.5, 141.9 (d, J_{C-F}=12.8 Hz), 141.7, 140.3, 135.2, 134.1, 130.7, 130.1, 127.0, 125.9, 124.3, 111.1 (d, J_{C-F}=26.1 Hz), 96.3 (d, J_{C-F}=25.7 Hz), 32.7, 23.7. HRMS (ESI): Mass calculated for C₂₂H₂₀FN₃O₂SNa⁺ [M+Na], 432.1158. Found 432.1159, IC₅₀: 23 μM [10±7.1, n=3; 36±12, n=3]

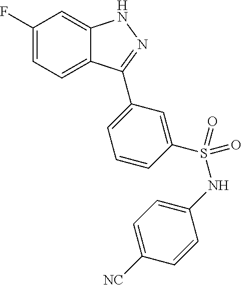 [SMILES: N#Cc1ccc(NS(=O)(=O)c2cccc(-c3n[nH]c4cc(F)ccc34)c2)cc1]

N-(4-cyanophenyl)-3-(6-fluoro-1H-indazol-3-yl)benzenesulfonamide (15k). General Procedure E used to afford 15k (224 mg, 46%) as an off-white solid. Analytical data: NMR (500 MHz, DMSO-₆) δ 13.53 (s, 1H), 11.15 (s, 1H), 8.36 (t, J=1.8 Hz, 1H), 8.24 (dt, J=7.9, 1.3 Hz, 1H), 7.97 (dd, J=9.0, 5.1 Hz, 1H), 7.86 (ddd, J=7.9, 2.0, 1.1 Hz, 1H), 7.76-7.67 (m, 3H), 7.42 (dd, j=9.5, 2.3 Hz, 1H), 7.29 (d, J=8.5 Hz, 2H), 7.15 (td, J=9.1, 2.3 Hz, 1H). ¹³C NMR (126 MHz, DMSO-d₆) δ 161.3 (d, J_{C-F}=243.0 Hz), 141.9 (d, J_{C-F}=12.7Hz), 141.6, 134.4, 133.7, 131.1, 130.3, 125.9, 124.1, 122.1 (d, J_{C-F}=10.9 Hz), 118.9, 117.0, 111.2 (d, J_{C-F}=26.0 Hz), 96.3 (d, J_{C-F}=25.9 Hz). HRMS (ESI): Exact mass calculated for C₂₀H₁₃FN₄O₂S⁺ [M+H], 393.0816. Found 393.0822. IC₅₀: 0.47 μM [0.21±0.023, n=3; 0.73±0.15, n=3]

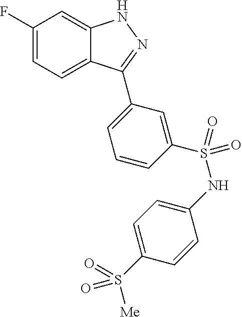 [SMILES: CS(=O)(=O)c1ccc(NS(=O)(=O)c2cccc(-c3n[nH]c4cc(F)ccc34)c2)cc1]

3-(6-fluoro-1H-indazol-3-yl))-N-(4-(methylsulfonyl)phenyl)benzenesulfonamide (15l). General Procedure E was used to afford 151 (336 mg, 55%) as a white solid. Analytical data: ¹H NMR (500 MHz, DMSO-d₆) δ 13.54 (s, 1H), 11.16 (s, 1H), 8.40 (t, J=1.8 Hz, 1H), 8.26 (dt, J=8.0, 1.4 Hz, 1H), 7.98 (dd, J=9.0, 5.0 Hz, 1H), 7.90 (ddd, J=7.9, 2.0, 1.1 Hz, 1H), 7.84-7.79 (m, 2H), 7.75 (t, J=7.8 Hz, 1H), 7.43 (dd, J=9.4, 2.2 Hz, 1H), 7.40-7.36 (m, 2H), 7.16 (td, J=9.1, 2.3 Hz, 1H), 3.10 (s, 3H). ¹³C NMR (126 MHz, DMSO-d₆) δ 161.3 (d, J_{C-F}=242.7 Hz), 142.5, 141.9 (d, J_{C-F}=12.9 Hz), 141.6, 140.0, 135.3, 134.4, 131.3, 130.4, 128.8, 125.9, 124.2, 122.6 (d, J_{C-F}=11.0 Hz), 119.0, 117.5, 111.7 (d, J_{C-F}=26.1 Hz), 96.8 (d, J_{C-F}=25.9 Hz), 44.1. HRMS (ESI): Exact mass calculated for C₂₀H₁₇FN₃O₄S₂⁺ [M+H], 446.0645. Found 446.0645 IC₅₀: 0.29 μM [0.29±0.058, n=3; 0.29±0.047, n=3]

MEK7 IC₅₀: 0.92 μM [0.59±0.19, n=3; 0.78±0.28, n=3; 1.4±0.34, n=3]

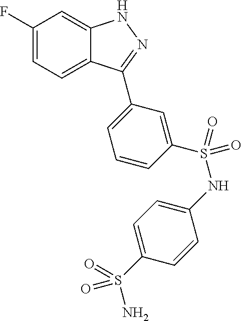 [SMILES: NS(=O)(=O)c1ccc(NS(=O)(=O)c2cccc(-c3n[nH]c4cc(F)ccc34)c2)cc1]

3-(6-fluoro-1H-indazol-3-yl)-N-(4-sulfanoylphenyl)benzenesulfonamide (15m). General Procedure E was used to afford 15m (287 mg, 27%) as an off-white solid. Analytical data: ¹ H NMR (500 MHz, DMSO-d₆) δ 13.54 (s, 1H), 10.98 (s, 1H), 8.38 (s, 1H), 8.23 (dd, J=7.8, 1.7 Hz, 1H), 7.98 (dd, J=9.0, 5.0 Hz, 1H), 7.86 (dt, J=8.0, 1.3 Hz, 1H), 7.76-7.67 (m, 3H), 7.42 (dd, J=9.5, 2.3 Hz, 1H), 7.31 (d, J=8.7 Hz, 2H), 7.21 (s, 2H), 7.16 (td, J=9.1, 2.3 Hz, 1H). ¹³C NMR (126 MHz, DMSO-d₆) δ 161.4 (d, J_{C-F}=242.6 Hz), 141.9 (d, J_{C-F}=12.9 Hz), 141.7, 140.9, 140.0, 139.1, 134.4, 131.2, 130.3, 127.3, 126.0, 124.3, 122.1 (d, J_{C-F}=11.1 Hz), 118.7, 117.0, 111.3 (d, J_{C-F}=26.1 Hz), 96.4 (d, J_{C-F}=25.8 Hz). HRMS (ESI): Mass calculated for C₁₉H₁₅FN₄O₄S₂Na⁺ [M+Na], 469.0417. Found 469.0416. IC₅₀: 0.64 μM [0.62±0.054, n=3; 0.65±0.071, n=3]

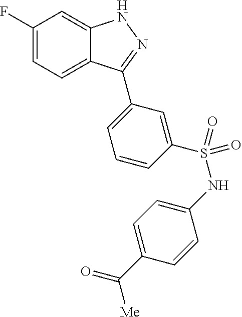 [SMILES: CC(=O)c1ccc(NS(=O)(=O)c2cccc(-c3n[nH]c4cc(F)ccc34)c2)cc1]

N-(4-acetylphenyl)-3-(6-fluoro-1H-indazol-3-yl)benzenesulfonamide (15n). General Procedure E was used to afford 15n (678 mg, 59%) as a white solid. Analytical data: ¹H NMR (500 MHz, DMSO-d₆) δ 13.53 (s, 1H), 10.98 (s, 1H), 8.37 (t, J=1.8 Hz, 1H), 8.23 (dt, J=7.9, 1.4 Hz, 1H), 7.96 (dd, J=9.0, 5.1 Hz, 1H), 7.86 (ddt, J=9.3, 6.4, 1.9 Hz, 3H), 7.72 (t, J=7.9 Hz, 1H), 7.41 (dd, J=9.4, 2.3 Hz, 1H), 7.30-7.25 (m, 2H), 7.12 (td, J=9.1, 2.3 Hz, 1H), 2.45 (s, 3H). ¹³C NMR (126 MHz, DMSO-d₆) 196.5, 161.3 (d, J_{C-F}=242.7 Hz), 142.2, 141.93 (d, J_{C-F}=12.9 Hz), 141.6, 140.0, 134.2, 132.2, 131.1, 130.3, 129.9, 125.9, 124.2, 122.1 (d, J_{C-F}=11.0 Hz), 118.2, 117.0. 111.2. (d, J_{C-F}=26.2 Hz), 96.3 (d, J_{C-F}=25.9 Hz), 26.4. HRMS (ESI): Exact mass calculated for C₂₁H₁₆FN₃O₃S⁺ [M+H], 410.0969. Found 410.0981. IC₅₀: 0.86 μM [0.81±0.095, n=3; 0.91±0.12, n=3]

MEK7 IC₅₀: 1.3 μM [0.74±0.25, n=3; 1.3±0.34, n=3; 1.8±0.37, n=3]

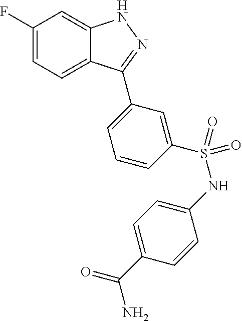 [SMILES: NC(=O)c1ccc(NS(=O)(=O)c2cccc(-c3n[nH]c4cc(F)ccc34)c2)cc1]

4-((3-(6-fluoro-1H-indazol-3-yl)phenyl)sulfonamido)benzamide (15o). General Procedure E was used to afford 15o (192 mg, 16%) as a white solid. Analytical data: ¹H NMR (500 MHz, DMSO-d₆) δ 13.51 (s, 1H), 10.75 (s, 1H), 8.30 (t, J=1.8 Hz, 1H), 8.21 (dt, J=7.8, 1.4 Hz, 1H), 7.88-7.79 (m, 3H), 7.78-7.74 (m, 2H), 7.71 (t, J=7.8 Hz, 1H), 7.41 (dd, J=9.4, 2.3 Hz, 1H), 7.24 (bs, 1H), 7.22-7.17 (m, 2H), 7.12 (td, J=9.2, 2.3 Hz, 1H). ¹³C NMR (126 MHz, DMSO-d₆) δ 167.1, 161.3 (d, J_{C-F}=242.5 Hz), 141.9 (d, J_{C-F}=12.8 Hz), 141.7, 140.4, 140.0, 134.2, 131.2, 130.2, 129.7, 128.9, 125.9, 124.2, 122.0 (d, J_{C-F}=11.1 Hz), 118.7, 117.0, 111.2 (d, J_{C-F}=26.1 Hz), 96.3 (d, J_{C-F}=25.7 Hz). HRMS (ESI): Exact mass calculated for C₂₀H₁₅FN₄O₃S⁺ [M+H], 411.0922. Found 411.0928.1 IC₅₀: 0.083 μM [0.075±0.011, n=3; 0.088±0.011, n=3]

MEK7 IC₅₀: 0.096 μM [0.041±0.014, n=3; 0.15±0.064, n=3]

Tabulated Data for Compounds S1-5

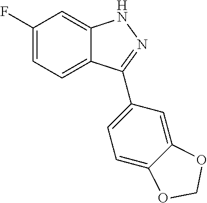 [SMILES: Fc1ccc2c(-c3ccc4c(c3)OCO4)n[nH]c2c1]

3-(benzo[d][1,3]dioxol-5-yl)-6-fluoro-1H-indazole (S1). Using General Procedure 1 with the corresponding boronic acid, S1 (218 mg, 77%) was obtained as a brown solid. Spectra and potency match literature,³ Analytical data: ¹H NMR (500 MHz, DMSO-d₆) δ 13.19 (s, 1H), 8.03 (dd, J=8.9, 5.1 Hz, 1H), 7.56-7.41 (m, 2H), 7.34 (dd, J=9.5, 2.3 Hz, 1H), 7.12-6.99 (m, 2H), 6.09 (s, 2H). ¹³C NMR (126 MHz, DMSO-₆) δ 161.2 (d, J_{C-F}=241.8 Hz), 147.8, 147.1, 143.4, 141.8 (d, J_{C-F}=12.8 Hz), 127.3, 122.6 (d, J_{C-F}=11.1 Hz), 120.6, 117.1, 110.4 (d, J_{C-F}=25.9 Hz), 108.7, 107.0, 101.2, 95.8 (d, J_{C-F}=25.7 Hz). IC₅₀: 0.026±0.013 μM, n=3

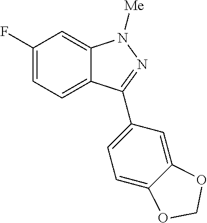 [SMILES: Cn1nc(-c2ccc3c(c2)OCO3)c2ccc(F)cc21]

3-(benzo[d][1,3]dioxol-5-yl)-6-fluoro-1-methyl-1H-indazole (S2). To a flame-dried vial was added S1 (70 mg, 0.27 mmol). it was dissolved in acetone (0.68 mL), and the solution reduced to 0° C. followed by addition of KOH (23 mg, 0.41 mmol). After 15 minutes of stirring, MeI (17 μL, 0.27 mmol) was added dropwise. Reaction was monitored by UPLC. At 1 hour, more MeI was added dropwise (34 μL, 0.54 mmol). The reaction was washed with saturated aqueous ammonium chloride solution and concentrated. Residue was purified by flash chromatography (0-30% EtOAc/Hex, SiO₂) to yield S2 (44 mg, 60%) as an off-white solid. Analytical data: ¹H NMR (500 MHz, DMSO-d₆) δ 8.03 (dd, J=9.0, 5.2 Hz, 1H), 7.56 (dd, J=9.8, 2.2 Hz, 1H), 7.48-7.39 (m, 2H), 7.10-7.02 (m, 2H), 6.09 (s, 2H), 4.04 (s, 3H). ¹³C NMR (126 MHz, DMSO-₆) δ 161.3 (d, J_{C-F}=242.2 Hz), 147.8, 147.2, 142.3, 141.6 (d, J_{C-F}=12.9 Hz), 126.8, 122.8 (d, J_{C-F}=11.1 Hz), 120.6, 117.5, 110.5 (d, J_{C-F}=26.1 Hz), 108.7, 106.9, 101.2, 95.7 (d, J_{C-F}=26.5 Hz). HRMS (ESI): Exact mass calculated for C₁₅H₁₂FN₂O₂⁺[M+H], 271.0883. Found 271.0876. IC₅₀:>80 μM

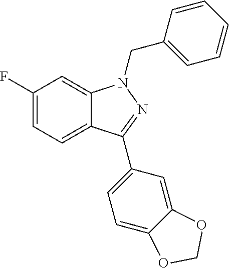 [SMILES: Fc1ccc2c(-c3ccc4c(c3)OCO4)nn(Cc3ccccc3)c2c1]

3-(benzo[d][1,3]dioxol-5-yl)-1-benzyl-6-fluoro-1H-indazole (S3). To a flame-dried vial was added S1 (70 mg, 0.27 mmol). It was dissolved in acetone (0.68 mL). The solution was reduced to 0° C. and KOH (23 mg, 0.41 mmol) was added. After 15 minutes of stirring, benzyl bromide (32 μL, 0.27 mmol) was added dropwise. Reaction was monitored by UPLC. Reaction was concentrated and reconstituted in ethyl acetate. The organic layer was washed with water and brine, dried over sodium sulfate, concentrated in vacuo, and purified by flash chromatography (0-30% EtOAc/Hex, SiO₂) to yield S3 (73 mg, 77%) as a white solid. Analytical data: ¹H NMR (500 MHz, DMSO-d₆) δ 8.05 (dd, J=8.9, 5.1 Hz, 1H), 7.67 (dd, J=9.8, 2.3 Hz, 1H), 7.49-7.41 (m, 2H), 7.35-7.28 (m, 4H), 7.26 (tt, J=7.2, 2.2 Hz, 1H), 7.11-7.03 (m, 2H), 6.09 (s, 2H), 5.65 (s, 2H). ¹³C NMR (126 MHz, DMSO-d₆) δ 161.4 (d, J_{C-F}=242.6 Hz), 114, 147.8, 147.3, 143.1, 141.3 (d, J_{C-F}=12.9 Hz), 137.1, 128.5, 127.6, 127.5, 126.7, 123.0 (d, J_{C-F}=11.1 Hz), 120.8, 117.8, 110.7 (d, J_{C-F}=26.0 Hz), 108.7, 107.0, 101.22, 95.9 (d, J_{C-F}=26.6 Hz), 51.8. HRMS (ESA): Exact mass calculated for C₂₁H₁₆FN₂O₂⁺ [M+H], 347.1196. Found 347.1190. IC₅₀:>80 μM

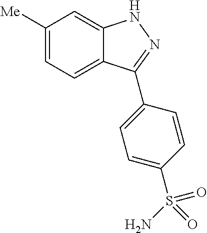 [SMILES: Cc1ccc2c(-c3ccc(S(N)(=O)=O)cc3)n[nH]c2c1]

4-(6-methyl-1H-indazol-3-yl)benzenesulfonamide (S4). The procedure used to make precursor S6 was used to make the corresponding indazole starting material for S4. General Procedure C was then used to afford S4 (66 mg, 17%) as a pink solid. Analytical data: ¹H NMR (500 MHz, DMSO-d₆) δ 13.28 (s, 1H), 8.21-8.15 (m, 2H), 8.02 (d, J==8.4 Hz, 1H), 7.96-7.92 (m, 2H), 7.45-7.37 (m, 3H), 7.09 (dd, J=8.5, 1.4 Hz, 1H), 2.47 (s, 3H), ¹³C NMR (126 MHz, DMSO) δ 142.7, 142.3, 141.5, 137.0, 136.1, 127.6, 126.7, 126.4, 126.3, 123.7, 120.2, 118.4, 110.0, 21.3. HRMS (ESI): Mass calculated for C₁₄H₁₃N₃O₂SNa⁺ [M+Na], 310.0626. Found 310.0624. IC₅₀: 2.7 μM [1.6±0.17, n=3; 3.7±0.95, n=3]

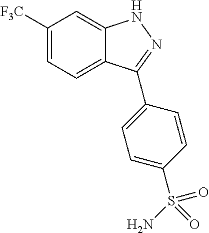 [SMILES: NS(=O)(=O)c1ccc(-c2n[nH]c3cc(C(F)(F)F)ccc23)cc1]

4-(6-(trifluoromethyl)-1H-indazol-3-yl)benzenesulfonanamide (S5). The procedure used to make precursor S6 was used to make the corresponding indazole starting material for S5, General Procedure C was then used to afford S5 (124 mg, 23%) as a pale orange solid. Analytical data: ¹H NMR (500 MHz, DMSO-d₆) δ 13.92 (bs, 1H), 8.38 (d, J=8.6 Hz, 1H), 8.25-8.20 (m, 2H), 8.02 (s, 1H), 8.00-7.96 (m, 2H), 7.53 (dd, J=8.7, 1.6 Hz, 1H), 7.44 (s, 2H). ¹³C NMR (126 MHz, DMSO-d₆) δ 143.4, 142.4, 140.5, 136.0, 127.1, 126.9, 126.8 (d, J_{C-F}=31.5 Hz), 126.4, 124.5 (d, J_{C-F}=272.2 Hz), 122.3, 121.9, 117.44 (q, J_{C-F}=3.0 Hz), 109.07-108.34 (m). HRMS (ESI): Mass calculated for C₁₄H₁₀F₃N₃O₂SNa⁺ [M+Na], 364.0344. Found 364.0343. IC₅₀: 3.7 μM [2.2±0.54, n=3; 5.2±1.3, n=3]

### REFERENCES

1. Mishra, R. K.; Deibler, K. K.; Clutter, M. R.; Vagadia, P. P.; O'Connor, M.; Schiltz, Cr. E.; Bergan, R.; Scheidt, K. A.; Modeling MEK4 Kinase Inhibitors through Perturbed Electrostatic Potential Charges. J. Chem. Inf. Model. 2019, 59, 4460-4466.
2. Lefebvre, V.; Cailly, T.; Fabis, F.; Rault, S.; Two-Step Synthesis of Substituted 3-Aminoindazoles from 2-Bromobenzonitriles. J. Org. Chem. 2010, 75, 2730-2732.
3. Deibler, K. K.; Schiltz, G. E.; Clutter, M. R.; .Mishra, R. K.; Vagadia, P. P.; O'Connor, M.; Donny George, M.; Gordon, R.; Fowler, G; Bergan, R.; Scheidt, K. A.; Synthesis and Biological Evaluation of 3-Arylindazoles as Selective MEK4 Inhibitors. ChemMedChem 2019, 14, 615-620.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A compound of Formula I, or a salt or hydrate thereof: 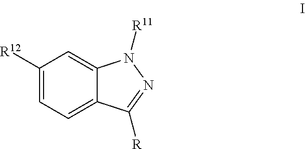
wherein
R is selected from the group consisting of —NR¹R², 1,3-benzodioxolyl, and aryl, wherein the aryl is optionally substituted with amino, carboxyl, —S(O)₂—NR₃R₄, —NR⁵—S(O)₂R⁶, or —NR⁷—C(O)R⁸;
R² is alkyl substituted with 1,3-benzodioxolyl or aryl, wherein the aryl is substituted with one or more hydroxyl, halo, or alkoxy;
R³ is selected from the group consisting of hydrogen, cycloalkyl, pyridyl, and aryl, wherein the aryl is optionally substituted with one or more substituents selected from the group consisting of halo, alkyl, haloalkyl, alkoxy, cyano, —S(O)₂-alkyl, —S(O)₂—NR⁹R¹⁰, —C(O)-alkyl, and —C(O)NR⁹R¹⁰;
R¹, R⁴, R⁵, R⁷, R⁹, and R¹⁰ are independently hydrogen or alkyl;
R⁶ is cycloalkyl;
R⁸ is selected from the group consisting of alkyl and alkenyl, wherein the alkenyl is optionally substituted with halo or amino;
R¹¹ is hydrogen, alkyl, or alkylaryl; and
R¹² is halo, alkyl, or haloalkyl.

2. The compound of claim 1, wherein the compound has a Formula I(a):

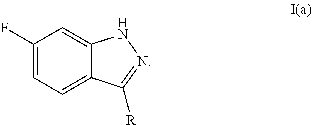

3. The compound of claim wherein R is phenyl optionally substituted with amino, carboxyl, or —NHC(O)R⁸; and
R⁸ is alkyl, vinyl, or propenyl, wherein the propenyl is substituted with halo or amino.

4. The compound of claim 3, wherein the compound is

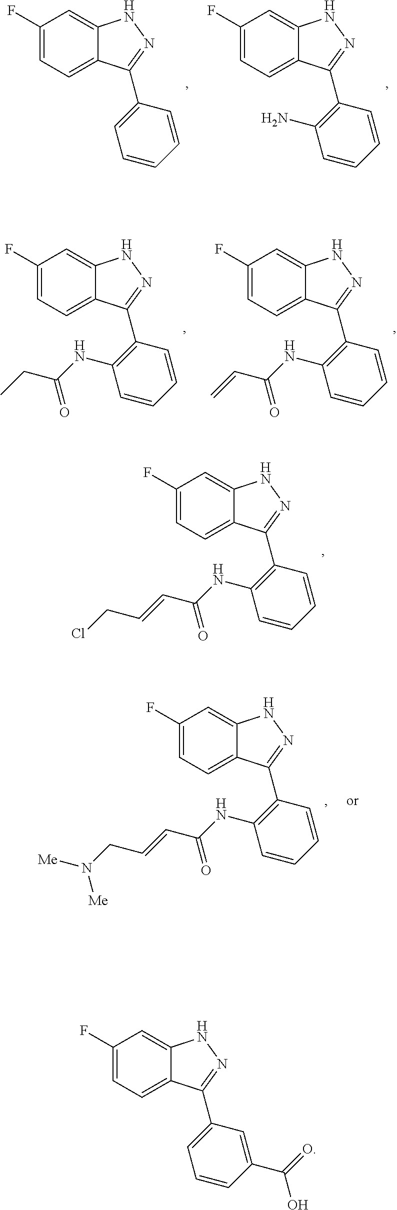

5. The compound of claim 2, wherein R is —NHR²; and
R² is methyl substituted with 1,3-benzodioxolyl or phenyl, wherein the phenyl is substituted with one or more hydroxyl, halo, or alkoxy.

6. The compound of claim 5, wherein the compound is

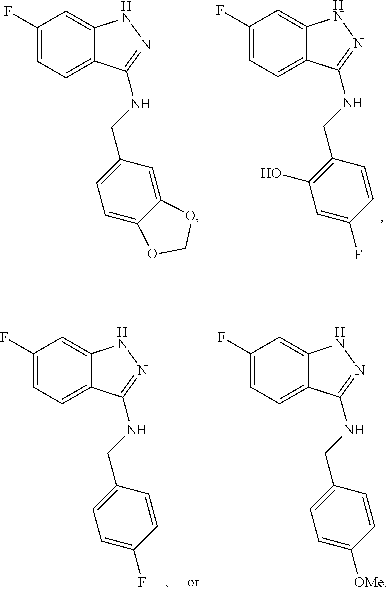 [SMILES: Fc1ccc2c(NCc3ccc4c(c3)OCO4)n[nH]c2c1 ; Oc1cc(F)ccc1CNc1n[nH]c2cc(F)ccc12 ; Fc1ccc(CNc2n[nH]c3cc(F)ccc23)cc1 ; COc1ccc(CNc2n[nH]c3cc(F)ccc23)cc1]

7. The compound of claim 2, wherein R is phenyl substituted with —NHS(O)₂R⁶; and
R⁶ is C₃-C₅ cycloalkyl.

8. The compound of claim 7, wherein the compound is

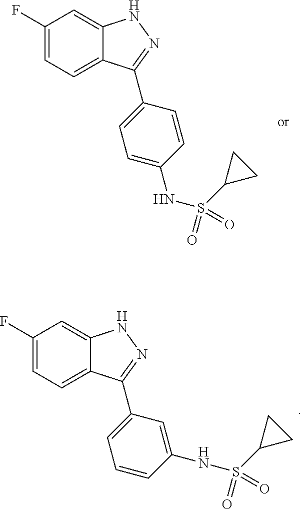 [SMILES: O=S(=O)(Nc1ccc(-c2n[nH]c3cc(F)ccc23)cc1)C1CC1 ; O=S(=O)(Nc1cccc(-c2n[nH]c3cc(F)ccc23)c1)C1CC1]

9. The compound of claim 2, wherein R is phenyl substituted with —S(O)₂—NHR³; and
R³ is selected from the group consisting of cycloalkyl, pyridyl, and phenyl, wherein the phenyl is optionally substituted with one or more substituents selected from the group consisting of halo, trifluoromethyl alkoxy, alkyl, cyano, —S(O)₂Me, —S(O)₂NH₂, acetyl, and —C(O)NH₂.

10. The compound of claim 9, wherein the compound is

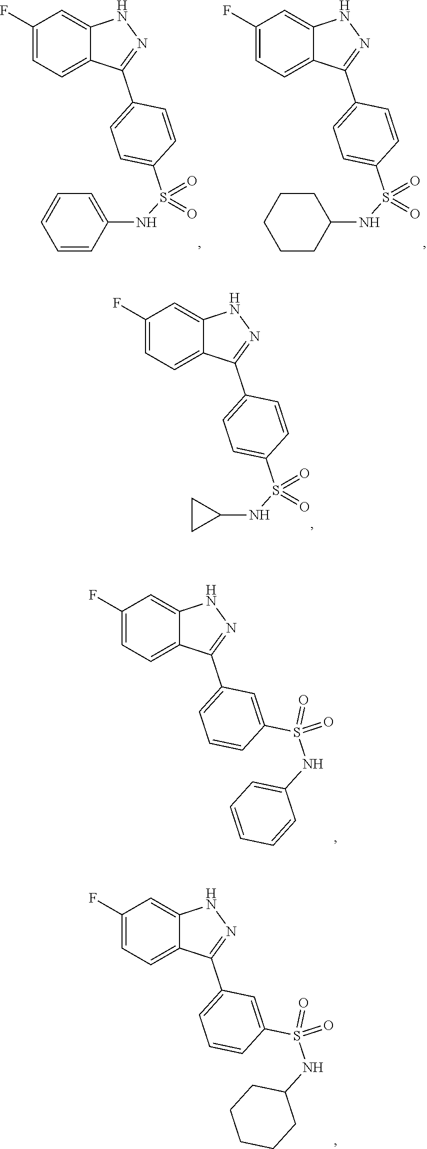 [SMILES: O=S(=O)(Nc1ccccc1)c1ccc(-c2n[nH]c3cc(F)ccc23)cc1 ; O=S(=O)(NC1CCCCC1)c1ccc(-c2n[nH]c3cc(F)ccc23)cc1 ; O=S(=O)(NC1CC1)c1ccc(-c2n[nH]c3cc(F)ccc23)cc1 ; O=S(=O)(Nc1ccccc1)c1cccc(-c2n[nH]c3cc(F)ccc23)c1 ; O=S(=O)(NC1CCCCC1)c1cccc(-c2n[nH]c3cc(F)ccc23)c1]

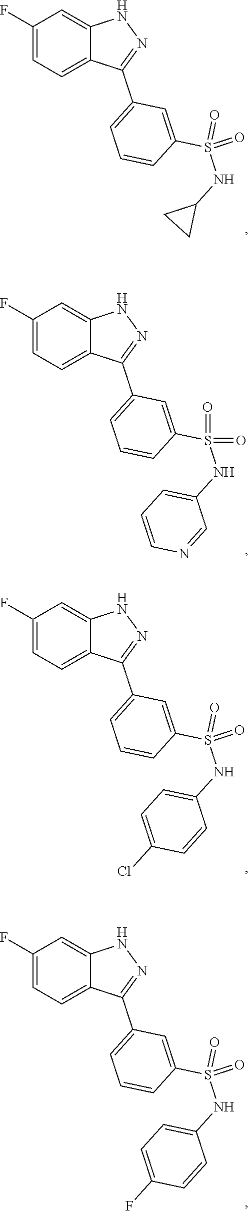 [SMILES: O=S(=O)(NC1CC1)c1cccc(-c2n[nH]c3cc(F)ccc23)c1 ; O=S(=O)(Nc1cccnc1)c1cccc(-c2n[nH]c3cc(F)ccc23)c1 ; O=S(=O)(Nc1ccc(Cl)cc1)c1cccc(-c2n[nH]c3cc(F)ccc23)c1 ; O=S(=O)(Nc1ccc(F)cc1)c1cccc(-c2n[nH]c3cc(F)ccc23)c1]

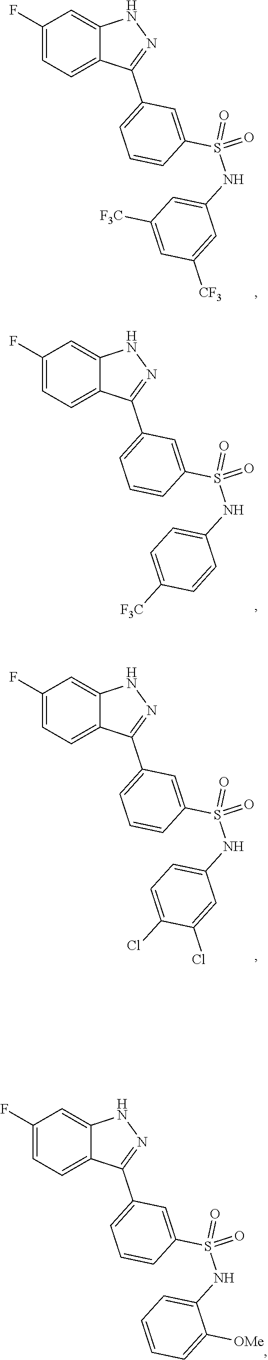 [SMILES: O=S(=O)(Nc1cc(C(F)(F)F)cc(C(F)(F)F)c1)c1cccc(-c2n[nH]c3cc(F)ccc23)c1 ; O=S(=O)(Nc1ccc(C(F)(F)F)cc1)c1cccc(-c2n[nH]c3cc(F)ccc23)c1 ; O=S(=O)(Nc1ccc(Cl)c(Cl)c1)c1cccc(-c2n[nH]c3cc(F)ccc23)c1 ; COc1ccccc1NS(=O)(=O)c1cccc(-c2n[nH]c3cc(F)ccc23)c1]

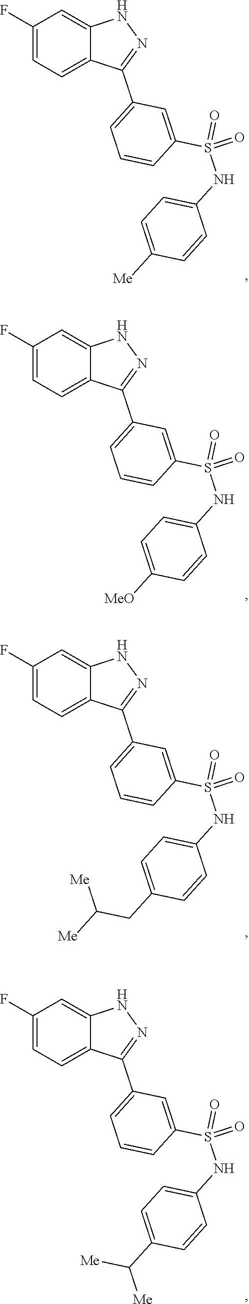 [SMILES: Cc1ccc(NS(=O)(=O)c2cccc(-c3n[nH]c4cc(F)ccc34)c2)cc1 ; COc1ccc(NS(=O)(=O)c2cccc(-c3n[nH]c4cc(F)ccc34)c2)cc1 ; CC(C)Cc1ccc(NS(=O)(=O)c2cccc(-c3n[nH]c4cc(F)ccc34)c2)cc1 ; CC(C)c1ccc(NS(=O)(=O)c2cccc(-c3n[nH]c4cc(F)ccc34)c2)cc1]

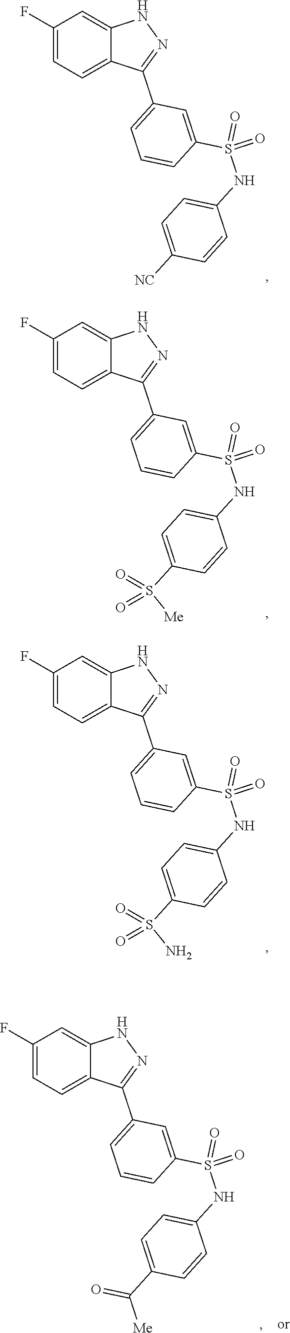 [SMILES: N#Cc1ccc(NS(=O)(=O)c2cccc(-c3n[nH]c4cc(F)ccc34)c2)cc1 ; CS(=O)(=O)c1ccc(NS(=O)(=O)c2cccc(-c3n[nH]c4cc(F)ccc34)c2)cc1 ; NS(=O)(=O)c1ccc(NS(=O)(=O)c2cccc(-c3n[nH]c4cc(F)ccc34)c2)cc1 ; CC(=O)c1ccc(NS(=O)(=O)c2cccc(-c3n[nH]c4cc(F)ccc34)c2)cc1]

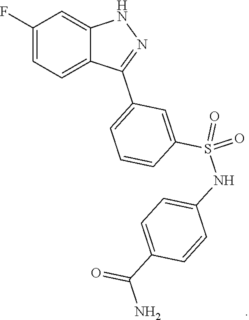 [SMILES: NC(=O)c1ccc(NS(=O)(=O)c2cccc(-c3n[nH]c4cc(F)ccc34)c2)cc1]

11. The compound of claim 1, wherein the compound is

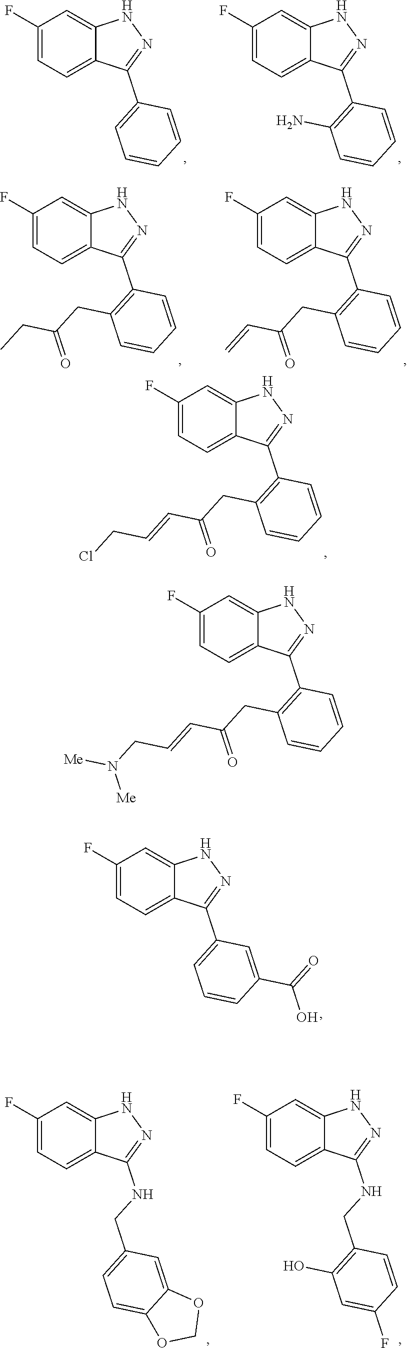

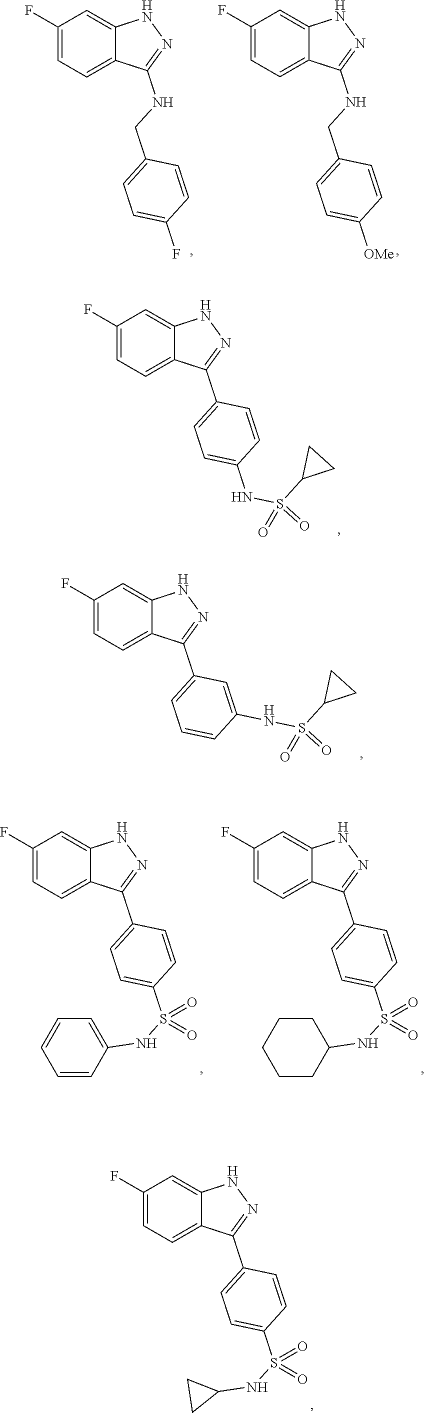

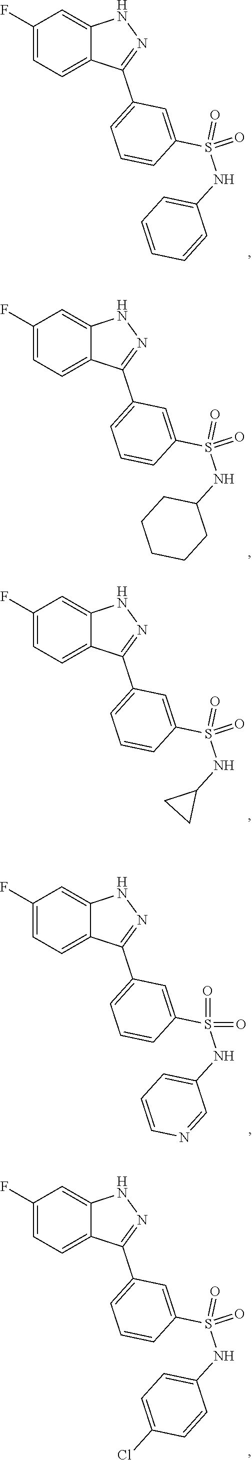 [SMILES: O=S(=O)(Nc1ccccc1)c1cccc(-c2n[nH]c3cc(F)ccc23)c1 ; O=S(=O)(NC1CCCCC1)c1cccc(-c2n[nH]c3cc(F)ccc23)c1 ; O=S(=O)(NC1CC1)c1cccc(-c2n[nH]c3cc(F)ccc23)c1 ; O=S(=O)(Nc1cccnc1)c1cccc(-c2n[nH]c3cc(F)ccc23)c1 ; O=S(=O)(Nc1ccc(Cl)cc1)c1cccc(-c2n[nH]c3cc(F)ccc23)c1]

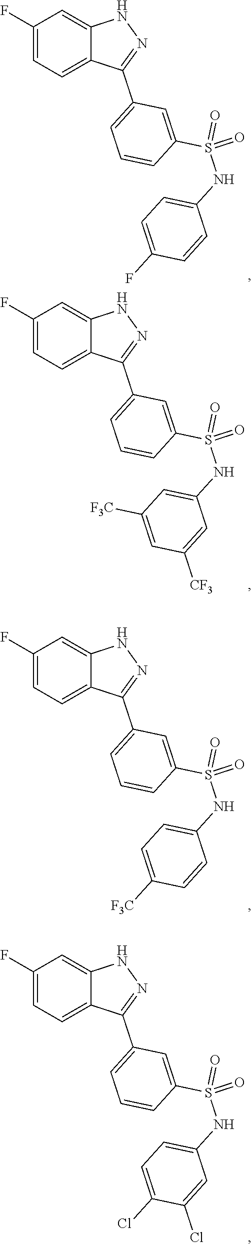

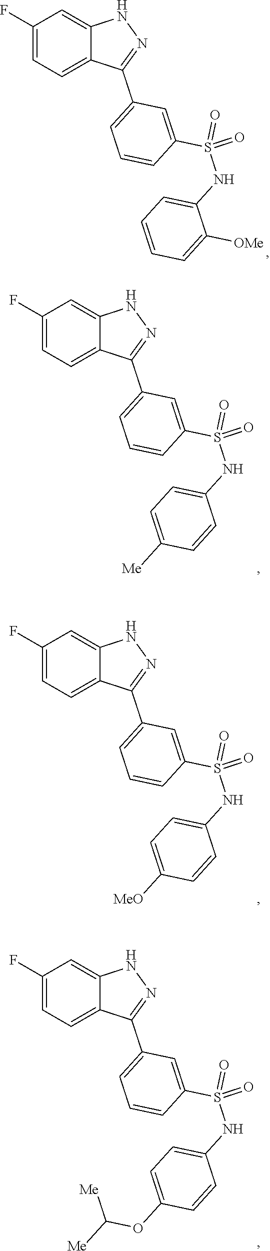 [SMILES: COc1ccccc1NS(=O)(=O)c1cccc(-c2n[nH]c3cc(F)ccc23)c1 ; Cc1ccc(NS(=O)(=O)c2cccc(-c3n[nH]c4cc(F)ccc34)c2)cc1 ; COc1ccc(NS(=O)(=O)c2cccc(-c3n[nH]c4cc(F)ccc34)c2)cc1 ; CC(C)Oc1ccc(NS(=O)(=O)c2cccc(-c3n[nH]c4cc(F)ccc34)c2)cc1]

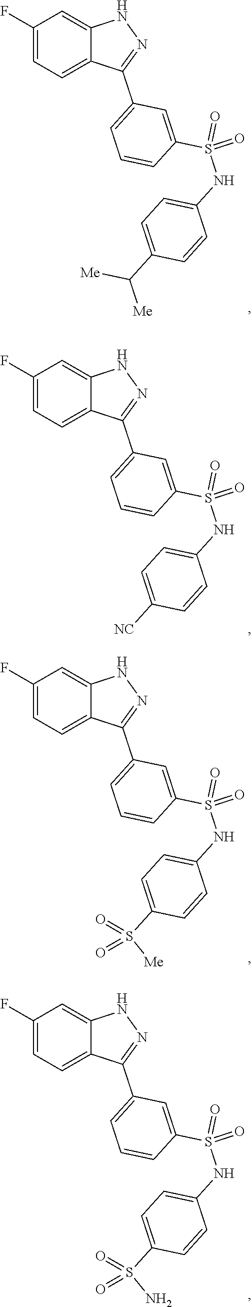

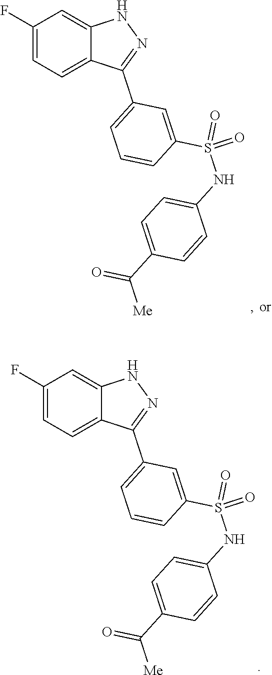 [SMILES: CC(=O)c1ccc(NS(=O)(=O)c2cccc(-c3n[nH]c4cc(F)ccc34)c2)cc1 ; CC(=O)c1ccc(NS(=O)(=O)c2cccc(-c3n[nH]c4cc(F)ccc34)c2)cc1]

12. The compound of claim 1, wherein the compound is

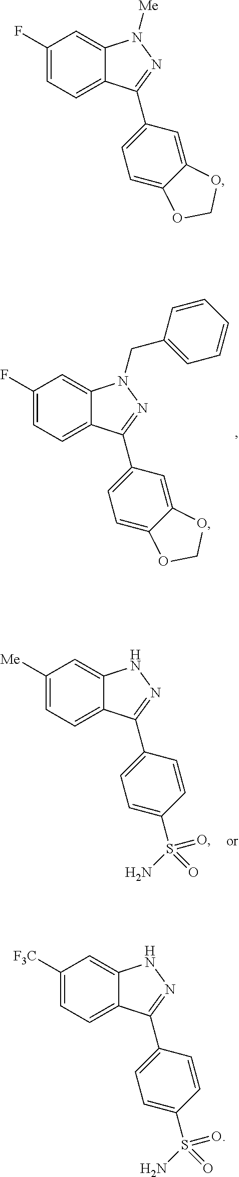 [SMILES: Cn1nc(-c2ccc3c(c2)OCO3)c2ccc(F)cc21 ; Fc1ccc2c(-c3ccc4c(c3)OCO4)nn(Cc3ccccc3)c2c1 ; Cc1ccc2c(-c3ccc(S(N)(=O)=O)cc3)n[nH]c2c1 ; NS(=O)(=O)c1ccc(-c2n[nH]c3cc(C(F)(F)F)ccc23)cc1]

13. The compound of claim 1, wherein the compound is

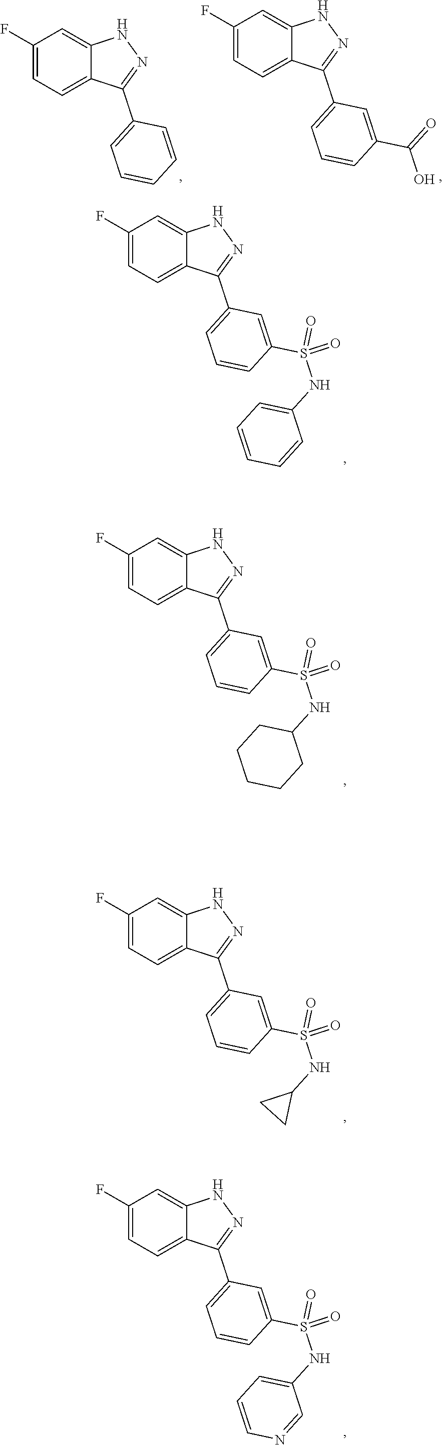

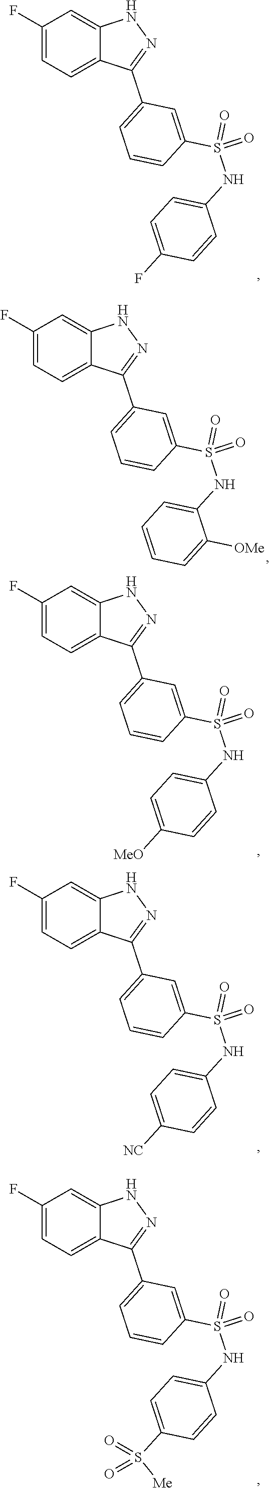

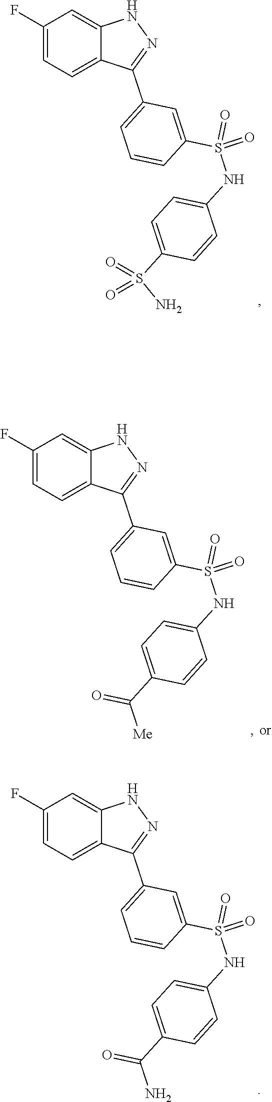 [SMILES: NS(=O)(=O)c1ccc(NS(=O)(=O)c2cccc(-c3n[nH]c4cc(F)ccc34)c2)cc1 ; CC(=O)c1ccc(NS(=O)(=O)c2cccc(-c3n[nH]c4cc(F)ccc34)c2)cc1 ; NC(=O)c1ccc(NS(=O)(=O)c2cccc(-c3n[nH]c4cc(F)ccc34)c2)cc1]

14. The compound of claim 1, wherein the compound is

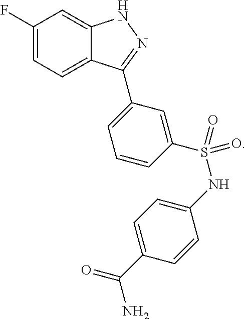 [SMILES: NC(=O)c1ccc(NS(=O)(=O)c2cccc(-c3n[nH]c4cc(F)ccc34)c2)cc1]

15. A pharmaceutical composition comprising the compound of claim 1, or a salt or hydrate thereof, and a pharmaceutical carrier, excipient, or diluent.

16. A method for treating a disease or disorder associated with mitogen-activated protein kinase 4 (MEK4) activity in a subject in need thereof, the method comprising administering to the subject the compound of claim 1, or a salt or hydrate thereof, or the pharmaceutical composition comprising the compound, or the sale or hydrate thereof, and a pharmaceutical carrier, excipient, or diluent.

17. The method of claim 16, wherein the disease or disorder is a cell proliferative disease or disorder.

18. The method of claim 17, wherein the cell proliferative disease or disorder is cancer.

19. The method of claim 18, wherein the cancer is selected from the group consisting of prostate cancer, breast cancer, pancreatic cancer, colon cancer, and combinations thereof.

20. The method of claim 19, wherein the cancer is pancreatic cancer.
